(19) United States
(12) Patent Application Publication
(10) Pub. No.: US 2006/0067886 A1
(43) Pub. Date: Mar. 30, 2006

(54) Gastrin receptor-avid peptide conjugates

(76) Inventors: Timothy J. Hoffman, Columbia, MO (US); Wynn A. Volkert, Columbia, MO (US); Gary Sieckman, Ashland, MO (US); Charles J. Smith, Columbia, MO (US); Hariprasad Gali, Columbia, MO (US)

Correspondence Address: KOHN & ASSOCIATES PLLC, 30500 NORTHWESTERN HWY, STE 410, FARMINGTON HILLS, MI, 48334, US

(21) Appl. No.: 11/267,001

(22) Filed: Nov. 4, 2005

Related U.S. Application Data
(63) Continuation of application No. 09/847,134, filed on May 2, 2001.
Application No. 09/847,134 is a continuation-in-part of application No. 09/537,423, filed on Mar. 29, 2000, now abandoned.
Application No. 09/537,423 is a division of application No. 09/064,499, filed on Apr. 22, 1998, now Pat. No. 6,200,546.
(60) Provisional application No. 60/044,049, filed on Apr. 22, 1997.

(51) Int. Cl. A61K 51/00; C07K 14/595

(52) U.S. Cl. 424/1.69; 530/309

(57) ABSTRACT

A compound for use as a therapeutic or diagnostic radiopharmaceutical includes a group capable of complexing a medically useful metal attached to a moiety which is capable of binding to a gastrin releasing peptide receptor. A method for treating a subject having a neoplastic disease includes administering to the subject an effective amount of a radiopharmaceutical having a metal chelated with a chelating group attached to a moiety capable of binding to a gastrin releasing peptide receptor expressed on tumor cells with subsequent internalization inside of the cell. A method of forming a therapeutic or diagnostic compound includes reacting a metal synthon with a chelating group covalently linked with a moiety capable of binding a gastrin releasing peptide receptor.

### CROSS-REFERENCE TO RELATED APPLICATIONS

This patent application is a continuation of U.S. patent application Ser. No. 09/847,134, filed May 2, 2001, which is a continuation-in-part of U.S. patent application Ser. No. 09/537,423, filed Mar. 29, 2000, which is a divisional of U.S. patent application Ser. No. 09/064,499, filed Apr. 22, 1998, which claims the benefit of priority to U.S. Provisional Application Ser. No. 60/044,049, filed on Apr. 22, 1997, all of which are incorporated herein by reference.

### GRANT REFERENCE

The research carried out in connection with this invention was supported in part by a grant from the Department of Energy (DOE), grant number DE-FG02-89ER60875, a grant from the U.S. Department of Veterans Affairs Medical Research Division and the Department of Radiology MU-C2-02691. The Government has certain rights in the invention.

### TECHNICAL FIELD

This invention relates to radionuclide-labeled compounds useful as radiopharmaceuticals. More particularly, the present invention relates to conjugates of bombesin (BBN) analogues and a metal complexing group which, when complexed to a radionuclide, are useful therapeutic and imaging agents for cancer cells that express gastrin releasing peptide (GRP) receptors.

### BACKGROUND OF THE INVENTION

Detection and treatment of cancers using radiopharmaceuticals that selectively target cancers in human patients has been employed for several decades. Unfortunately, only a limited number of site-directed radiopharmaceuticals that exhibit highly specific in vivo localization in or near cancer cells are currently in routine use, as being approved by the United States Food and Drug Administration (FDA). There is a great deal of interest in developing new radioactive drugs due to the emergence of more sophisticated biomolecular carriers that have high affinity and high specificity for in vivo targeting of tumors. Several types of agents are being developed and have been investigated including monoclonal antibodies (MAbs), antibody fragments (F_{AB}'s and (F_{AB})₂'s), receptor-avid peptides [Bushbaum, 1995; Fischman et al., 1993; Schubiger et al. 1996].

The potential utility of using radiolabeled receptor-avid peptides for producing radiopharmaceuticals is best exemplified by ¹¹¹In-DTPA-conjugated octreotide (an FDA approved diagnostic imaging agent, Octreoscan®, marketed in the United States. by Mallinckrodt Medical, Inc.) [Lowbertz et al. 1994]. This radiopharmaceutical is an ¹¹¹In-DTPA conjugate of Octreotide, a small peptide analogue of the human hormone somatostatin. This drug specifically binds to somatostatin receptors that are over-expressed on neuroendocrine cancers (e.g., carcinoid Ca, neuroblastoma, etc.) as well as others [Krenning et al., 1994]. Since indium-111 (¹¹¹In) is not the ideal radionuclide for scintigraphic imaging, other somatostatin analogues and other receptor-avid biomolecules that are labeled with ^{99m}Tc (the optimal radionuclide for diagnostic imaging) are being studied and developed [Eckelman, 1995; Vallabhajosula et al., 1996].

Bombesin (BBN) is a 14 amino acid peptide that is an analogue of human gastrin releasing peptide (GRP) that binds to GRP receptors with high specificity and has an affinity similar to GRP [Davis et al., 1992]. GRP receptors have been shown to be over-expressed or uniquely expressed on several types of cancer cells. Binding of GRP receptor agonists (also autocrine factors) increases the rate of cell division of these cancer cells. For this reason, a great deal of work has been, and is being pursued to develop BBN or GRP analogues that are antagonists [Davis et al., 1992; Hoffken, 1994; Moody et al., 1996; Coy et al., 1988; Cai et al., 1994]. These antagonists are designed to competitively inhibit endogenous GRP binding to GRP receptors and reduce the rate of cancer cell proliferation [Hoffken, 1994]. Treatment of cancers using these antagonists with these non-radioactive peptides requires chronic injection regimens (e.g., daily, using large quantities of the drug).

In designing an effective receptor-avid radiopharmaceutical for use as a diagnostic or therapeutic agent for cancer, it is important that the drug have appropriate in vivo targeting and pharmacokinetic properties [Fritzberg et al., 1992; Eckelman et al., 1993]. For example, it is essential that the radiolabeled receptor-avid peptide have high specific uptake by the cancer cells (e.g., via GRP receptors). In addition, it is necessary that once the radionuclide localizes at a tumor site, it must remain there for an extended time to deliver a highly localized radiation dose to the tumor. In order to achieve sufficiently high specific uptake of radiolabeled BBN analogues in tumors, the binding affinity of promising derivatives must be high (i.e., K_{d}≅1-5 nmolar or less) with prolonged retention of radioactivity [Eckelman et al., 1995; Eckelman, et al., 1993]. Work with ¹²⁵I-BBN derivatives has shown, however, that for cancer cells that bind the ¹²⁵I-BBN derivatives (whether they be agonists or antagonists), the radioactivity is either washed off or expelled from the cells (in vitro) at a rapid rate [Hoffman et al., 1997]. Thus, these types of derivatives have a low probability of remaining “trapped” at the tumor site (in vivo) sufficiently long to be effective therapeutic or diagnostic agents.

Developing radiolabeled peptides that are cleared efficiently from normal tissues is also an important and especially critical factor for therapeutic agents. When labeling biomolecules (e.g., MAb, F_{AB}'s or peptides) with metallic radionuclides (via a chelate conjugation), a large percentage of the metallic radionuclide (in some chemical form) usually becomes “trapped” in either the kidney or liver parenchyma (i.e., is not excreted into the urine or bile) [Duncan et al., 1997; Mattes, 1995]. For the smaller radiolabeled biomolecules (i.e., peptides or F_{AB}'s), the major route of clearance of activity is through the kidneys which in turn retain high levels of the radioactive metal (i.e., normally >10-15% of the injected dose) [Duncan et al., 1997]. This presents a major problem that must be overcome in the development of therapeutic agents that incorporate metallic radionuclides, otherwise the radiation dose to the kidneys would be excessive. For example, ¹¹¹In-octreotide, the FDA approved diagnostic agent, exhibits high uptake and retention in kidneys of patients [Eckelman et al., 1995]. Even though the radiation dose to the kidneys is higher than desirable, it is tolerable in that it is a diagnostic radiopharmaceutical (it does not emit alpha- or beta-particles), and the renal dose does not produce observable radiation induced damage to the organ.

It has been found that conjugating non-metallated metal chelates to BBN derivatives can form GRP agonists which exhibit binding affinities to GRP receptors that are either similar to or approximately an order of magnitude lower than the parent BBN derivative. [Li et al., 1996a] Our recent results show that it is now possible to add radiometal chelates to BBN analogues, to form conjugates which are agonists, and retain GRP receptor binding affinities that are sufficiently high (i.e., approx. 1-5 nmolar K_{d}'s) for further development as potential radiopharmaceuticals. These agonist conjugates are transported intracellularly after binding to cell surface GRP receptors and retained inside of the cells for extended time periods. In addition, in vivo studies in normal mice have shown that retention of the radioactive metal in the kidneys was low (i.e., <5%) with the majority of the radioactivity excreted into the urine.

According to one aspect of the present invention, there is provided a BBN conjugate consisting of essentially a radio-metal chelate covalently appended to the receptor binding region of BBN [e.g., BBN(8-14) or BBN(7-14)] to form radiolabeled BBN analogues that have high specific binding affinities with GRP receptors. These analogues are retained for long times inside of GRP expressing cancer cells. Furthermore, their clearance from the bloodstream, into the urine with minimal kidney retention, is efficient. Preferably, the radiometals are selected from ^{99m}Tc, ^{186/188}Re, ¹⁰⁵Rh, ¹⁵³Sm, ¹⁶⁶Ho, ⁹⁰Y, ¹⁹⁹Au, ¹⁷⁷Lu, ¹⁴⁹Pr, or ¹¹¹In, all of which hold the potential for diagnostic (i.e., ^{99m}Tc and ¹¹¹In) or therapeutic (i.e., ^{186/188}Re, ¹⁰⁵Rh, ¹⁵³Sm, ¹⁶⁶Ho, ⁹⁰Y, ¹⁹⁹Au, ¹⁷⁷Lu, ¹⁴⁹Pm, ¹⁶⁶Dy, ¹⁷⁵Yb, ^{117m}Sm and ¹¹¹In) utility in cancer patients [Schubiger et al, 1996; Eckelman, 1995; Troutner, 1978].

### SUMMARY OF THE INVENTION

In accordance with the present invention, there is provided a compound for use as a therapeutic or diagnostic radiopharmaceutical which includes a group which is capable of complexing a metal attached to a moiety capable of binding to a gastrin releasing peptide receptor.

Additionally, in accordance with the present invention, a method for treating a subject having a neoplastic disease which includes the step of administering to the subject an effective amount of a radiopharmaceutical having a metal chelated with a chelating group attached to a moiety capable of binding to a gastrin releasing peptide receptor on a cancer cell, subsequently intracellularly transported and residualized inside the cell, is disclosed.

Additionally, in accordance with the present invention, a method of forming a therapeutic or diagnostic compound including the step of reacting a metal synthon with a chelating group covalently linked with a moiety capable of binding a gastrin releasing peptide receptor is disclosed.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates a radiometal conjugate according to the present invention;

FIG. 2 is an ORTEP drawing of the {Rh[16]aneS₄-olCl₂}⁺, illustrating the crystal structure a Rhodium macrocycle;

FIG. 3 illustrates a coupling reaction wherein a spacer group is coupled to a bombesin agonist binding moiety;

FIG. 4 illustrates a coupling reaction for coupling a metal chelate to a peptide;

FIG. 5 illustrates several iodinated bombesin analogues including their IC₅₀'s;

FIG. 6 illustrates several tethered bombesin analogues;

FIG. 7 illustrates several [16]aneS₄ bombesin analogues;

FIG. 8 is a graph illustrating IC₅₀ analysis wherein %-1-125-BBN total uptake versus molar concentration of displacing ligand is shown;

FIG. 9 illustrates several Rhodium-[16]aneS₄ bombesin analogues;

FIG. 10 illustrates an HPLC chromatogram of Rhodium-BBN-37 wherein (A) illustrates ¹⁰⁵RhCl₂-BBN-37 and (B) illustrates RhCl₂-BBN-37;

FIG. 11 is a graph illustrating ¹²⁵I-Tyr⁴-bombesin internalization efflux from Swiss 3T3 cells;

FIG. 12 illustrates 1-125 bombesin internalization efflux in 1-125 free buffer wherein ¹²⁵I-Tyr⁴-BBN vs. ¹²⁵I-Lys³-BBN efflux from Swiss 3T3 cells is shown;

FIG. 13 is a graph illustrating the efflux of ¹⁰⁵Rh-BBN-37 from Swiss 3T3 cells;

FIG. 14 illustrates several ¹⁰⁵Rhodium bombesin analogues including their IC₅₀'s;

FIG. 15 is a graph illustrating ¹⁰⁵Rh-BBN-61 efflux from Swiss 3T3 cells;

FIG. 16 is a graph illustrating the efflux of ¹⁰⁵Rh-BBN-22 vs. ¹⁰⁵Rh-BBN-37 from Swiss 3T3 cells;

FIG. 17 are graphs illustrating Pancreatic CA cell binding wherein (A) illustrates the efflux ¹²⁵I-Tyr⁴-BBN from CF PAC1 cells and (B) illustrates the efflux of ¹⁰⁵Rh-BBN-37 from CF PAC1 cells;

FIG. 18 are graphs illustrating Prostate CA cell binding wherein (A) illustrates the efflux of ¹²⁵I-Tyr⁴-BBN from PC-3 cells and (B) illustrates the efflux of ¹⁰⁵Rh-BBN-37 from PC-3 cells;

FIG. 19 illustrates 5 [16]aneS₄ bombesin analogues which utilize amino acids as Linking Groups;

FIG. 20 illustrates 4 Rhodium-[16]aneS₄ bombesin analogues and IC₅₀ values obtained in 3 cell lines;

FIG. 21 illustrates 3 different N₃S-BFCA conjugates of BBN(7-14);

FIG. 22 illustrates on HPLC chromatogram of ^{99m}Tc-BBN-122;

FIG. 23 is a graph illustrating ^{99m}TC-BBN-122 internalization into human prostate cancer cells (PC-3 cells);

FIG. 24 is a graph illustrating ^{99m}Tc-BBN-122 internalization into human pancreatic tumor cells (CFPAC-1 cells);

FIG. 25 is a graph illustrating ^{99m}Tc-RP-414-BBN-42 retention in PC-3 prostate cancer cells;

FIG. 26 is a graph illustrating 99 mTc-42 retention in CFPAC-1 pancreatic cancer cells;

FIG. 27 illustrates further radiometal conjugates according to the present invention;

FIG. 28 are HPLC chromatograms of (a) DOTA-BBN[7-14]—NH₂ (λ=280 nm) (b) In-DOTA-BBN[7-14]NH₂ (λ=280 nm) and (c) “¹¹¹In-DOTA-BBN[7-14]NH₂ (radiometric);

FIG. 29 is a graph showing the competitive binding assay of In-DOTA-8-Aoc-BBN[7-14]NH₂ v. ¹²⁵I-Tyr⁴-BBN in PC-3 cells;

FIG. 30 is a graph showing the internalization of ¹¹¹In-DOTA-8-Aoc-BBN[7-14]NH₂ in PC-3 cells;

FIG. 31 is a graph showing the efflux of ¹¹¹In-DOTA-8-Aoc-BBN[7-14]NH₂ in PC-3 cells; and

FIG. 32 is illustrates radiometal conjugate according to the present invention.

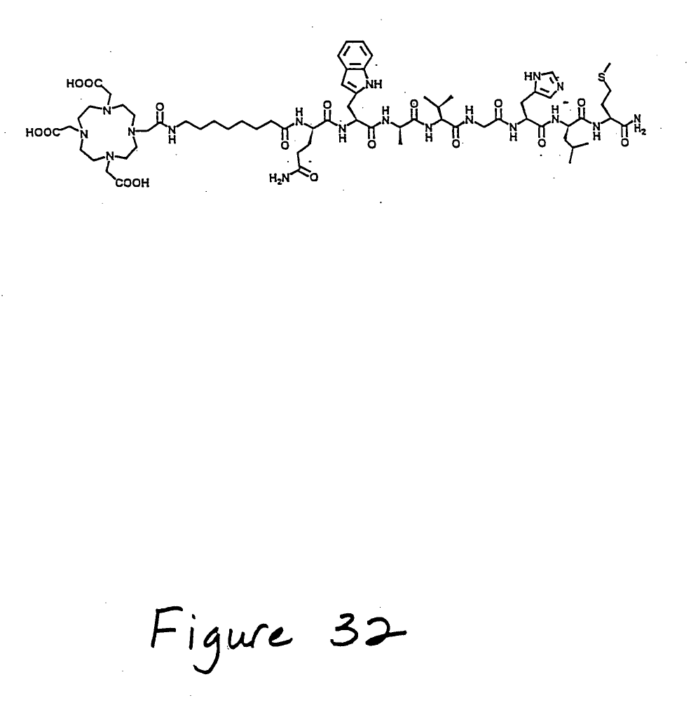

### DETAILED DESCRIPTION OF THE INVENTION

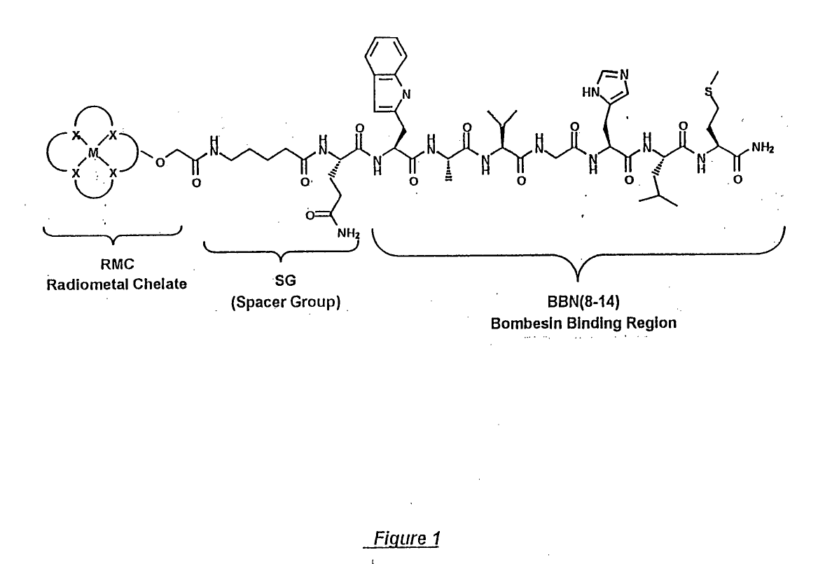

According to the present invention, compounds for use as diagnostic and/or therapeutic radiopharmaceuticals include a group capable of complexing a metal attached to a moiety capable of binding to a gastrin releasing peptide (GRP) receptor as shown in FIG. 1. These compounds can be prepared with either a diagnostic radiometal or a therapeutic radiometal thus affording utilities as either a diagnostic agent to identify cancerous tissues within the body using scintigraphic imaging techniques, or a therapeutic agent for the treatment and control of cancerous tissues. The moiety capable of specific binding to the GRP receptor is a GRP agonist. A GRP agonist activates or produces response by the GRP receptor upon interaction with the GRP receptor and is subsequently internalized inside of the cell by endocytosis. In contrast, a GRP antagonist counteracts the effect of an agonist and is not internalized inside of the cell.

More specifically, the GRP agonist for the purpose of this invention is a compound such as selected amino acid sequences or peptidomimetics which are internalized or residualized following binding with high affinity and selectivity to GRP receptors and that can be covalently linked to the metal complexing group. Many examples of specific modifications of the BBN(7-14) or BBN(8-14) that can be made to produce sequences with high antagonistic and agonistic binding affinity for GRP repectors have been reported by numerous investigations [Davis et al., 1992; Hoffken, 1994; Moody et al., 1996; Coy et al., 1988; Cai et al., 1994; Moody et al., 1995; Leban et al., 1994; Cai et al., 1992].

In a preferred embodiment of the present invention, the metal complexing group or moiety is a chelating agent or chelator which complexes to metals such as ¹⁰⁵Rh—, ^{186/188}Re—, ^{99m}Tc ¹⁵³Sm, ¹⁶⁶Ho, ⁹⁰Y, ¹¹¹In, ¹⁷⁷Lu, ¹⁴⁹Pm, ¹⁴⁹Sm or ¹⁹⁹Au. The chelating agent or chelator is attached or bound to the GRP agonist “binding region” through a spacer to produce a conjugate that retains its capability for high affinity and specific binding to GRP receptors.

In a more preferred embodiment of the present invention, the GRP agonist is a bombesin (BBN) analogue and/or a derivative thereof. The BBN derivative or analog thereof preferably contains either the same primary structure of the BBN binding region [i.e., BBN(8-14) or BBN(7-14)] or similar primary structures, with specific amino acid substitutions, that will specifically bind to GRP receptors with better or similar binding affinities as BBN alone (i.e., K_{d}≅1-5 nmolar) Compounds containing this BBN binding region (or binding moiety), when covalently linked to other groups (e.g., a radiometal chelate), are also referred to as BBN conjugates.

In general, the compounds of the present invention have a structure of the general formula:
X—Y—B
wherein X is a group capable of complexing a metal, such as a radiometal; Y is a covalent bond on a spacer group; and B is a bombesin agonist binding moiety.

The metal bound to the metal complexing group can be any suitable metal chosen for a specific therapeutic or diagnostic use including transition metals, lanthanides, auger electron emitting isotopes, a, 0 or y emitting isotopes. Preferably, the metal is a radiometal such as ¹⁰⁵Rh—, ^{99m}Tc—, ^{186/188}Re, ¹⁵³Sm—, ¹⁶⁶Ho—, ¹¹¹In, ⁹⁰Y—, ¹⁷⁷Lu, ¹⁴⁹Pm, ¹⁵³Sm, and ¹⁹⁹Au— whose chelates can be covalently linked (i.e., conjugated) to the specific BBN binding region via the N-terminal end of the primary binding sequence (e.g., BBN-8 or Trp⁸) as shown in FIG. 1.

In a preferred embodiment, the radiometal complexes are positioned by being spaced apart from or remotely from the amino acid Trp⁸ by the spacer groups. The spacer groups can include a peptide (i.e., ≧1 amino acid in length), a hydrocarbon spacer of C₁-C₁₀ or a combination of thereof. Preferably, the hydrocarbon spacer is a C₃-C₉ group. The resulting radio-labeled BBN conjugates retain high binding affinity and specificity for GRP receptors and are subsequently internalized inside of the cell.

The BBN conjugates can further incorporate a spacer group or component to couple the binding moiety to the metal chelator (or metal binding backbone) while not adversely affecting either the targeting function of the BBN-binding moiety or the metal complexing function of the metal chelating agent.

The term “spacer group” or “linker” refers to a chemical group that serves to couple the BBN binding moiety to the metal chelator while not adversely affecting either the targeting function of the BBN binding moiety or the metal complexing function of the metal chelator. Suitable spacer groups include peptides (i.e., amino acids linked together) alone, a non-peptide group (e.g., hydrocarbon chain) or a combination of an amino acid sequence and a non-peptide spacer. The type of spacer group used in most of the experimental studies described below in the Examples section were composed of a combination of L-glutamine and hydrocarbon spacers. A pure peptide spacer could consist of a series of amino acids (e.g., diglycine, triglycine, gly-gly-glu, gly-ser-gly, etc.), in which the total number of atoms between the N-terminal residue of the BBN binding moiety and the metal chelator in the polymeric chain is ≦12 atoms.

The spacer can also include a hydrocarbon chain [i.e., R₁—(CH₂)ₙ—R₂] wherein n is 0-10, preferably n=3 to 9, R₁ is a group (e.g., H₂N—, HS—, —COOH) that can be used as a site for covalently linking the ligand backbone or the preformed metal chelator or metal complexing backbone; and R₂ is a group that is used for covalent coupling to the N-terminal NH₂-group of the BBN binding moiety (e.g., R₂ is an activated COOH group). Several chemical methods for conjugating ligands (i.e., chelators) or preferred metal chelates to biomolecules have been well described in the literature [Wilbur, 1992; Parker, 1990; Hermanson, 1996; Frizberg et al., 1995]. One or more of these methods could be used to link either the uncomplexed ligand (chelator) or the radiometal chelate to the spacer group or to link the spacer group to the BBN(8-14) derivatives.

These methods include the formation of acid anhydrides, aldehydes, arylisothiocyanates, activated esters, or N-hydroxysuccinimides [Wilbur, 1992; Parker, 1990; Hermanson, 1996; Frizberg et al., 1995].

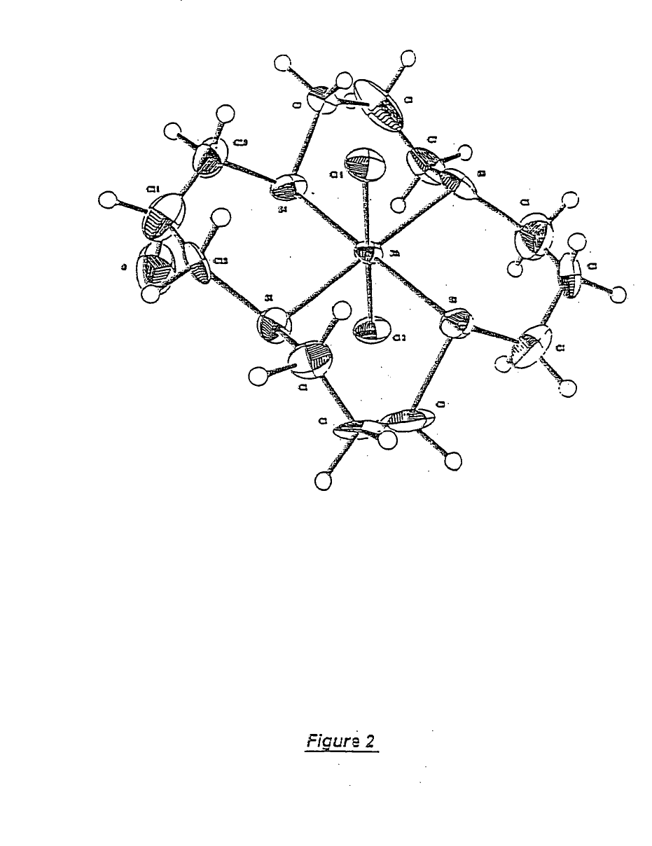

The term “metal complexing chelator” refers to a molecule that forms a complex with a metal atom that is stable under physiological conditions. That is, the metal will remain complexed to the chelator backbone in vivo. More particularly, a metal complexing chelator is a molecule that complexes to a radionuclide metal to form a metal complex that is stable under physiological conditions and which also has at least one reactive functional group for conjugation with the BBN agonist binding moiety. Metal complexing chelators can include monodentate and polydentate chelators [Parker, 1990; Frizberg et al., 1995; Lister-James et al., 1997; Li et al., 1996b; Albert et al., 1991; Pollak et al., 1996; de Jong et al., 1997; Smith et al., 1997] and include the DOTA chelators discussed in more detail below. Metal complexing chelators include tetradentate metal chelators which can be macrocyclic and have a combination of four nitrogen and/or sulfur metal-coordinating atoms [Parker et al., 1990; Li et al., 1996b] and are designated as N₄, S₄, N₃S, N₂S₂, NS₃, etc. as shown in FIG. 2. A number of suitable multidentate chelators that have been used to conjugate proteins and receptor-avid molecules have been reported [Frizberg et al., 1995; Lister-James et al., 1997; Li et al., 1996b; Albert et al., 1991; Pollak et al., 1996; de Jong et al., 1997] and include the DOTA chelators discussed in more detail below. These multidentate chelators can also incorporate other metal-coordinating atoms such as oxygen and phosphorous in various combinations. The metal binding complexing moiety can also include “3+1” chelators [Seifert et al., 1998].

For diagnostic purposes, metal complexing chelators preferably include chelator backbones for complexing the radionuclide metals ^{99m}Tc and ¹¹¹In. For therapeutic purposes, metal complexing chelators preferably include chelator backbones that complex the beta particle emitting radionuclide metals including ¹⁰⁵Rh, ^{186/188}Re ¹⁵³Sm, ⁹⁰Y, ¹⁶⁶Ho, ¹⁹⁹Au, ¹⁷⁷Lu, In, Dy, Yb and ¹⁴⁹Pm [Schubiger et al., 1996; Hoffken, 1994].

As was briefly described above, the term “bombesin agonist” or “BBN agonist” refers to compounds that bind with high specificity and affinity to GRP receptors, and upon binding to the GRP receptor, are intracellularly internalized. Suitable compounds include peptides, peptidomimetics and analogues and derivatives thereof. In particular, previous work has demonstrated that the region on the BBN peptide structure required for binding to GRP receptors spans from residue 8 through 14 [Davis et al., 1992; Hoffken, 1994; Moody et al., 1996; Coy, 1988; Cai et al., 1994]. The presence of methionine (Met) at position BBN-14 will generally confer agonistic properties while the absence of this residue at BBN-14 generally confers antagonistic properties [Hoffken, 1994].

It is well documented in the art that there are a few and selective number of specific amino acid substitutions in the BBN (8-14) binding region (e.g., D-Ala¹¹ for L-Gly¹¹ or D-Trp⁸ for L-Trp⁸), which can be made without decreasing binding affinity [Leban et al., 1994; Qin et al., 1994; Jensen et al., 1993]. In addition, attachment of some amino acid chains or other groups to the N-terminal amine group at position BBN-8 (i.e., the Trp⁸ residue) can dramatically decrease the binding affinity of BBN analogues to GRP receptors [Davis et al., 1992; Hoffken, 1994; Moody et al., 1996; Coy, et al., 1988; Cai et al., 1994]. In a few cases, it is possible to append additional amino acids or chemical moieties without decreasing binding affinity. The effects of conjugating various side chains to BBN-8 on binding affinity, therefore, is not predicable.

The BBN conjugates of the present invention can be prepared by various methods depending upon the selected chelator. The peptide portion of the conjugate can be most conveniently prepared by techniques generally established and known in the art of peptide synthesis, such as the solid-phase peptide synthesis (SPPS) approach. Solid-phase peptide synthesis (SPPS) involves the stepwise addition of amino acid residues to a growing peptide chain that is linked to an insoluble support or matrix, such as polystyrene. The C-terminal residue of the peptide is first anchored to a commercially available support with its amino group protected with an N-protecting agent such as a t-butyloxycarbonyl group (tBoc) or a fluorenylmethoxycarbonyl (FMOC) group. The amino protecting group is removed with suitable deprotecting agents such as TFA in the case of tBOC or piperidine for FMOC and the next amino acid residue (in N-protected form) is added with a coupling agent such as dicyclocarbodiimide (DCC). Upon formation of a peptide bond, the reagents are washed from the support. After addition of the final residue, the peptide is cleaved from the support with a suitable reagent such as trifluoroacetic acid (TFA) or hydrogen fluoride (HF).

The spacer groups and chelator components are then coupled to form a conjugate by reacting the free amino group of the Trp⁸ residue of the BBN binding moiety with an appropriate functional group of the chelator, metal chelator or spacer group, such as a carboxyl group or activated ester.

The BBN conjugate can also incorporate a metal complexing chelator backbone that is peptidic and compatible with solid-phase peptide synthesis. In this case, the chelator backbone can be added to the BBN binding moiety in the same manner as described above or, more conveniently, the metal complexing chelator backbone coupled to the BBN binding moiety can be synthesized in toto starting from the C-terminal residue of the peptide and ending with the N-terminal residue of the metal complexing chelator structure.

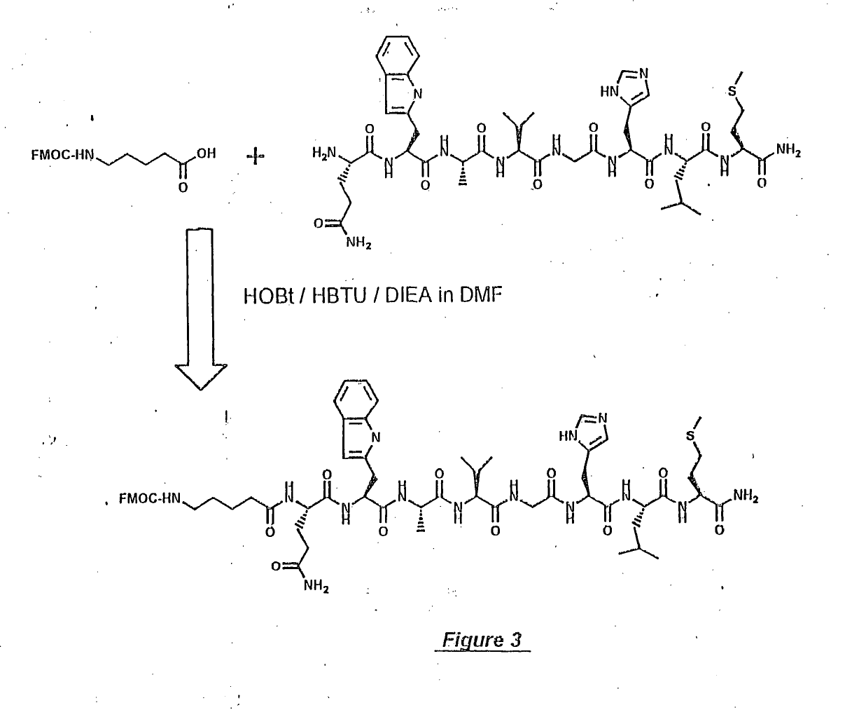

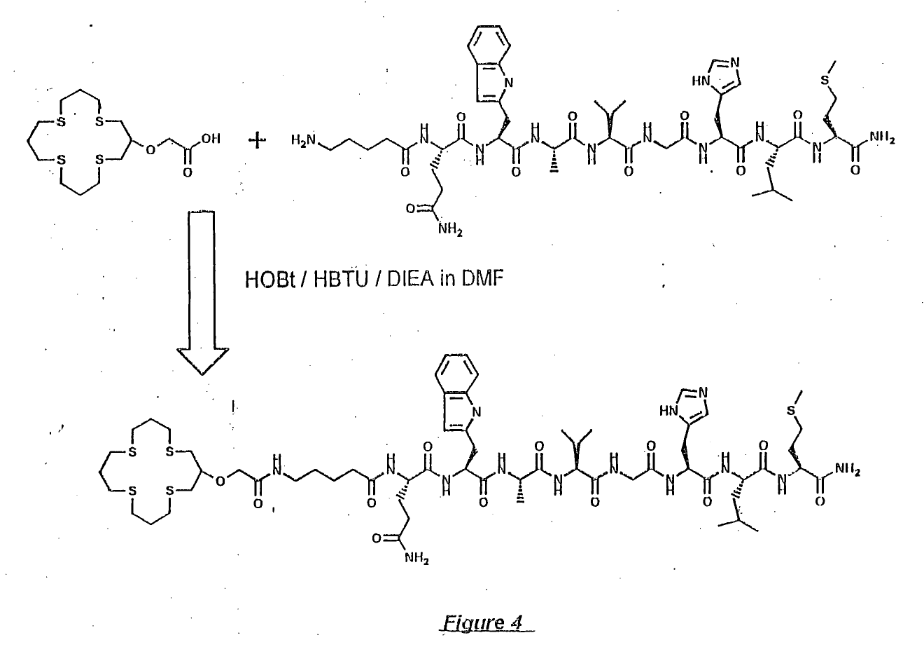

The chelator backbones used in accordance with the present invention are commercially available or they could be made by methods similar to those outlined in the literature [Frizberg et al., 1995; Lister-James et al., 1997; Li et al., 1996b; Albert et al., 1991; Pollak et al., 1996; de Jong et al., 1997; Smith et al., 1997; Seifert et al., 1998]. Attachment of the spacer groups to functionalizable atoms appended to the ligand backbone can be performed by standard methods known to those skilled in the art. For example, the HOBt/HBTU activated —COOH group on 5-aminovaleric acid (5-AVA) was reacted with the N-terminal amine on Gln⁷ to produce an amide linkage as shown in FIG. 3. Similarly, the —COOH group attached to the characterized [16]aneS₄ ligand was conjugated to the amine group on the hydrocarbon spacer (shown below) by reaction of the HOBt/HBTU activated carboxyl group appended to the [16]aneS₄ macrocycle with the terminal amine group on 5-AVA to form BBN-37 as shown in FIG. 4. Other standard conjugation reactors that produce covalent linkages with amine groups can also be used [Wilbur, 1992; Parker, 1990].

The chelating framework, conjugated via Trp⁸, that complexes the radiometals should form a 1:1 chelator to metal ratio. Since ^{99m}Tc has a short half-life (6 hour) and is a diagnostic radionuclide, the method of forming the ^{99m}Tc-BBN analogues should permit complexation (either directly or by transmetallation) of ^{99m}Tc to the conjugated chelating framework in a one-step, high yield reaction (exemplified below in the Experimental Section).

In contrast, the longer half lives of the therapeutic radionuclides (e.g., ¹⁰⁵Rh, ^{186/188}Re, ¹⁵³Sm, ¹⁶⁶Ho, ⁹⁰Y, ¹⁷⁷Lu, ¹⁴⁹Pm, ¹⁹⁹Au, ¹¹¹In, ¹⁷⁷Lu) permit formation of the corresponding radiolabeled BBN analogues by either a one step high yield complexation step or by performing a ¹⁰⁵Rh—, ^{186/188}Re—, ¹⁵³Sm, ¹⁶⁶Ho, ⁹⁰Y, ¹⁷⁷Lu, ¹¹¹In or ¹⁴⁹Au chelate synthon followed by conjugation of the preformed complex to the N-terminal end of the BBN binding moiety. In all cases, the resulting specific activity of the final radiolabeled BBN derivative must be high (i.e., >1Ci/μmole).

Re- and Tc-Conjugates

Re and Tc are both in row VIIB of the Periodic Table and they are chemical congeners. Thus, for the most part, the complexation chemistry of these two metals with ligand frameworks that exhibit high in vitro and in vivo stabilities are the same [Eckelman, 1995]. Many ^{99m}Tc or ^{186/188}Re complexes, which are employed to form stable radiometal complexes with peptides and proteins, chelate these metals in their +5 oxidation state [Lister-James et al., 1997]. This oxidation state makes it possible to selectively place ^{99m}Tc- or ^{186/188}Re into ligand frameworks already conjugated to the biomolecule, constructed from a variety of ^{99m}Tc(V) and/or ^{186/188}Re(V) weak chelates (e.g., ^{99m}Tc-glucoheptonate, citrate, gluconate, etc.) [Eckelman, 1995; Lister-James et al., 1997; Pollak et al., 1996]. Tetradentate ligand frameworks have been shown to form well-defined, single chemical species in high specific activities when at least one thiol group or at least one hydroxymethylene phosphine group is present on the ligand backbone [Smith et al., 1997].

Ligands which form stable Tc(V) or Re(V) tetradentate complexes containing, but not limited to, amino N-atoms, amido-N-atoms, carboxy-O-atoms and thioether-S-atoms, are donor atoms that can also be present [Eckelman, 1995; Fritzberg et al., 1992; Parker, 1990; Frizberg et al., 1995; Pollak et al., 1996; Seifert et al., 1998]. Depending upon the mix of donor atoms (groups), the overall complex charge normally ranges from −1 to +1.

Incorporation of the metal within the conjugate can be achieved by various methods commonly known in the art of coordination chemistry. When the metal is technetium-99m, the following general procedure can be used to form a technetium complex. A peptide-chelator conjugate solution is formed by initially dissolving the conjugate in water or in an aqueous alcohol such as ethanol. The solution is then degassed to remove oxygen. When an —SH group is present in the peptide, the thiol protecting group(s) are removed with a suitable reagent, for example with sodium hydroxide, and are then neutralized with an organic acid such as acetic acid (pH 6.0-6.5). In the labeling step, sodium pertechnetate obtained from a molybdenum generator is added to a solution of the conjugate with a sufficient amount of a reducing agent, such as stannous chloride, to reduce technetium and is either allowed to stand at room temperature or is heated. The labeled conjugate can be separated from the contaminants ^{99m}TcO₄⁻ and colloidal ^{99m}TcO₂ chromatographically, for example with a C-18 Sep Pak cartridge [Millipore Corporation, Waters Chromatography Division, 34 Maple Street, Milford, Mass. 01757].

In an alternative method, the labeling can be accomplished by a transchelation reaction. The technetium source is a solution of technetium complexed with labile ligands facilitating ligand exchange with the selected chelator. Examples of suitable ligands for transchelation includes tartrate, citrate, gluconate, and heptagluconate. It will be appreciated that the conjugate can be labeled using the techniques described above, or alternatively, the chelator itself may be labeled and subsequently coupled to the peptide to form the conjugate; a process referred to as the “prelabeled chelate” method.

When labeled with diagnostically and/or therapeutically useful metals, peptide-chelator conjugates or pharmaceutically acceptable salts, esters, amides, and prodrugs of the present invention can be used to treat and/or detect cancers, including tumors, by procedures established in the art of radiodiagnostics and radiotherapeutics. [Bushbaum, 1995; Fischman et al., 1993; Schubiger et al., 1996; Lowbertz et al., 1994; Krenning et al., 1994]. A conjugate labeled with a radionuclide metal, such as technetium-99m, can be administered to a mammal, including human patients or subjects, by intravenous or intraperitoneal injection in a pharmaceutically acceptable carrier and/or solution such as salt solutions like isotonic saline. The amount of labeled conjugate appropriate for administration is dependent upon the distribution profile of the chosen conjugate in the sense that a rapidly cleared conjugate may be administered in higher doses than one that clears less rapidly. Unit doses acceptable for Tc-99m imaging radiopharmaceuticals are in the range of about 5-40 mCi for a 70 kg individual. In vivo distribution and localization can be tracked by standard scintigraphic techniques at an appropriate time subsequent to administration; typically between thirty minutes and 180 minutes depending upon the rate of accumulation at the target site with respect to the rate of clearance at non-target tissue.

The compounds of the present invention can be administered to a patient alone or as part of a composition that contains other components such as excipients, diluents, radical scavengers, stabilizers, and carriers, all of which are well-known in the art. The compounds can be administered to patients either intravenously or intraperitoneally.

There are numerous advantages associated with the present invention. The compounds made in accordance with the present invention form stable, well-defined ^{99m}Tc or ^{186/188}Re conjugate analogues of BBN agonists. Similar BBN agonist analogues can also be made by using appropriate chelator frameworks for the respective radiometals, to form stable-well-defined products labeled with ¹⁵³Sm, ⁹⁰Y, ¹⁶⁶Ho, ¹⁰⁵Rh, ¹⁹⁹Au, ¹⁴⁹Pm, ¹⁷⁷Lu, or ¹¹¹In. The radiolabeled BBN agonist conjugates, selectively bind to neoplastic cells expressing GRP receptors, become internalized, and are retained in the tumor cells for extended time periods. Incorporating the spacer group between the metal chelator and the BBN agonist binding moiety maximizes the uptake and retention of the radioactive metal inside of the neoplasts or cancer cells. The radioactive material that does not reach (i.e., does not bind) the cancer cells is preferentially excreted efficiently into the urine with minimal radiometal retention in the kidneys.

Radiotherapeutics

The diagnostic application of these compounds can be as a first line diagnostic screen for the presence of neoplastic cell using scintigraphic imaging, as an agent for targeting neoplastic tissue using hand held radiation detection instrumentation in the field of radioimmuno guided surgery (RIGS), as a means to obtain dosimetry data prior to administration of the matched pair therapeutic compound, and as a means to assess GRP receptor population as a function of treatment over time.

The therapeutic application of these compounds can be defined either as an agent that will be used as a first line therapy in the treatment of cancer, as combination therapy where these radiolabeled agents could be utilized in conjunction with adjuvant chemotherapy, and as the matched pair therapeutic agent. The matched pair concept refers to one compound which can serve as both a diagnostic and a therapeutic agent depending on the radiometal with the appropriate chelate selected and can be understood in connection with the data set forth below.

Radioisotope therapy involves the administration of a radiolabeled compound in sufficient quantity to damage or destroy the targeted tissue. After administration of the compound (by e.g. intravenous or intraperitonal injection), the radiolabeled pharmaceutical localizes preferentially at the disease site (in this instance, tumor tissue that expresses the GRP-receptor). Once localized, the radiolabeled compound then damages or destroys the diseased tissue with the energy that is released during the radioactive decay of the isotope that is administered.

The design of a successful radiotherapeutic involves several critical factors:
1. selection of an appropriate targeting group to deliver the radioactivity to the disease site;
2. selection of an appropriate radionuclide that releases sufficient energy to damage that disease site, without substantially damaging adjacent normal tissues; and
3. selection of an appropriate combination of the targeting group and the radionuclide without adversely affecting the ability of this conjugate to localize at the disease site. For radiometals, this often involves a chelating group that coordinates tightly to the radionuclide, combined with a linker that couples said chelate to the targeting group, and that affects the overall biodistribution of the compound to maximize uptake in target tissues and minimizes uptake in normal, non-target organs.

The present invention provides radiotherapeutic agents that satisfy all three of the above criteria, through proper selection of targeting group, radionuclide, metal chelate and linker.

Radiotherapeutic agents may contain a chelated 3+metal ion from the class of elements known as the lanthanides (elements of atomic number 57-71) and their analogs (i.e. M³⁺ metals such as yttrium and indium). Typical radioactive metals in this class include the isotopes 90—Yttrium, 111-Indium, 149-Promethium, 153-Samarium, 166-Dysprosium, 166-Holmium, 175-Ytterbium, and 177-Lutetium. All of these metals (and others in the lanthanide series) have very similar chemistries, in that they remain in the +3 oxidation state, and prefer to chelate to ligands that bear hard (oxygen/nitrogen) donor atoms, as typified by derivatives of the well known chelate DTPA (Diethylenetriaminepentaacetic acid) and polyaza-polycarboxylate macrocycles such as DOTA (1,4,7,10-tetrazacyclododecane-N,N′,N″,N′″-tetraacetic acid and its close analogs. The structures of these chelating ligands, in their fully deprotonated form are shown below.

| DTPA | DOTA |
|---|---|
| 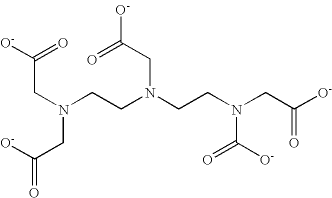 | 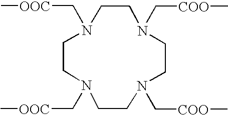 |

These chelating ligands encapsulate the radiometal by binding to it via multiple nitrogen and oxygen atoms, thus preventing the release of free (unbound) radiometal into the body. This is important, as in vivo dissociation of +3 radiometals from their chelate can result in uptake of the radiometal in the liver, bone and spleen [Brechbiel M W, Gansow O A, “Backbone-substituted DTPA ligands for ⁹⁰Y radioimmunotherapy”, Bioconj. Chem. 1991; 2: 187-194; Li, W P, Ma D S, Higginbotham C, Hoffman T, Ketring A R, Cutler C S, Jurisson, S S, “Development of an in vitro model for assessing the in vivo stability of lanthanide chelates.” Nucl. Med. Biol. 2001; 28(2): 145-154; Kasokat T, Urich K. Arzneim.-Forsch, “Quantification of dechelation of gadopentetate dimeglumine in rats”. 1992; 42(6): 869-76]. Unless one is specifically targeting these organs, such non-specific uptake is highly undesirable, as it leads to non-specific irradiation of non-target tissues, which can lead to such problems as hematopoietic suppression due to irradiation of bone marrow.

For radiotherapy applications, forms of the DOTA chelate [Tweedle M F, Gaughan G T, Hagan J T, “1-Substituted-1,4,7-triscarboxymethyl-1,4,7,10-tetraazacyclododecane and analogs.” U.S. Pat. No. 4,885,363, Dec. 5, 1989] are particularly preferred, as the DOTA chelate is expected to de-chelate less in the body than DTPA or other linear chelates.

General methods for coupling DOTA-type macrocycles to targeting groups through a linker (e.g. by activation of one of the carboxylates of the DOTA to form an active ester, which is then reacted with an amino group on the linker to form a stable amide bond), are known to those skilled in the art. (See e.g. Tweedle et al. U.S. Pat. No. 4,885,363). Coupling can also be performed on DOTA-type macrocycles that are modified on the backbone of the polyaza ring.

The selection of a proper nuclide for use in a particular radiotherapeutic application depends on many factors, including:

a. Physical half-life—This should be long enough to allow synthesis and purification of the radiotherapeutic construct from radiometal and conjugate, and delivery of said construct to the site of injection, without significant radioactive decay prior to injection. Preferably, the radionuclide should have a physical half-life between about 0.5 and 8 days.

b. Energy of the emission(s) from the radionuclide—Radionuclides that are particle emitters (such as alpha emitters, beta emitters and Auger electron emitters) are particularly useful, as they emit highly energetic particles that deposit their energy over short distances, thereby producing highly localized damage. Beta emitting radionuclides are particularly preferred, as the energy from beta particle emissions from these isotopes is deposited within 5 to about 150 cell diameters. Radiotherapeutic agents prepared from these nuclides are capable of killing diseased cells that are relatively close to their site of localization, but cannot travel long distances to damage adjacent normal tissue such as bone marrow.

c. Specific activity (i.e. radioactivity per mass of the radionuclide)—Radionuclides that have high specific activity (e.g. generator produced 90—Y, 111-In, 177—Lu) are particular preferred. The specific activity of a radionuclide is determined by its method of production, the particular target that is used to produce it, and the properties of the isotope in question.

Many of the lanthanides and lanthanoids include radioisotopes that have nuclear properties that make them suitable for use as radiotherapeutic agents, as they emit beta particles. Some of these are listed in the table below.

| | | | | Approximate |
|---|---|---|---|---|
| | | | | range of b- |
| | Half-Life | Max □-energy | Gamma energy | particle |
| Isotope | (days) | (MeV) | (keV) | (cell diameters) |
| 149-Pm | 2.21 | 1.1 | 286 | 60 |
| 153-Sm | 1.93 | 0.69 | 103 | 30 |
| 166-Dy | 3.40 | 0.40 | 82.5 | 15 |
| 166-Ho | 1.12 | 1.8 | 80.6 | 117 |
| 175-Yb | 4.19 | 0.47 | 396 | |
| 177-Lu | 6.71 | 0.50 | 208 | 20 |
| 90-Y | 2.67 | 2.28 | — | 150 |
| 111-In | 2.810 | Auger electron | 173, 247 | <5 μm |
| | | emitter | | |

Pm: promethium,
Sm: samarium,
Dy: dysprosium,
Ho: holmium,
Yb: ytterbium,
Lu: lutetium,
Y: yttrium,
In: Indium

Methods for the preparation of radiometals such as beta-emitting lanthanide radioisotopes are known to those skilled in the art, and have been described elsewhere [e.g. Cutler C S, Smith C J, Ehrhardt G J.; Tyler T T, Jurisson S S, Deutsch E. “Current and potential therapeutic uses of lanthanide radioisotopes.” Cancer Biother. Radiopharm. 2000; 15(6): 531-545]. Many of these isotopes can be produced in high yield for relatively low cost, and many (e.g. 90—Y, 149—Pm, 177—Lu) can be produced at close to carrier-free specific activities (i.e. the vast majority of atoms are radioactive). Since non-radioactive atoms can compete with their radioactive analogs for binding to receptors on the target tissue, the use of high specific activity radioisotope is important, to allow delivery of as high a dose of radioactivity to the target tissue as possible.

Radiotherapeutic derivatives of the invention containing beta-emitting isotopes of rhenium (186—Re and 188—Re) are also particularly preferred.

Proper dose schedules for the radiotherapeutic compounds of the present invention are known to those skilled in the art. The compounds can be administered using many methods which include, but are not limited to, a single or multiple IV or IP injections, using a quantity of radioactivity that is sufficient to cause damage or ablation of the targeted GRP-R bearing tissue, but not so much that substantive damage is caused to non-target (normal tissue). The quantity and dose required is different for different constructs, depending on the energy and half-life of the isotope used, the degree of uptake and clearance of the agent from the body and the mass of the tumor. In general, doses can range from a single dose of about 30-50 mCi to a cumulative dose of up to about 3 Curies.

The radiotherapeutic compositions of the invention can include physiologically acceptable buffers, and can require radiation stabilizers to prevent radiolytic damage to the compound prior to injection. Radiation stabilizers are known to those skilled in the art, and may include, for example, para-aminobenzoic acid, ascorbic acid, gentistic acid and the like.

The following examples are presented to illustrate specific embodiments and demonstrate the utility of the present invention.

### EXPERIMENTAL SECTION

### Example 1

### Synthesis and In Vitro Binding Assessment of Synthetic BBN Analogues Employing Hydrocarbon Chain Spacers

A. Synthesis:

Many BBN analogues were synthesized by Solid Phase Peptide Synthesis (SPPS). Each peptide was prepared by SPPS using an Applied Biosystems Model 432A peptide synthesizer. After cleavage of each BBN analogue from the resin using Trifluoracetic acid (TFA), the peptides were purified by C18 reversed-phase HPLC using a Vydac HS54 column and CH₃CN/H₂O containing 0.1% TFA as the mobile phase. After collection of the fraction containing the desired BBN peptide (approx. 80-90% yield in most cases), the solvent was evaporated. The identity of each BBN peptide was confirmed by FAB-mass spectrometry, Department of Chemistry—Washington University, St. Louis, Mo.

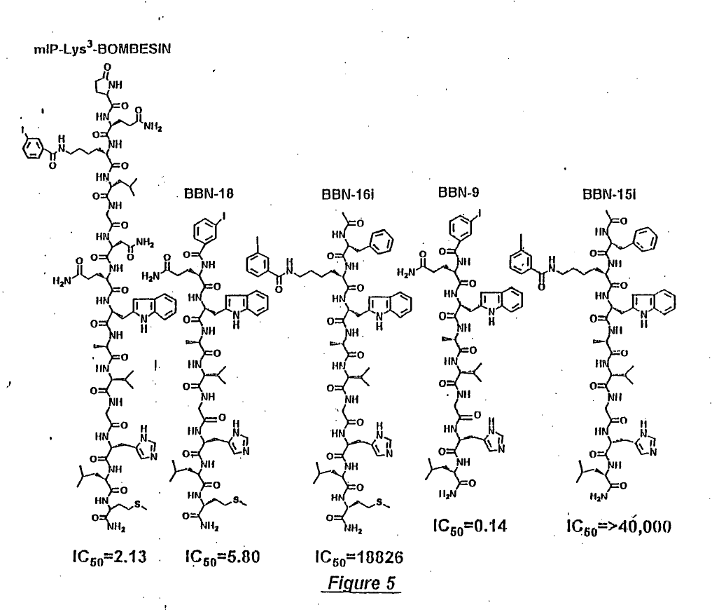

Various amino acid sequences (in some cases including different chemical moieties) were conjugated to the N-terminal end of the BBN binding region (i.e., to BBN-8 or Trp8). BBN analogue numbers 9, 15, 15i, 16, 16i and 18 were synthesized as examples of N-terminal modified peptides as shown in FIG. 5.

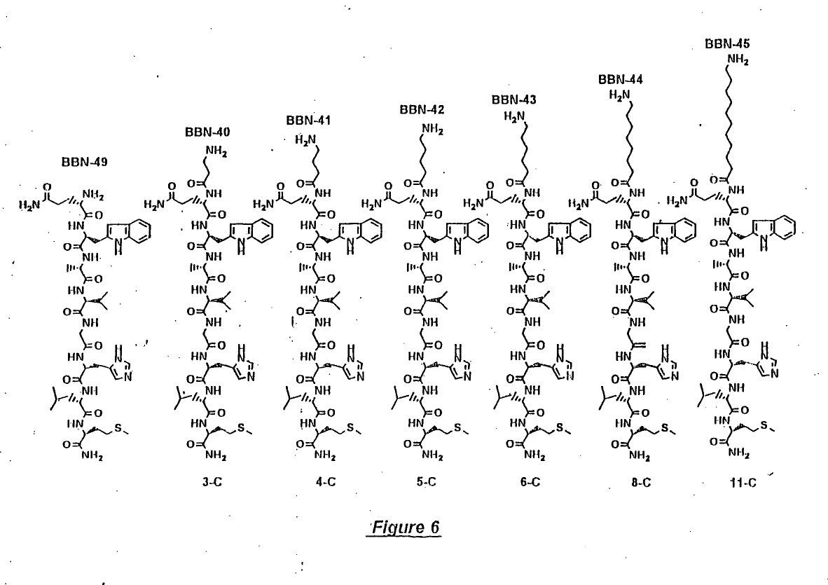

Various tethered N-terminal (via Trp8) BBN analogues were also synthesized by SPPS as exemplified by BBN-40, BBN41, BBN42, BBN43, BBN44, BBN45, and BBN49 as shown in FIG. 6. In these particular tethered peptides, a Glu residue was attached to Trp8 followed by attachment of fmoc protected terminal amine groups separated from a —COOH group by 3-, 4-, 5-, 6-, 8- and 11-carbon chain (CH) spacers (FIG. 6). These fmoc protected acids were added as the terminal step during the SPPS cycle. As described previously, each of the BBN analogues was purified by reversed-phase HPLC and characterized by high resolution Mass Spectroscopy. Peptide 49 employed only glutamine as the spacer group.

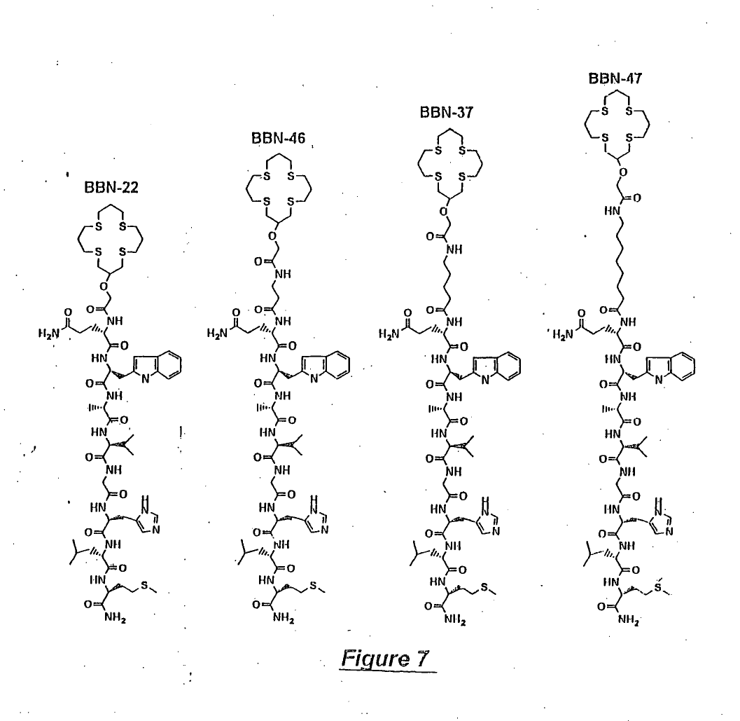

The [16]aneS4 macrocyclic ligand was conjugated to selected tethered BBN analogues shown in FIG. 6. The —OCH₂COOH group on the [16]aneS4 macrocycle derivative was activated via HOBt/HBTU so that it efficiently formed an amide bond with the terminal NH₂ group on the spacer side arm (following deprotection). The corresponding [16]aneS₄ tethered BBN derivatives were produced and examples of 4 of these derivatives (i.e., BBN-22, -37, -46 and -47) are shown in FIG. 7. As previously described, each [16]aneS₄ BBN derivative was purified by reversed phase HPLC and characterized by FAB Mass Spectroscopy.

B. In Vitro Binding Affinities

The binding affinities of the synthetic BBN derivatives were assessed for GRP receptors on Swiss 3T3 cells and, in some cases, on a variety of human cancer cell lines, that express GRP receptors. The IC₅₀ value of each derivative was determined relative to (i.e., in competition with) ¹²⁵I-Tyr₄-BBN (the Kd for ¹²⁵I-Tyr₄-BBN for GRP receptors in Swiss 3T3 cells is reported to be 1.6+0.4 nM) [Zueht et al., 1991]. The cell binding assay methods used to measure the IC₅₀'s is standard and used techniques previously reported [Jensen et al., 1993; Cai et al., 1994; Cai et al., 1992]. The methods used for determining IC₅₀'s for all GRP receptor binding compounds on all cell lines was similar. The specific method used to measure IC₅₀'s on Swiss 3T3 cells is briefly described as follows:

Swiss 3T3 mouse fibroblasts are grown to confluence in 48 well microtiter plates. An incubation media was prepared consisting of HEPES (11.916 g/l), NaCl (7.598 g/l), KCl (0.574 g/l), MgCl2 (1.106 g/l), EGTA (0.380 g/l), BSA (5.0 g/l), chymostatin (0.002 g/l), soybean trypsin inhibitor (0.200 g/l), and bacitracin (0.050 g/l). The growth media was removed, the cells were washed twice with incubation media, and incubation media was returned to the cells. ¹²⁵I-Tyr4-BBN (0.01 uCi) was added to each well in the presence of increasing concentrations of the appropriate competitive peptide. Typical concentrations of displacing peptide ranged from 10-12 to 10-5 moles of displacing ligand per well. The cells were incubated at 37° C. for forty minutes in a 95% O2/5% CO₂ humidified environment. At forty minutes post initiation of the incubation, the medium was discarded, and the cells were washed twice with cold incubation media. The cells were harvested from the wells following incubation in a trypsin/EDTA solution for five minutes at 37° C. Subsequently, the radioactivity, per well, was determined and the maximum % total uptake of the radiolabeled peptide was determined and normalized to 100%.

C. Results of Binding Affinity Measurements:

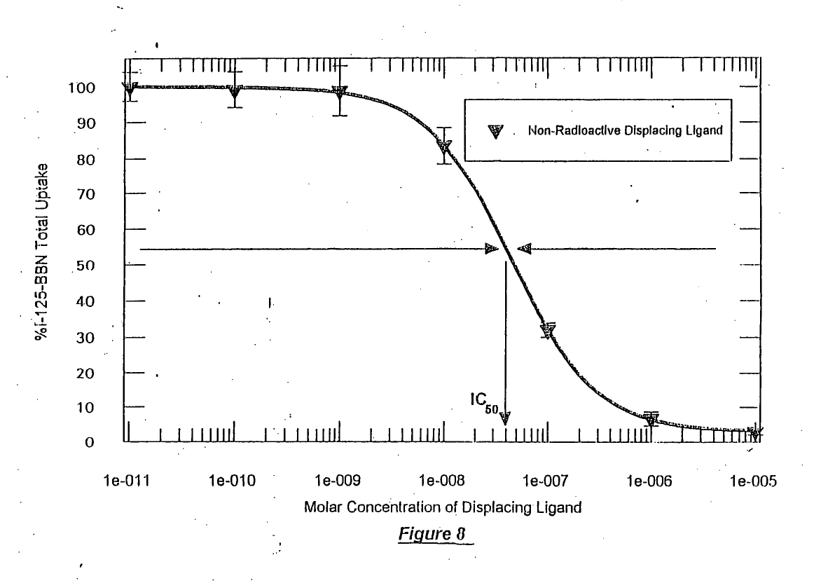

The IC₅₀ values measured for the BBN derivatives synthesized in accordance with this invention showed that appending a peptide side chain and other moieties via the N-terminal BBN-8 residue (i.e., Trp⁸) produced widely varying IC₅₀ values. For example, see IC₅₀ values shown for BBN 11, 15i, 16i, and 18 in FIGS. 5 and 8. The observations are consistent with previous reports showing highly variable IC₅₀ values when derivatizing BBN(8-13) or BBN(8-14) with a predominantly short chain of amino acid residues [Hoffken, 1994]. In contrast, when a hydrocarbon spacer of 3- to 11-carbons was appended between BBN(7-14) and the [16]aneS₄ macrocycle, the IC₅₀'s were found to be surprisingly relatively constant and in the 1-5 nM range. The following IC₅₀ values were obtained fro the unmetallated compounds BBN-22, -37, 46, and -47 (structures shown in FIG. 7).

| COMPOUND | IC₅₀ (nM) |
|---|---|
| BBN-22 | 3.01 ± 0.21 |
| BBN-37 | 1.79 ± 0.09 |
| BBN-46 | 2.34 ± 0.53 |
| BBN-47 | 4.19 ± 0.91 |

These data suggest that using relatively simple spacer groups to extend ligands some distance from the BBN binding region [e.g., BBN(8-14)] can produce derivatives that maintain binding affinities in the 1-5 nmolar range.

D. Cell Binding Studies With Metal Complexes:

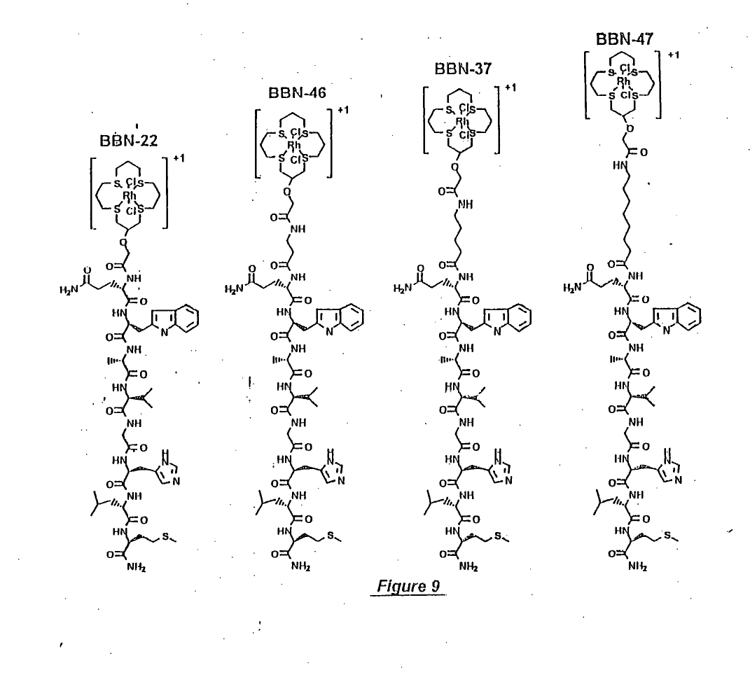

The following IC₅₀ values were obtained for the metallated Rhodium complexes shown on FIG. 9.

| COMPOUND | IC₅₀ (nM) |
|---|---|
| RhCl₂BBN-22 | 37.5 ± 10.5 |
| RhCl₂BBN-37 | 4.76 ± 0.79 |
| RhCl₂BBN-46 | 3.38 ± 0.69 |

The results illustrated in FIG. 9 show that when the RhCl₂-[16]aneS₄ complexes separated from Trp⁸ by only a glutamine (Glu⁷), the IC₅₀ of this conjugate (i.e., Rh-BBN-22) was 37.5 nM. However, when a five (5) carbon spacer or an eight (8) carbon spacer was present (i.e., Rh-BBN-37 and Rh-BBN-47), the IC₅₀'s remained below 5 nM. These data demonstrate that a straight chain spacer (along with glu⁷) to move the +1 charged Rh—S₄-chelate away from the BBN binding region will result in a metallated BBN analogue with sufficiently high binding affinities to GRP receptors for in vivo tumor targeting applications.

E. ¹⁰⁵Radiolabeled BBN Analogues:

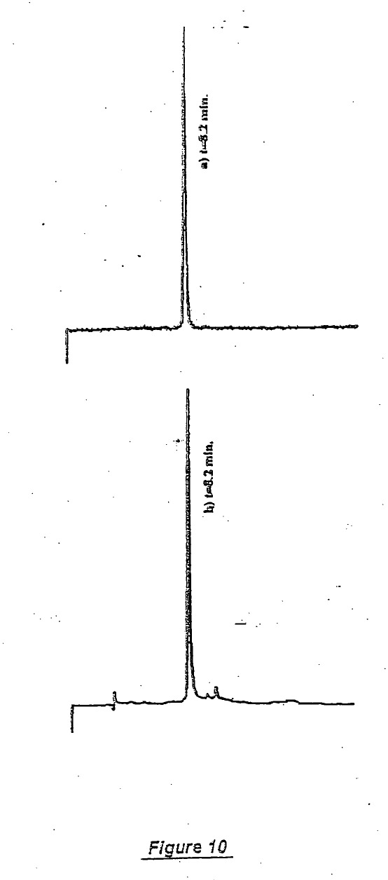

The ¹⁰⁵Rh conjugates of BBN-22, BBN-37, BBN46 and BBN47 were synthesized using a ¹⁰⁵Rh-chloride reagent from the Missouri University Research Reactor (MURR). This reagent was obtained as ¹⁰⁵Rh-chloride, a no-carrier-added (NCA) product, in 0.1-1M HCl. The pH of this reagent was adjusted to 4-5 using 0.1-1.0 M NaOH dropwise and it was added to approximately 0.1 mg of the [16]aneS₄-conjugated BBN derivatives in 0.9% aqueous NaCl and 10% ethanol. After the sample was heated at 80° C. for one hour, the ¹⁰⁵Rh-BBN analogues were purified using HPLC. In each case, a NCA or high specific activity product was obtained since the non-metallated S₄-BBN conjugates eluted at a retention time well after the ¹⁰⁵Rh-BBN conjugates eluted. For example, the retention time of ¹⁰⁵Rh-BBN-37 was 7.1 minutes while BBN-37 eluted at 10.5 minutes from a C-18-reversed phase column eluted with CH₃CN/H₂O containing 0.1% TFA as shown in FIG. 10A-B.

### Example 2

### Retention of ¹⁰⁵Rh-BBN Analogues in Cancer Cells

Once the radiometal has been specifically “delivered” to cancer cells (e.g., employing the BBN binding moiety that specifically targets GRP receptors on the cell surface), it is necessary that a large percentage of the “delivered” radioactive atoms remain associated with the cells for a period time of hours or longer to make an effective radiopharmaceutical for effectively treating cancer. One way to achieve this association is to internalize the radiolabeled BBN conjugates within the cancer cell after binding to cell surface GRP receptors.

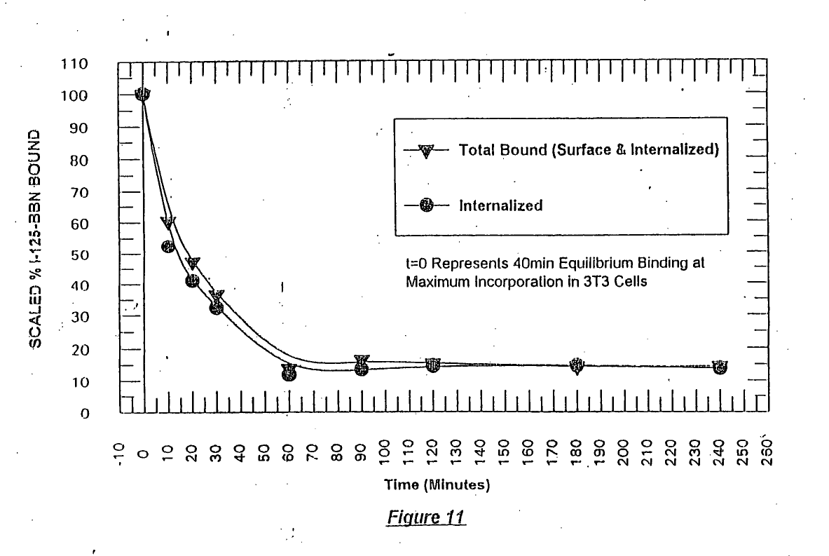

In the past, all of the work with synthetic-BBN analogues for treatment of cancers focused on synthesizing and evaluating antagonists [Davis et al., 1992; Hoffken, 1994; Moody et al., 1996; Coy et al., 1988; Cai et al., 1994; Moody et al., 1995; Leban et al., 1994; Cai et al., 1992]. After evaluating synthetic BBN analogues that would be predicted to be either agonists or antagonists, applicants found that derivatives of BBN(8-14) (i.e., those with the methionine or amidated methionine at BBN-14) are rapidly internalized (i.e., in less than two minutes) after binding to the cell surface GRP receptors. Several radiolabeled BBN(8-14) analogues that were studied to determine their internalization and intracellular trapping efficiencies were radioiodinated (i.e., ¹²⁵I) derivatives. The results of these studies demonstrated that despite rapid internalization after ¹²⁵I-labeled BBN analogue binding to GRP receptors in Swiss 3T3 cells, the ¹²⁵I was rapidly expelled from the cells [Hoffman et al., 1997] as shown in FIG. 11. Thus, these ¹²⁵I-BBN derivatives were not suitable for further development.

In contrast, the ¹⁰⁵Rh-BBN(8-14) derivatives that bind to GRP receptors are not only rapidly internalized, but there is a large percentage of the ¹⁰⁵Rh activity that remains trapped within the cells for hours (and in some cell lines>twenty four hours). This observation indicates that these radiometallated BBN derivatives have real utility as radiopharmaceuticals for in vivo targeting of neoplasms expressing GRP receptors.

Experiments designed to determine the fraction of a radiotracer internalized within cells were performed by adding excess ¹²⁵I- or ¹⁰⁵Rh-BBN derivatives to the cell incubation medium. After establishment of equilibrium after a forty minute incubation, the media surrounding the cells was removed and the cells were washed with fresh media containing no radioactivity. After washing, the quantity of radioactivity associated with the cells was determined (i.e., total counts per minute (TCPM) of ¹²⁵I or ¹⁰⁵Rh associated with the cells). The cells were then incubated in a 0.2M acetic acid solution (pH 2.5) which caused the surface proteins (incl., GRP receptors) to denature and release all surface bound radioactive materials. After removing this buffer and washing, the cells were counted again. The counts per minute (c.p.m.) associated with the cells at that point were only related to the ¹²⁵I or ¹⁰⁵Rh that remained trapped inside of the cells.

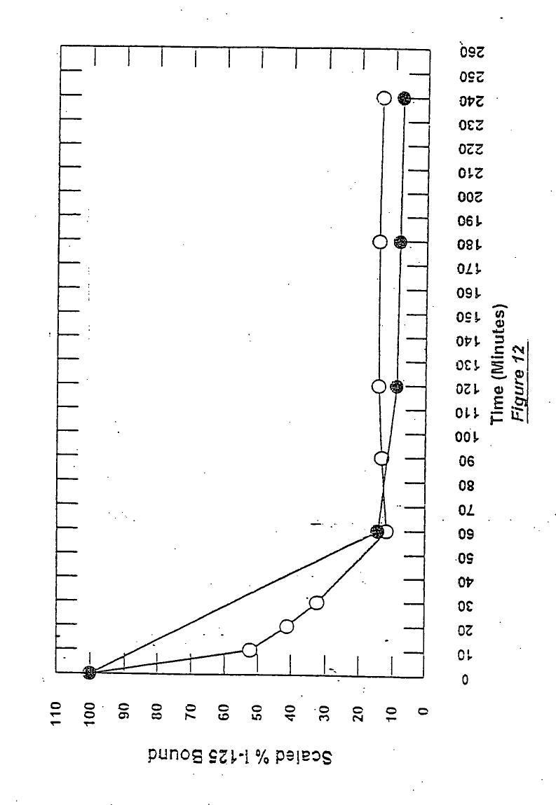

To determine intracellular retention, a similar method was employed. However, after washing the cells with fresh (non-radioactive) incubation media, the cells were incubated in the fresh media at different time periods after washing away all extracellular ¹²⁵I- or ¹⁰⁵Rh-BBN analogues. After each time period, the methods used to determine TOTAL c.p.m. and intracellular c.p.m. after washing with a 0.2M acetic acid solution at pH 2.5 were the same as described above and the percent ¹²⁵I or ¹⁰⁵Rh remaining trapped inside of the cells was calculated. FIG. 12 is a graph of results of efflux experiments using Swiss 3T3 cells with ¹²⁵I-Lys³-BBN. The results show that there is rapid efflux of the ¹²⁵I from inside of these cells with less than 50% retained at fifteen minutes and by sixty minutes, less than 20% remained as shown in FIG. 12.

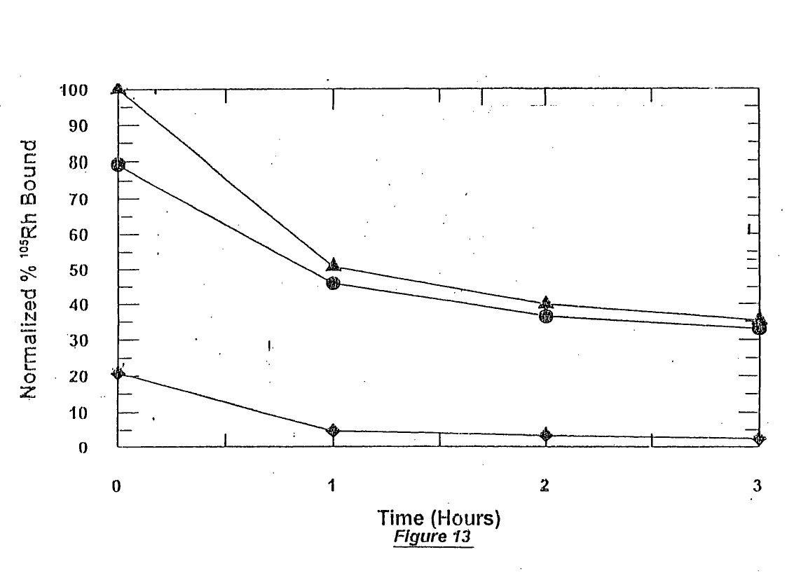

In contrast, studies with all of the ¹⁰⁵Rh-[16]aneS₄-BBN agonist derivatives that are internalized inside of the cells showed substantial intracellular retention of ¹⁰⁵Rh by the GRP receptor expressing cells. For example, results of studies using ¹⁰⁵Rh-BBN-37 (see FIG. 9) in conjunction with Swiss 3T3 cells showed that approximately 50% of the ¹⁰⁵Rh activity remains associated with the cells at sixty minutes post-washing and approximately 30% of ¹⁰⁵Rh remained inside of the cells after four hours as shown in FIG. 13. Note that at least 5% of the ¹⁰⁵Rh is surface bound at ≧sixty minutes.

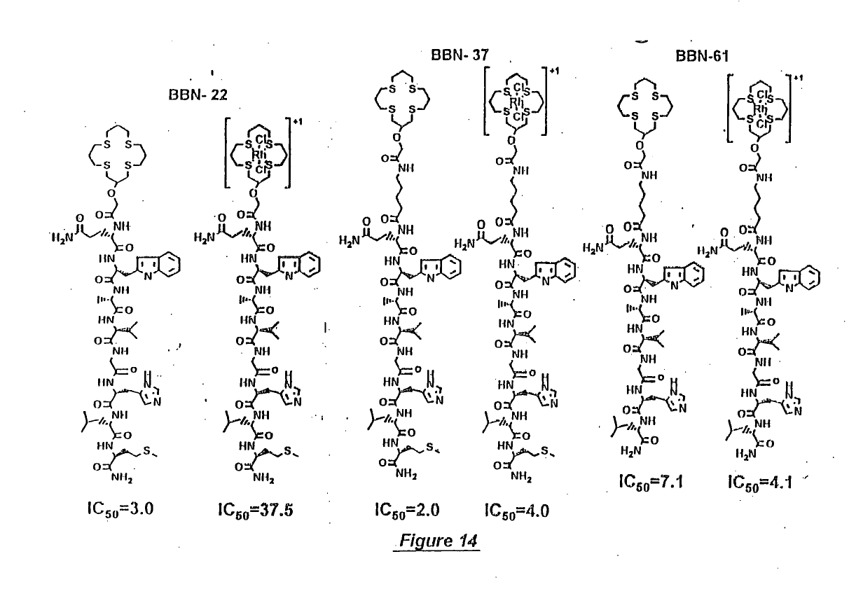

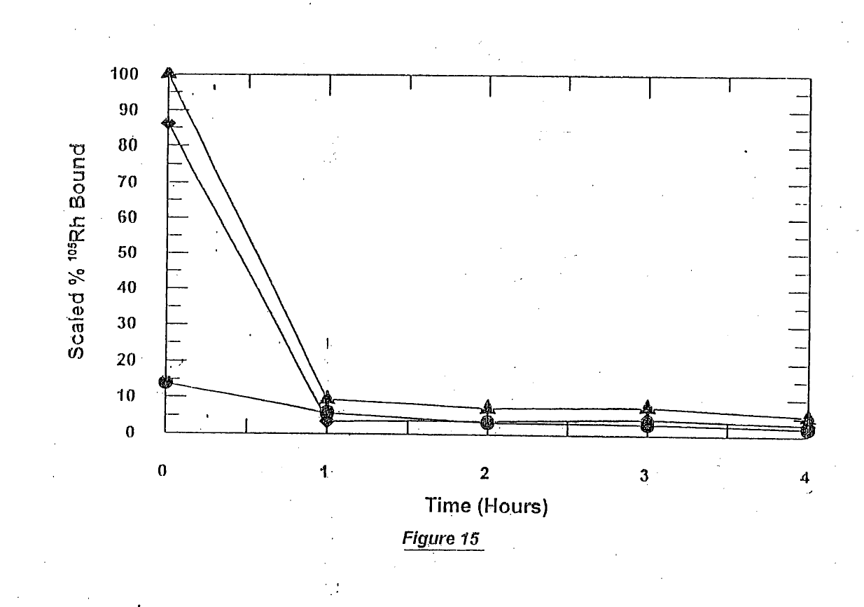

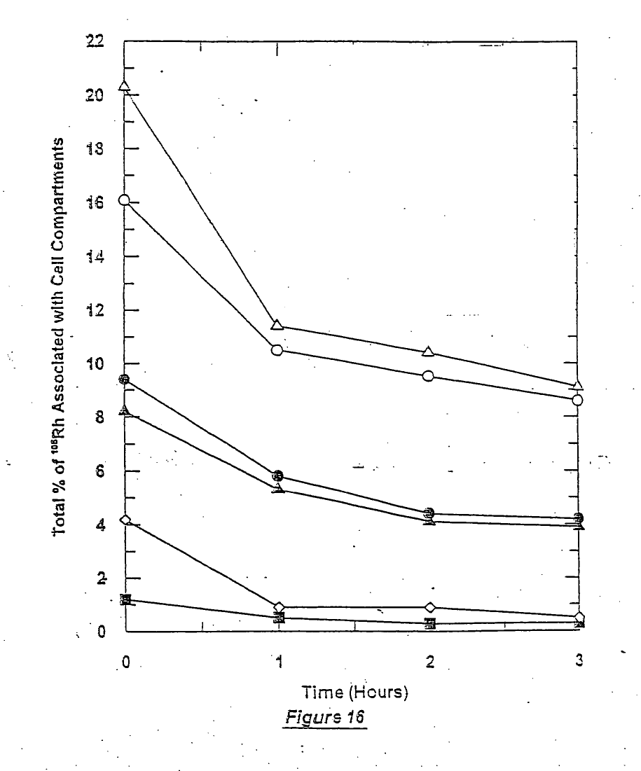

The ¹⁰⁵Rh-BBN derivatives shown in FIG. 9 all have an amidated methionine at position BBN-14 and are expected to be agonists [Jensen et al., 1993]. Therefore, they would be predicted to rapidly internalize after binding to GRP receptors on the cell surface [Reile et al., 1994; Bjisterbosch et al., 1995; Smythe et al., 1991], which was confirmed by applicants' data. Referring to FIG. 14, ¹⁰⁵Rh-BBN-61, a BBN analogue with no amino acid at position BBN-14 (i.e., a ¹⁰⁵Rh-BBN(8-13) derivative), was synthesized and studied. This BBN analogue has a high bonding affinity (i.e., IC₅₀=4.1 nM). This type of derivative is expected to be an antagonist and as such will not internalize [Jensen et al., 1993; Smythe et al., 1991]. Results of efflux studies with ¹⁰⁵Rh-BBN-61 using Swiss 3T3 cells showed that immediately following washing with fresh incubation buffer (i.e., t=0), essentially all of the ¹⁰⁵Rh associated with these cells is on the cell surface, as expected. Furthermore, after only one hour of incubation, less than 10% remained associated with these cells in any fashion (comparing the results with the antagonist (see FIG. 15) to those of the agonist (see FIG. 16)). These data indicate that ¹⁰⁵Rh-antagonists with structures similar to the ¹⁰⁵Rh-BBN agonists (i.e., those shown in FIG. 9) are not good candidates for development of radiopharmaceuticals since they are neither trapped in nor on the GRP receptor expressing cells to nearly the same extent as the radiometallated BBN agonists.

### Example 3

### Human Cancer Cell Line Studies

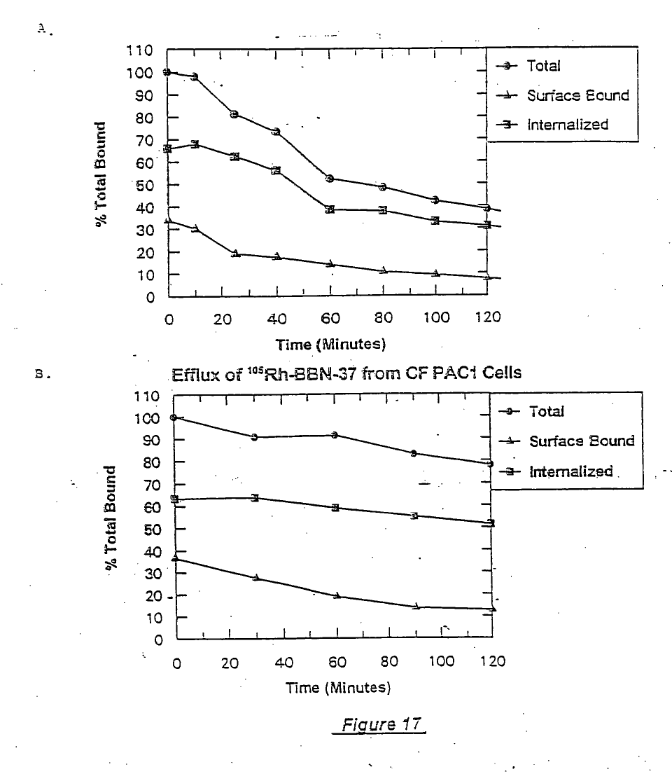

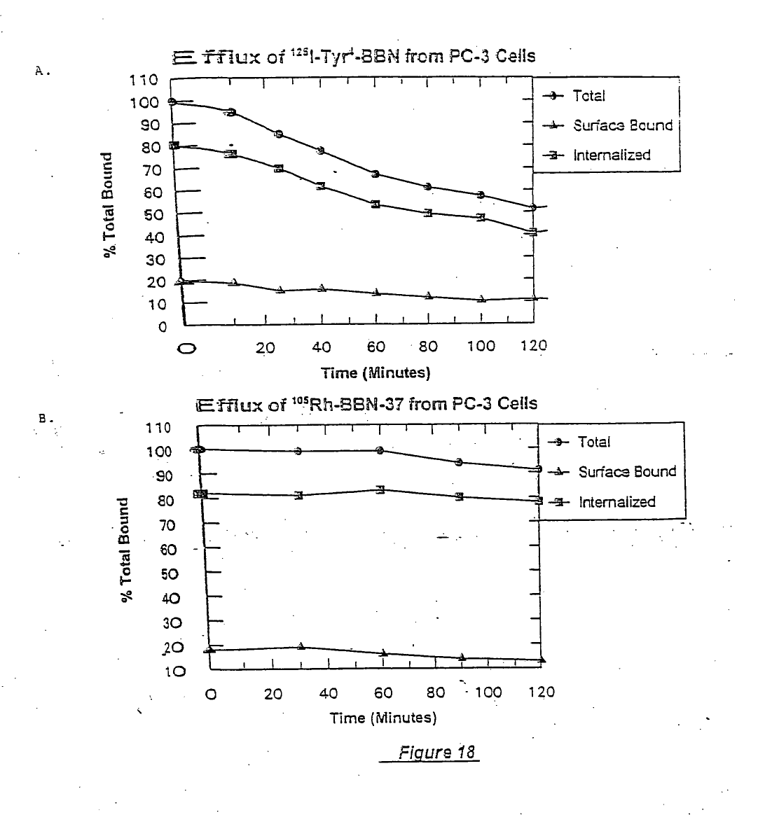

In vitro cell binding studies of ¹⁰⁵Rh-BBN-37 with two different human cancer cell lines that express GRP receptors (i.e., CF-PAC1 and PC-3 cell lines), which are tumor cells derived from patients with prostate CA and pancreatic CA, as shown in FIGS. 17A-B and 18A-B, respectively) were performed. Results of these studies demonstrated consistency with ¹⁰⁵Rh-BBN-37 binding and retention studies using Swiss 3T3 cells. Specifically, the binding affinity of Rh-BBN-37 was high (i.e., IC₅₀≅7 nM) with both human cancer cell lines as shown in Table 1. In addition, in all cells, the majority of the ¹⁰⁵Rh-BBN-37 was internalized and perhaps a major unexpected result was that the retention of the ¹⁰⁵Rh-tracer inside of the cells was significantly better than retention in Swiss 3T3 cells as shown in FIGS. 17 and 18. For example, it is particularly remarkable that the percentage of ¹⁰⁵Rh-BBN-37 that remained associated with both the CFPAC-1 and PC-3 cell line was >80% at two hours after removing the extracellular activity by washing with fresh incubation buffer (see FIGS. 17 and 18).

### Example 4

### In Vivo Studies

Biodistribution studies were performed by intravenous (I.V.) injection of either ¹⁰⁵Rh-BBN-22 or ¹⁰⁵Rh-BBN-37 into normal mice. In these studies, unanesthetized CF-1 mice (15-22 g, body wt.) were injected I.V. via the tail vein with between one (1) to five (5) uCi (37-185 KBq) of the ¹⁰⁵Rh-labeled agent. Organs, body fluids and tissues were excised from animals sacrificed at 30, 60 and 120 minutes post-injection (PI). The tissues were weighed, washed in saline (when appropriate) and counted in a NaI well counter. These data were then used to determine the percent injected dose (% ID) in an organ or fluid and the % ID per gram. The whole blood volume of each animal was estimated to be 6.5 percent of the body weight. Results of these studies are summarized in Tables 2 and 3.

Results from these studies showed that both the ¹⁰⁵Rh-BBN-22 and ¹⁰⁵Rh-BBN-37 were cleared from the blood stream, predominantly via the kidney into the urine. Specifically, 68.4±6.6% and 62.3±5.8% of the ID was found in urine at two hours PI of ¹⁰⁵Rh-BBN-22 and ¹⁰⁵Rh-BBN-37, respectively (see Tables 2 and 3). An unexpected finding was that the % ID of ¹⁰⁵Rh that remained deposited in the kidneys of these animals was only 2.4±0.6% ID and 4.6±1.3% ID at two hours PI of ¹⁰⁵Rh-BBN-22 and ¹⁰⁵Rh-BBN-37 (see Tables 2 and 3). This is much less than would be expected from previously reported data where radiometallated peptides and small proteins have exhibited renal retention of the radiometal that is >10% ID and usually much >10% [Duncan et al., 1997]. The reason for reduced renal retention of ¹⁰⁵Rh-BBN analogues is not known, however, this result demonstrates a substantial improvement over existing radiometallated peptides.

Biodistribution studies also demonstrated another important in vivo property of these radiometallated BBN analogues. Both ¹⁰⁵Rh-BBN-22 and ¹⁰⁵Rh-BBN-37 are efficiently cleared from organs and tissues that do not express GRP receptors (or those that do not have their GRP-receptors accessible to circulating blood). The biodistribution studies in mice demonstrated specific uptake of ¹⁰⁵Rh-BBN-22 and ¹⁰⁵Rh-BBN-37 in the pancreas while other non-excretory organs or tissues (i.e., heart, brain, lung, muscle, spleen) exhibited little or no uptake or retention (Tables 2 and 3). Both ¹⁰⁵Rh-BBN-22 and ¹⁰⁵Rh-BBN-37 were removed from the blood stream by both the liver and kidneys with a large fraction of the ¹⁰⁵Rh removed by these routes being excreted into the intestines and the bladder, respectively. It is important to note that the % ID/gm in the pancreas of ¹⁰⁵Rh-BBN-22 and ¹⁰⁵Rh-BBN-37 was 3.9±1.3% and 9.9±5.4%, respectively at 2 hour, PI. Thus, the ratios of % ID/gm of ¹⁰⁵Rh-BBN-22 in the pancreas relative to muscle and blood were 16.2 and 7.6, respectively. The ratios of % ID/gm of ¹⁰⁵Rh-BBN-37 in the pancreas relative to muscle and blood were 25.4 and 29.1, respectively. These data demonstrated selective in vivo targeting of these radiometallated BBN analogues to cells expressing GRP receptors [Zhu et al., 1991; Qin et al., 1994] and efficient clearance from non-target tissues. If cancer cells that express GRP receptors are present in the body, these results indicate radiometallated BBN analogues will be able to target them with a selectivity similar to the pancreatic cells.

A comparison of the pancreatic uptake and retention of ¹⁰⁵Rh-BBN-22 with ¹⁰⁵Rh-BBN-37 demonstrated that ¹⁰⁵Rh-BBN-37 deposits in the pancreas with a 2-fold better efficiency than ¹⁰⁵Rh-BBN-22 (i.e., 3.6±1.2% ID and 2.3±1.0% ID) for ¹⁰⁵Rh-BBN-37 at one and two hours PI, respectively, vs. 1.2±0.5% ID and 1.0±0.1% ID for ¹⁰⁵Rh-BBN-22 at one and two hours PI). This data is consistent with the >2-fold higher uptake and retention of ¹⁰⁵Rh-BBN-37 found in the in vitro studies shown in FIG. 16.

### Example 5

### Synthesis and In Vitro Binding Measurement of Synthetic BBN Conjugate Analogues Employing Amino Acid Chain Spacers

A. Synthesis

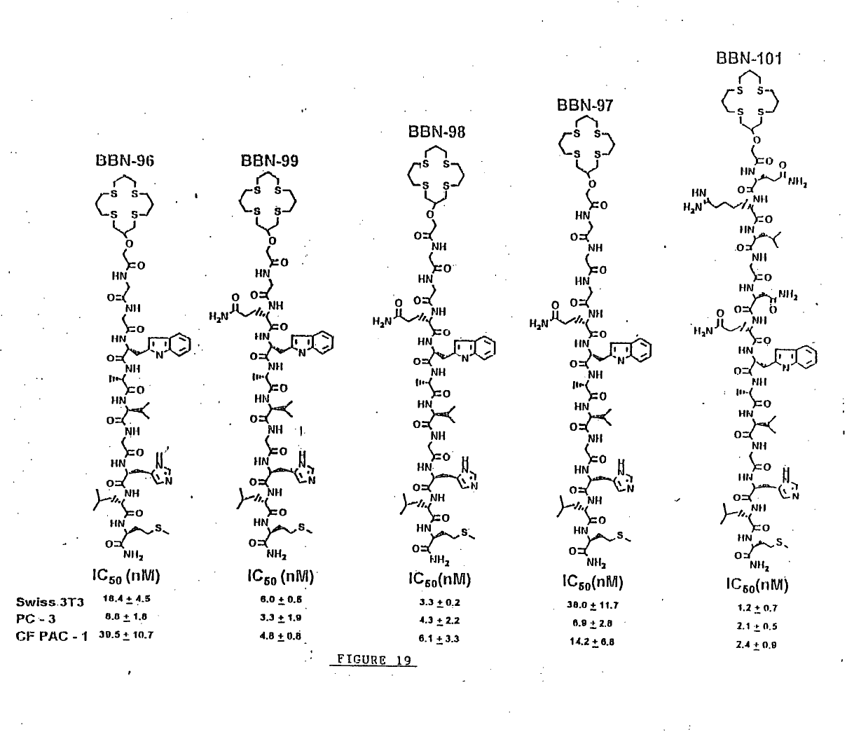

Five BBN analogues were synthesized by SPPS in which between 2 to 6 amino acid spacer groups were inserted to separate a S₄-macrocyclic chelator from the N-terminal trp⁸ on BBN(8-14) (FIG. 19). Each peptide was prepared by SPPS using an Applied Biosystems Model 432A peptide synthesizer. After cleavage of each BBN analogue from the resin using Trifluoracetic acid (TFA), the peptides were purified by C₁₈ reversed-phase HPLC using a Vydac HS54 column and CH₃CN/H₂O containing 0.1% TFA as the mobile phase. After collection of the fraction containing the desired BBN peptide, the solvent was evaporated. The identity of each BBN peptide was confirmed by FAB-mass spectrometry (Department of Chemistry—Washington University, St. Louis, Mo.).

Various amino acid sequences (in some cases containing different R group moieties) were conjugated to the N-terminal end of the BBN binding region (i.e., to BBN-8 or Trp⁸). BBN analogue numbers 96, 97, 98, 99 and 101 were synthesized as examples of N-terminal modified peptides in which the [16]aneS₄ macrocycle BFCA was separated from trp⁸ on BBN(8-14) by various amino acid sequences as shown in FIG. 19.

The [16]aneS₄ macrocyclic ligand was conjugated to selected tethered BBN analogues. The —OCH₂COOH group on the [16]and S₄ macrocycle derivative was activated via HOBt/HBTU so that it efficiently formed an amide bond with the terminal NH₂ group on the spacer side arm (following deprotection). The corresponding [16]aneS₄ tethered BBN derivatives were produced and examples of 5 of these derivatives (i.e., BBN-96, 97, 98, 99 and 101) are shown in FIG. 19. As previously described, each [16]aneS₄ BBN derivative was purified by reversed phase HPLC and characterized by FAB Mass Spectroscopy.

B. In Vitro Binding Affinities

The binding affinities of the synthetic BBN derivatives were assessed for GRP receptors on Swiss 3T3 cells, PC-3 cells and CF PAC-1 cells. The IC₅₀'s of each of derivative were determined relative to (i.e., in competition with) ¹²⁵I-Tyr⁴-BBN. The cell binding assay methods used to measure the IC₅₀'s is standard and was used by techniques previously reported [Jensen et al., 1993; Cai et al., 1992; Cai et al., 1994]. The methods used for determining IC₅₀'s with all BBN analogue binding to GRP receptors present on all three cell lines were similar. The specific method used to measure IC₅₀'s on Swiss 3T3 cells, is briefly described as follows:

Swiss 3T3 mouse fibroblasts are grown to confluence in 48 well microliter plates. An incubation media was prepared consisting of HEPES (11.916 g/l), NaCl (7.598 g/l), KCl (0.574 g/l), MgCl₂(1.106 g/l), EGTA (0.380 g/l), BSA (5.0 g/l), chymostatin (0.002 g/l), soybean trypsin inhibitor (0.200 g/l), and bacitracin (0.050 g/l). The growth media was removed, the cells were washed twice with incubation media, and incubation media was returned to the cells. ¹²⁵I-Tyr⁴-BBN (0.01 μCi) was added to each well in the presence of increasing concentrations of the appropriate competitive peptide. Typical concentrations of displacing peptide ranged from 10⁻¹² to 10⁻⁵ moles of displacing ligand per well. The cells were incubated at 37° C. for forty minutes in a 95% O₂/5% CO₂ humidified environment. At forty minutes post initiation of the incubation, the medium was discarded, and the cells were washed twice with cold incubation media. The cells were harvested from the wells following incubation in a trypsin/EDTA solution for five minutes at 37° C. Subsequently, the radioactivity, per well, was determined and the maximum % total uptake of the radiolabeled peptide was determined and normalized to 100%. A similar procedure was used in performing cell binding assays with both the PC-3 and CFₐ-PAC-1 human cancer cell lines.

C. Results of Binding Affinity Measurements

The IC₅₀ values measured for the BBN derivatives synthesized in accordance with this invention showed that appending a chelator via amino acid chain spacer groups via the N-terminal BBN-8 residue (i.e., Trp⁸) produced a variation of IC₅₀ values. For example, see IC₅₀ values shown for BBN 96, 97, 98 and 101 in FIG. 19. The observations are consistent with previous reports showing variable IC₅₀ values when derivatizing BBN(8-13) with a predominantly short chain of amino acid residues [Hoffken, 1994]. When the amino acid spacer groups used in BBN-98, 99 and 101 were appended between BBN(7-14) and the [16]aneS₄ macrocyle, the IC₅₀'s were found to be surprisingly constant and in the 1-6 nM range for all three cell lines (i.e., see IC₅₀ values shown in FIG. 19). These data suggest that using relatively simple spacer groups composed entirely of selected amino acid sequences to extend ligands some distance from the BBN region [e.g., BBN(8-14) can produce derivatives that maintain binding affinities in the 1-6 nmolar range.

D. Cell Binding Studies with Rh-BBN-Conjugates

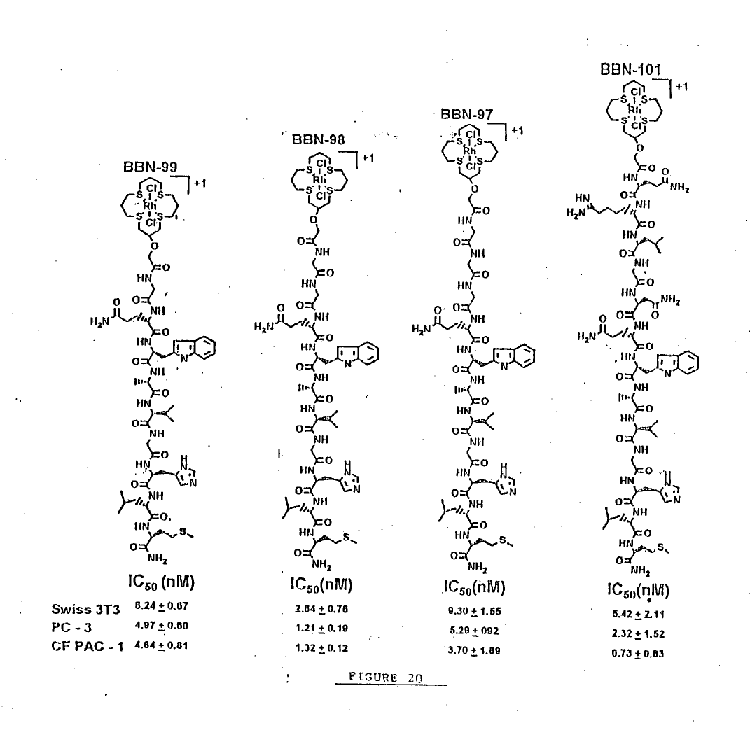

Results illustrated in FIG. 20 show that when the corresponding RhCl₂ [16]aneS₄ complex was separated from Trp⁸ on BBN(8-14) by the four different amino acid spacer groups (see FIG. 20), the IC₅₀'s of all four analogues (i.e., BBN-97, -98, -99, -101) were between 0.73 and 5.29 nmolar with GRP receptors on the PC-3 and CF PAC-1 cell lines. The IC₅₀'s for these same Rh-BBN conjugates were somewhat higher with the Swiss 3T3 cell line (FIG. 20). These data demonstrate that amino acid chain with spacer groups used to move the +1 charged Rh—S₄-chelate away from the BBN binding region will result in a metallated BBN analogue with sufficiently high binding affinities to GRP receptors for in vivo tumor targeting applications.

### Example 6

### Synthesis and In Vitro Binding Assessment of a ^{99m}Tc-Labeled Synthetic BBN Analogue

A. Synthesis

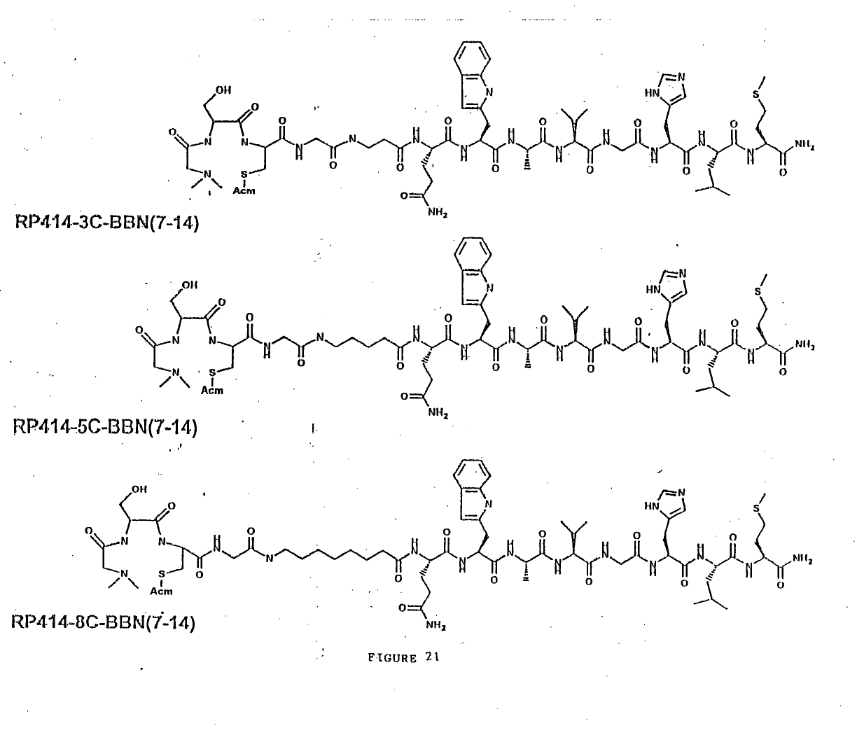

Several tetradentate chelating frameworks have been used to form stable 99 mTc or 188Re labeled peptide and protein conjugates [Eckelman, 1995; Li et al., 1996b; Parker, 1990; Lister-James et al., 1997]. Many of these ligand systems contain at least one thiol (—SH) donor group to maximize rates of formation and stability (both in vitro and in vivo) of the resultant Tc(V) or Re(V) complexes [Parker, 1990; Eckelman, 1995]. Results from a recent report indicates that the bifunctional chelating agent (BFCA) (dimethylglycyl-L-seryl-L-cyteinyl-glycinamide (N3S-BFCA) is capable of forming a well-defined complex with ReO+3 and TcO+3 [Wong et al., 1997]. Since this ligand framework can be synthesized by SPPS techniques, this N3S-BFCA was selected for use in forming of Tc-99m-BBN-analogue conjugates. Three different N3S-BFCA conjugates of BBN(7-14) were synthesized (BBN-120, -121 and -122) as shown in FIG. 21 by SPPS. BBN-120, BBN-121 and BBN-122 represent a series of analogues where the N3S-BFCA is separated from the BBN(7-14) sequence by a 3, 5 and 8 carbon spacer groups (FIG. 21). Each peptide was synthesized and purified using the SPPS and chromatographic procedures outlined in Example 1. The thiol group on cysteine was protected using the ACM group, which is not cleaved during cleavage of these BBN-conjugates from the resin using TFA. The identity of BBN-120, -121 and -122 was confirmed by FAB mass spectrometry. Synthesis and purification of the N3S-BFCA could also be readily accomplished using SPPS methods, followed by HPLC purification (see Example 1). The ACM group was used to protect the thiol group on cysteine during synthesis and cleavage from the resin.

B. In Vitro Binding Affinities

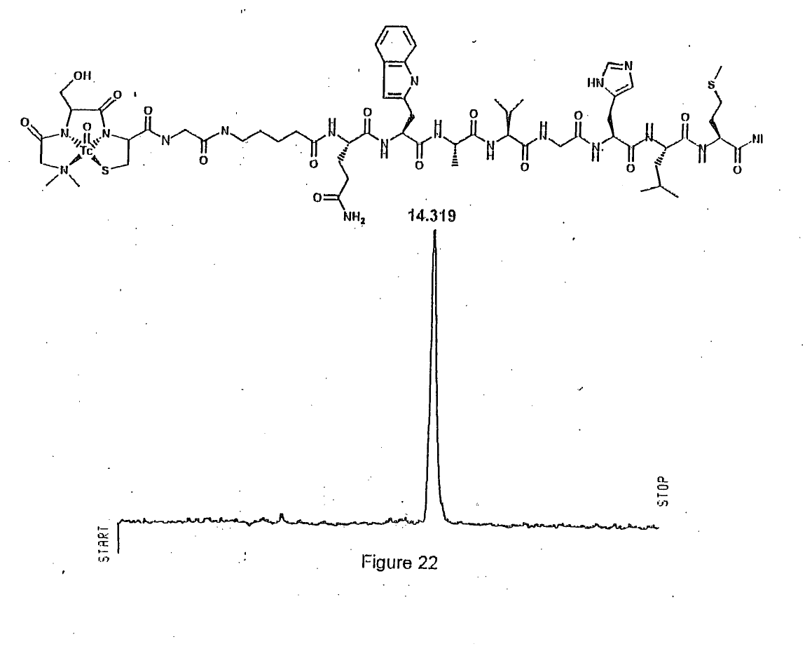

Synthesis of 99 mTc-BBN-122 (FIG. 22) was prepared by two methods [i.e., (1) by transchelation of 99 mTcO+3 from 99 mTc-gluconate or (2) by formation of the “preformed” 99 mTc-BFCA complex followed by —COOH activation with tetrafluorophenyl and subsequent reaction with the C5-carbon spacer group appended to BBN(7-14)]. In both cases, the 99 mTc-labeled peptide formed is shown in FIG. 22. The structure of this Tc-BBN-122 conjugate was determined by using non-radioactive Re(the chemical congener of Tc). In these studies, the “preformed” ReO+3 complex with the N3S-BFCA was prepared by reduction of ReO4; with SnCl2 in the presence of excess N3S-BFCA dissolved in sodium phosphate buffered water at pH 6-6.5 by a method previously published [Wong et al., 1997]. After purification of the ReO-N3S-BFCA complex, the structure of this chelate was shown (by Mass-Spect) to be identical to that previously reported [Wong et al., 1997].

The ReO-N-3-S-BFCA complex was converted to the activated trifluorophenyl (TFP) ester by adding 10 mg of the complex to 6 mg (dry) EDC and the 50 μl of TFP. After the solution was vortexed for one minute, CH3CN was added until disappearance of cloudiness. The solution was incubated for one hour at RT and purified by reversed-phase HPLC. To prepare the ReO-N3S-BFCA complex BBN-122 conjugate (FIG. 22), one μl of the HPLC fraction containing the ReO-N3S-BFCA complex was added to a solution containing 1 mg of the C8-tethered BBN(7-14) peptide in 0.2 N NaHCO3 at pH 9.0. After incubation of this solution for one hour at RT, the sample was analyzed and purified by reversed-phase HPLC. The yield of Re-BBN-122 was approximately 30-35%.

The method for preparation of the corresponding 99 mTc-BBN-122 conjugate, using the “preformed” 99 mTcO-N3S-BFCA complex, was the same as described above with the “preformed” ReO-N3S-BFCA complex. In this case, 99 mTc04, from a 99Mo/99 mTc generator, was reduced with an aqueous saturated stannous tartrate solution in the presence of excess N3S-BFCA. The yields of the 99 mTc-BBN-122 product using this “preformed” method were approximately 30-40%. Reversed phase HPLC analysis of the 99 mTc-BBN-122, using the same gradient elution program¹ as used for analysis of the Re-BBN-122 conjugate, showed that both the 99 mTc-BBN-122 and 188Re-BBN-122 had the same retention time (i.e., 14.2-14.4 min) (See FIG. 22). This provides strong evidence that the structure of both the 99 mTc-BBN-122 and Re-BBN-122 are identical.

The binding affinities of BBN-122 and Re-BBN-122 were assessed for GRP receptors on Swiss 3T3 cells, PC-3 cells and CFPAC-1 cells that express GRP receptors. The IC50's of each derivative were determined relative to (i.e., in competition with) ¹²⁵I-Tyr4-BBN (the Kd for ¹²⁵I-Tyr4-BBN for GRP receptors in Swiss 3T3 cells is reported to be 1.6±0.4 nM) [Zhu et al., 1991]. The cell binding assay methods used to measure the IC₅₀'s is standard and was used by techniques previously reported [Leban et al., 1994; Cai et al., 1994; Cai et al., 1992]. The methods used for determining IC₅₀'s with all GRP receptor binding of GRP receptors on all cell lines was similar and has been described previously for the other BBN-analogues and Rh-BBN analogues described in this document.

C. Results of Binding Affinity Measurements
¹Gradient elution program used in these studies was as follows.

Flow 1.5 ml/minute

Solvent A=HO with 0.1% TFA

Solvent B=CHCN with 0.1% TFA

| Time (minutes) | % A/% B |
|---|---|
| 0 | 95/5 |
| 25 | 30/70 |
| 35 | 95/5 |

The IC50 values measured for BBN-122 and Re-BBN-122 synthesized in accordance with this invention showed that appending an 8-carbon hydrocarbon chain spacer linked to the N3S1-BFCA and the corresponding Re complex (i.e., Trp8) produced BBN conjugates with IC50 values in a 1-5 nmolar range (See Table A). When 99 mTc-BBN-122 was incubated with these same cells, it was shown that ≧nmolar concentrations of BBN displaced this 99 mTc conjugate by >90%. This result demonstrates that 99 mTc-BBN-122 has high and specific binding affinity for GRP receptors. These data suggest that using relatively simple spacer groups to extend the N3S ligand framework and the corresponding Tc- or Re-N3S1, complexes some distance from the BBN binding region can produce derivatives that maintain binding affinities in the 1-5 nmolar range. TABLE A.

TABLE A
Summary of IC50 values for GRP receptor binding for the
non-metallated BBN-122 conjugate or the Re-BBN-112
conjugate in two cell lines (PC-3 and CF-PAC-1 cell lines
that express GRP receptors). The IC50 values were
measured using cell binding assays relative to 1251-Tyr4-BBN.

IC50 (nmolar)

| Conjugate | PC-3 | CF-PAC1 |
|---|---|---|
| BBN-122 | 3.59 ± 0.75 (n = 6) | 5.58 ± 1.92 (n = 14) |
| Re-BBN-122 | 1.23 ± 0.56 (n = 12) | 1.47 ± 0.11 (n = 6) |

### Example 7

### Retention of 99 mTc-BBN-122 in Human Cancer Cells PC-3 and CF-PAC-1 cells)

Once the radiometal has been specifically “delivered” to cancer cells (e.g., employing the BBN binding moiety that specifically targets GRP receptors on the cell surface), it is necessary that a large percentage of the “delivered” radioactive atoms remain associated with the cells for a period time of hours or longer to make an effective radiopharmaceutical for effectively treating cancer. One way to achieve this association is to internalize the radiolabeled BBN conjugates within the cancer cell after binding to cell surface GRP receptors.

Experiments designed to determine the fraction 99 mTc-BBN-122 internalized within cells were performed by the same method previously described for 105Rh-BBN-37. Briefly, excess 99 mTc-BBN-122 was added to PC-3 or CFPAC-1 cell incubation media and allowed to establish equilibrium after a forty minute incubation. The media surrounding the cells was removed and the cells were washed with fresh media containing no radioactivity. After washing, the quantity of radioactivity associated with the cells was determined (i.e., total counts per minute 99 mTc associated with cells). The PC-3 and CFPAC-1 cells were then incubated in a 0.2M acetic acid solution (pH2.5) which caused the surface proteins (including GRP receptors) to denature and release all surface bound radioactive materials. After removing this buffer and washing, the cells were counted again. The counts per minute (c.p.m.) associated with the cells at that point were only related to the 99 mTc that remained trapped inside of the PC-3 or CFPAC-1 cells.

To determine intracellular retention of 99 mTc activity, a similar method was employed. However, after washing the cells with fresh (non-radioactive) incubation media, the cells were incubated in the fresh media at different time period after washing away all extracellular 99 mTc-BBN-122. After each time interval, the methods used to determine total c.p.m. and intracellular c.p.m. by washing with a 0.2M acetic acid solution at pH 2.5.

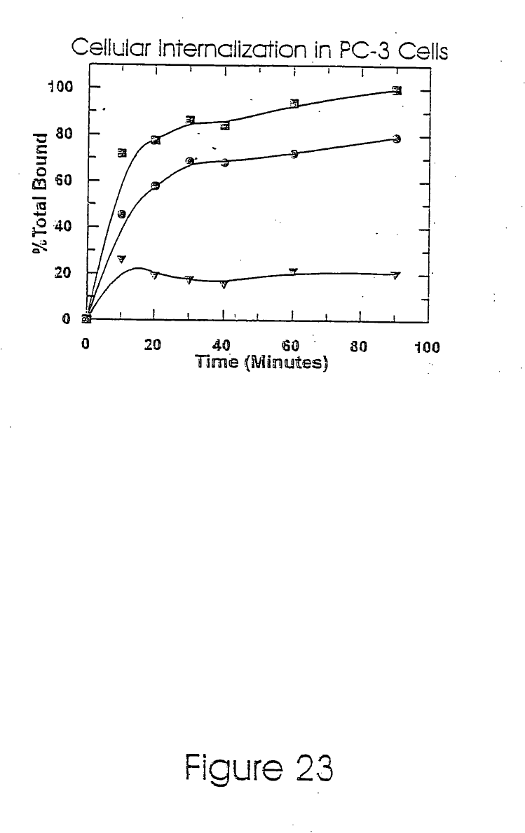

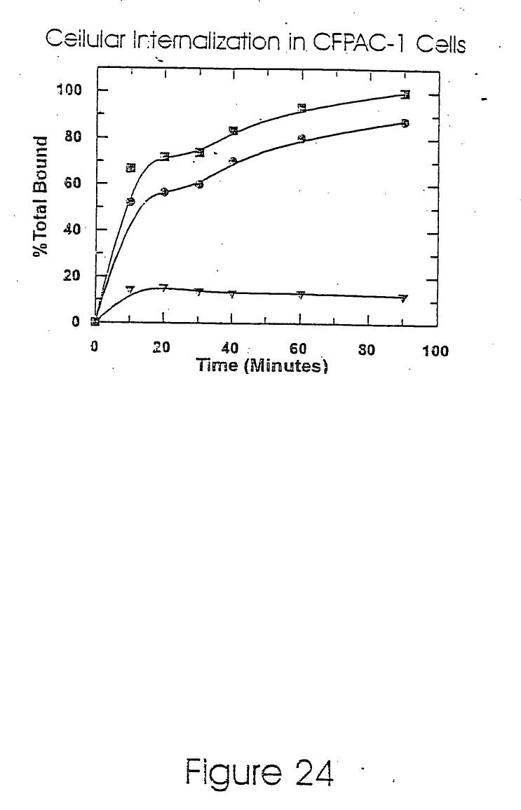

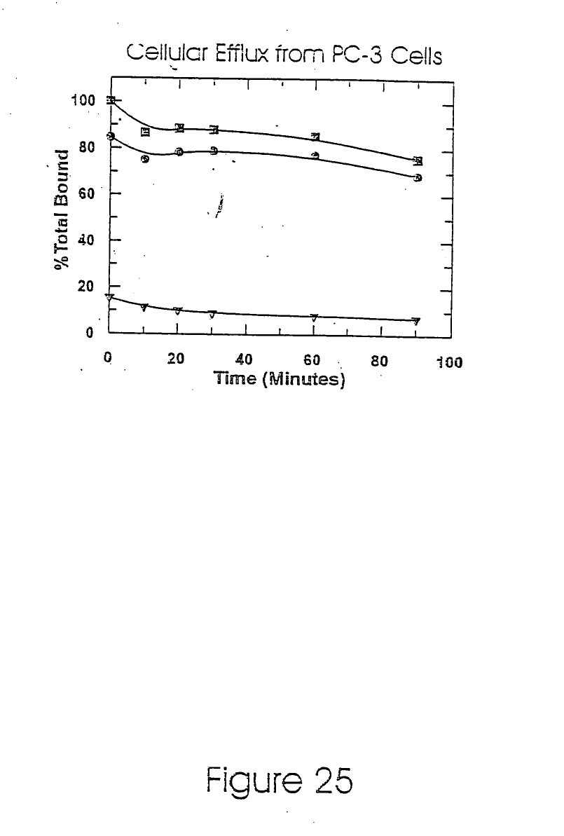

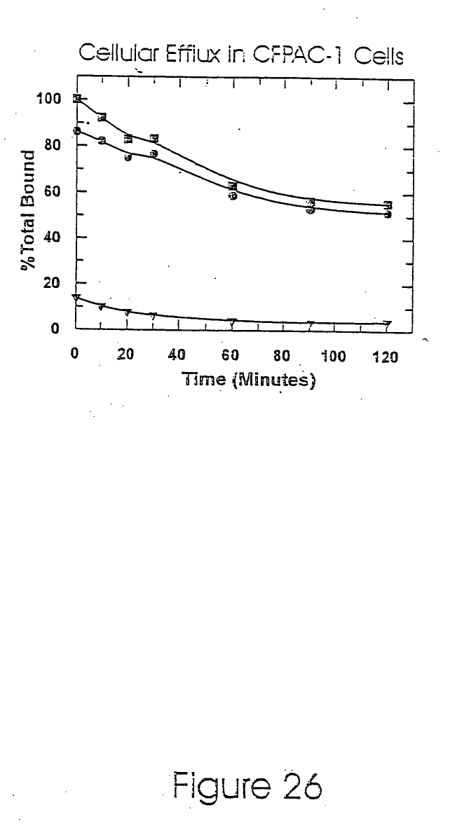

Studies with the 99 mTc-BBN-122 agonist show that it is internalized inside of the PC-3 and CFPAC-1 cells (FIGS. 23-26) and that substantial intracellular retention of 99 mTc by the GRP receptor expressing cells occurs. For example, results of studies using 99 mTc-BBN-122 in conjunction with PC-3 cells showed a high rate of internalization (FIG. 23) and that approximately 75% of the 99 mTc activity remains associated with the cells at ninety minutes post-washing (FIG. 25). Almost all of this 99 mTc cell-associated activity is inside of the PC-3 cells. Similar results were also found with the CFPAC 1 cells where there is also a high rate of 99 mTc-BBN-122 internalization (FIG. 24) and relatively slow efflux of 99 mTc from the cells (i.e., 50-60% retention at 120 minutes post-washing (FIG. 26).

The 99 mTc-BBN-122 peptide conjugate shown in FIG. 22 has an amidated methionine at position BBN-14 and is expected to be an agonist [Jensen et al., 1993]. Therefore, it would be predicted to rapidly internalize after binding to GRP receptors on the cell surface [Bjisterbosch et al., 1995; Smythe et al., 1991], which is confirmed by applicants' data in FIG. 23-26.

### Example 8

### In Vivo Studies

Biodistribution studies were performed by intravenous (I.V.) injection of 99 mTc-BBN-122 into normal mice. In these studies, unanesthetized CF-1 mice (15-22 g, body wt.) were injected I.V. via the tail vein with between one (1) to five (5) μCi (37-185 KBq) of 99 mTc-BBN-122. Organs, body fluids and tissues were excised from animals sacrificed at 0.5, 1, 4 and 24 hours post-injection (PI). The tissues were weighed, washed in saline (when appropriate) and counted in a NaI well counter. These data were then used to determine the percent injected dose (% ID) in an organ or fluid and the % ID per gram. The whole blood volume of each animal was estimated to be 6.5 percent of the body weight. Results of these studies are summarized in Tables B and C.

Results from these studies showed that 99 mTc-BBN-122 is cleared from the blood stream predominantly via the hepatobiliary pathway showing about 35% of the 99 mTc-activity cleared via the kidney into the urine. Specifically, 33.79±1.76% of the ID was found in urine at one hour PI (Table B). The retention of 99 mTc activity in the kidneys and liver is very low (Table B). This is much less than would be expected from previously reported data where radiometallated peptides and small proteins have exhibited renal retention of the radiometal that is >10% ID and usually much >10% [Duncan et al., 1997]. The reason for reduced renal retention of 99 mTc-BBN-122 is not known, however, this result demonstrates a substantial improvement over existing radiometallated peptides.

Biodistribution studies also demonstrated another important in vivo property of 99 mTc-BBN-122 in that it is efficiently cleared from organs and tissues that do not express GRP receptors (or those that do not have their GRP-receptors accessible to circulating blood). The biodistribution studies in mice demonstrated specific uptake of 99 mTc-BBN-122 in the pancreas while other non-excretory organs or tissues (i.e., heart, brain, lung, muscle, spleen) exhibited little or no uptake or retention. 99 mTc-BBN-122 is removed from the blood stream by both the liver and kidneys with a large fraction of the 99 mTc removed by these routes being excreted into the intestines and the bladder, respectively. It is important to note that the % ID/gm in the pancreas of 99 mTc-BBN-122 is 12.63%/gm at 1 hour and drops to only 5.05% at the 4 hour PI (Table C). Thus, the ratios of % ID/gm of 99 mTc-BBN-122 in the pancreas relative to muscle and blood were 92.2 and 14.78 at 4 hour PI, respectively. These data demonstrated selective in vivo targeting of this 99 mTc-labeled BBN analogue to cells expressing GRP receptors [Zhu et al., 1991; Qin et al., 1994] and efficient clearance from non-target tissues. If cancer cells that express GRP receptors are present in the body, these results indicate 99 mTc-BBN analogues will be able to target them with a selectivity similar to the pancreatic cells.

### Example 9

### Materials and Methods For Examples 9 and 10

The following abbreviations are used in the examples and derived from the following amino acids:
ava=5-amino valeric acid
aoc=8-amino octanoic acid
aun=11-amino undecanoic acid

Reagents and Apparatus. All chemicals were obtained from either Aldrich Chemicals (St. Louis, Mo.) or Fisher Scientific (Pittsburgh, Pa.). All chemicals and solvents used in these studies were reagent grade and used without further purification. The Resin and fmoc-protected amino acids were purchased from Calbiochem-Novabiochern Corp (San Diego, Calif.) and the other peptide reagents from Applied Biosystems, Inc (Foster City, Calif.). The DOTA-tris(t-butyl ester) was purchased from Macrocyclics (Dallas, Tex.) and the fmoc-protected w-amino alkyl carboxylic acids from Advanced ChemTech (Louisville, Ky.). ¹²⁵I-Tyr4-Bombesin (¹²⁵I-Tyr4-BBN) was obtained from NEN Life Sciences Products, Inc (Boston, Mass.). 111InC13 was obtained from Mallinckrodt Medical, Inc (St. Louis, Mo.) as a 0.05N HCl solution. 90Y was obtained from Perkin-Elmer (Biclerica, Mass.) as an HCl solution. Electrospray mass spectral analyses were performed by Synpep Corporation and T47D (Dublin, Calif.). Human prostate cancer PC-3 cells and MDA-MB-231 breast cancer cells were obtained from American Tissue Culture Collection (ATCC) and maintained and grown in the University of Missouri Cell and Immunology Core facilities. CF-1 mice were purchased from Charles River Laboratories (Wilmington, Mass.) and maintained in an in house animal facility.

Solid Phase Peptide Synthesis (SPPS). Peptide synthesis was carried out on a Perkin Elmer—Applied Biosystems Model 432 automated peptide synthesizer employing traditional fmoc chemistry with HBTU activation of carboxyl groups on the reactant with the N-terminal amino group on the growing peptide anchored via the C-terminus to the resin. Rink Amide MBHA resin (25 μmol), fmoc-protected amino acids with appropriate side-chain protections (7 μmol), fmoc-protected amino alkyl carboxylic acids (75 μmol) and DOTA-tris(t-butyl ester) (75 gmol) were used for the synthesis. The final products were cleaved by a standard procedure using a cocktail containing thioanisol, water, ethanedithiol and trifluoroacetic acid in a ratio of 2:1:1:36 and precipitated into methyl-t-butyl ether. Typical yields of the crude peptides were 80-85%. Crude peptides were purified by HPLC and the solvents were removed on a SpeedVac concentrator. The purified peptides were characterized by electrospray mass spectrometry. The mass spectral analysis results are shown in Table 4.

High performance liquid chromatography (HPLC). High performance liquid chromatography (HPLC) analyses for DOTA conjugates were performed on a Waters 600E system equipped with Varian 2550 variable absorption detector, Packard Radiometic 1 5OTR flow scintillation analyzer, sodium iodide crystal radiometric detector, Eppendorf TC-50 column temperature controller and Hewlett Packard HP3395 integrators. A Phenomenex Jupiter C-18 (5 μm, 300 A0, 4.6×250 mm) column was used with a flow rate of 1.5 ml/minute HPLC solvents consisted of H₂O containing 0.1% trifluoroacetic acid (Solvent A) and acetonitrile containing 0.1% trifluoroacetic acid (Solvent B). HPLC gradient conditions for 0 spacer to 8 carbon spacer analogs begin with a solvent composition of 80% A and 20% B followed by a linear gradient to 70% A:30% B over 30 minutes. HPLC gradient conditions for the 11 carbon spacer analysis are solvent composition of 75% A and 25% B followed by a linear gradient to 50% A:50% B over 30 minutes.

Indium metallation. A solution of the unmetallated DOTA-BBN conjugates as shown in table 4 (5.0 mg) in 0.2M tetramethylammonium acetate (0.5 ml) was added to indium trichloride (InCl3) (10.0 mg). The pH of the reaction mixture was adjusted to 5.5 (Scheme 1). The reaction mixture was incubated for 1 hour at 80° C. The resultant In-DOTA-BBN conjugate (Scheme 1) was purified by reversed-phase HPLC and analyzed by electrospray mass spectrometry. The mass spectral analysis results are shown in Table 4. The pure product was obtained as a white powder with a typical yield of 50-60%.

111Indium/90Yttrium labeling. An aliquot of 111InCl3 (1.0 mCi, 50 μl) was added to a solution of the unmetallated DOTA-BBN (100 μg) conjugates shown in Table 4 in 0.2M tetramethylammonium acetate (500 μl). The pH of the reaction mixture was adjusted to 5.6. The reaction mixture was incubated for 1 hour at 80° C. An aliquot of 0.002M EDTA (50 μl) was added to the reaction mixture to complex the unreacted 111In+3. The resultant 111In-DOTA-BBN conjugate was obtained as a single product and purified by reversed-phase HPLC. The purified 111In-DOTA-BBN conjugate was then concentrated by passing through a 3M Empore C-18 HD high performance extraction disk (7 mm/3 ml) cartridge and eluting with 33% ethanol in 0.1M NaH2PO4 buffer (400 μl). The concentrated fraction was then diluted with 0.1M NaH2PO4 buffer (2.3 ml, pH-7) to make the final concentration of ethanol in the solution <5%. The 90Y DOTA-BBN complex was similarly prepared.

In Vitro Cell Binding Studies. The IC50 of the various In-DOTA-BBN conjugates was determined by a competitive displacement cell binding assay using 125I-Tyr4-BBN. Briefly 3×104 cells suspended in RPMI medium 1640 at pH-7.4 containing 4.8 mg/ml HEPES, 0.1 μg/ml Bacitracin and 2 mg/ml BSA, were incubated at 37° C. and a 5% CO₂ atmosphere for 40 minutes in the presence of 20,000 cpm ¹²⁵I-Tyr4-BBN and increasing concentration of the In-DOTA-BBN conjugates. After the incubation, the reaction medium was aspirated and cells were washed four times with media. The radioactivity bound to the cells was counted in a Packard Riastar gamma counting system. The % ¹²⁵I-Tyr4-BBN bound to cells was plotted vs. increasing concentrations of In-DOTA-BBN conjugates to determine the respective IC50 values.

Internalization and efflux studies. In vitro studies to determine the degree of internalization of the 111In-DOTA-8-Aoc-BBN[7-14]NH2 conjugate were carried out by a method similar to that of described by Rogers, et al. These studies were performed by incubating 3×104 cells suspended in RPMI medium 1640 at pH-7.4 containing 4.8 mg/ml HEPES, 0.1 μg/ml Bacitracin and 2 mg/ml BSA, at 37° C. and a 5% CO₂ atmosphere for 40 minutes in presence of 20,000 cpm 111In-DOTA-8-Aoc-BBN[7-14]NH2 conjugate. After the incubation, the reaction medium was aspirated and cells were washed with media. The percent of cell-associated activity as a function of time (in the incubating medium at 37° C.) was determined. The percentage radioactivity trapped in the cells was determined after removing activity bound to the surface of the cells by washing with a pH-2.5 (0.2M acetic acid and 0.5M NaCl) buffer 1, 2, 3 and 4 hours afterwards.

In vivo pharmacokinetic studies in CF-i mice. The biodistribution and uptake of 111In-DOTA-BBN conjugates in CF-1 mice was studied. The mice (average weight, 25 g) were injected with aliquots (50-100 μl) of the labeled peptide solution (55-75 kBq) in each animal via the tail vein. Tissues and organs were excised from the animals sacrificed at 1 hour post-injection. The activity counted in a NaI counter and the percent injected dose per organ and the percent injected dose per gram were calculated. The percent injected dose (% ID) in the blood was estimated assuming a blood volume equal to 6.5% of the total body weight. Receptor blocking studies were also carried out where excess (100 μg) BBN was administered to animals along with the 111In-DOTA-8-Aoc-BBN[7-14]NH2.

In vivo pharmacokinetic studies in human tumor bearing SCID mice. The biodistribution studies of the 111In and 90Y conjugates were determined in SCID mice bearing human tumor xenografts of either PC-3 (human androgen independent prostate cancer cell origin) or MDA-MB-231 (human breast cancer cell origin) cell lines. The xenograft models were produced by bilateral flank inoculation of 5×106 cells (PC-3 or MDA-MB-231 cells) per site. Four to six weeks post inoculation, palpable tumors were observed. At this point, the mice were injected with 4 μCi of the complex in 100/μL of isotonic saline via the tail vein. The mice were euthanized and tissues and organs were excised from the animals at selected times post-injection (p.i.), including 15 minutes, 30 minutes, 1 hour, 4 hours, 24 hours, 48 hours, and 72 hours p.i. Subsequently, the tissues and organs were weighed and counted in a NaI well counter and the percent injected dose (% ID) and % ID/g of each organ or tissue calculated. The % ID in whole blood was estimated assuming a whole-blood volume of 6.5% the total body weight.

In vivo pre-clinical evaluation of single dose radiotherapy in human tumor bearing SCID mice. Preclinical therapeutic evaluation of the 90Y-DOTA-8-Aoc-BBN[7-14]NH2 conjugate was performed in SCID mice bearing PC-3 human androgen independent prostate cancer cell human tumor xenografts. The xenograft model was produced by bilateral flank inoculation of 5×106 PC-3 cells per site. Twenty one days post inoculation when palpable tumors appeared, single dose administration of 90Y-DOTA-8-Aoc-BBN[7-14]NH2 was initiated. Baseline weights, hematology profiles, and tumor measurements were obtained immediately prior to therapy administration. Four groups of animals were utilized; a saline placebo, a 5 mCi/kg single dose, a 10 mCi/kg single dose, and a 20 mCi/kg single dose. Tumor measurements and weights were obtained twice weekly throughout the 14 weeks post injection.

Discussion

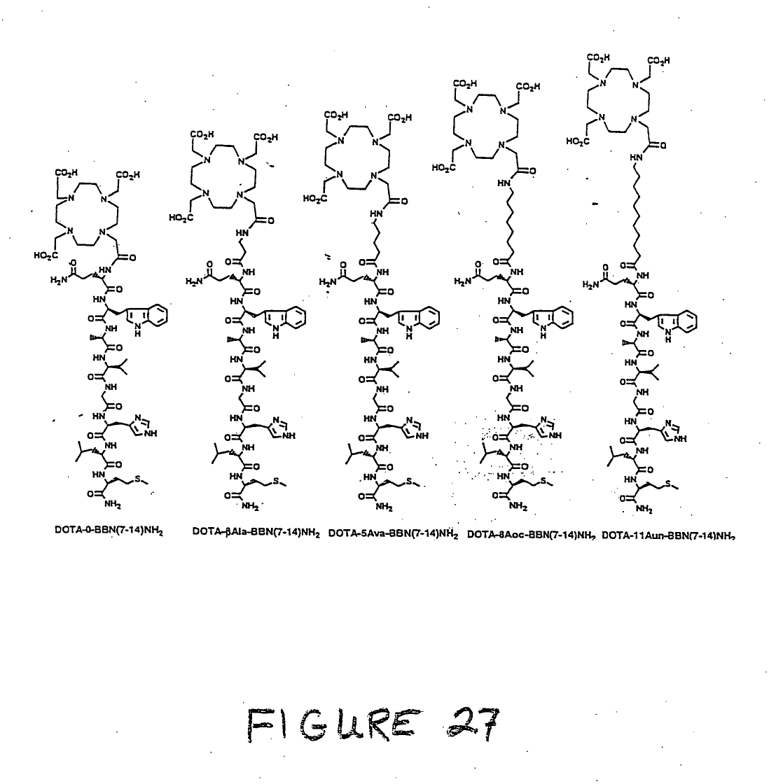

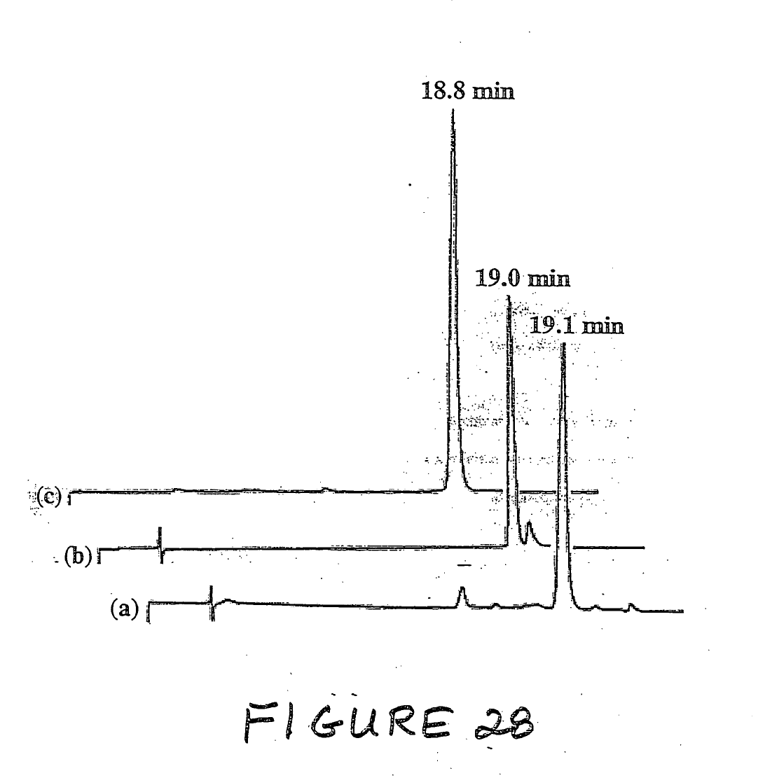

A series of BBN-agonists containing the DOTA chelation system separated by spacers have been synthesized and characterized. [FIGS. 27 and 28; Tables 4 and 20]

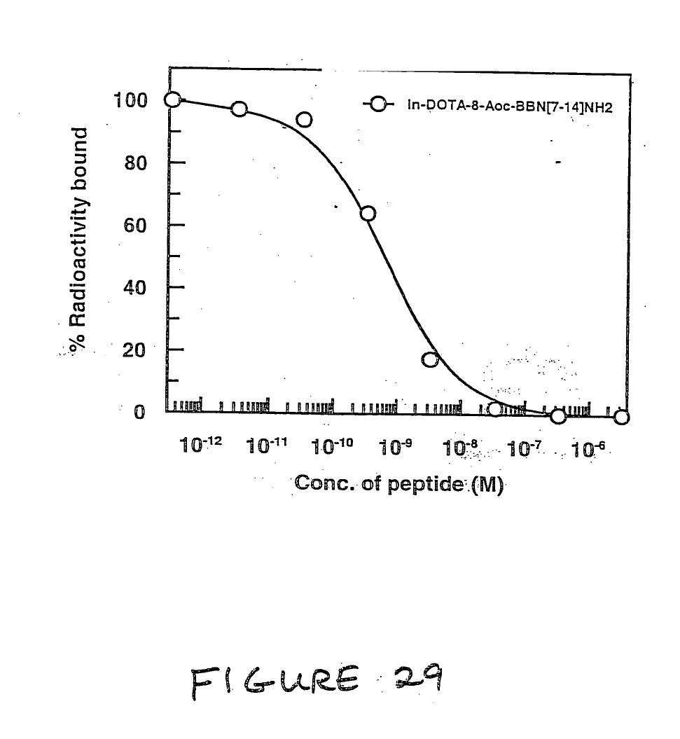

The in vitro binding affinity of the Indium-BBN analogs was measured in two cell lines, the human prostate cancer cell line, PC-3, and the human breast cancer cell line, T47D. Of the compounds tested, optimum binding of the In-DOTA-8-Aoc-BBN[7-14]NH2 analog was demonstrated in both cell lines examined. [FIG. 29 & Table 5]

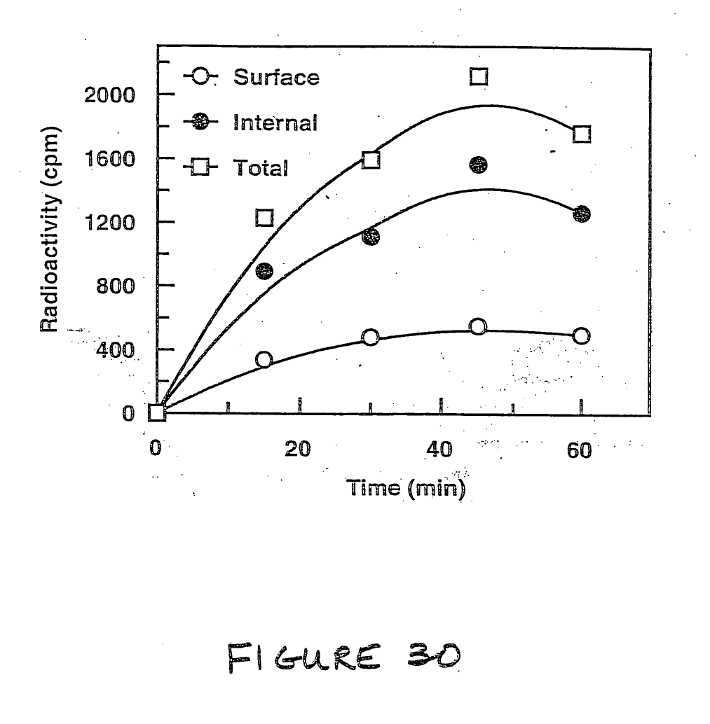

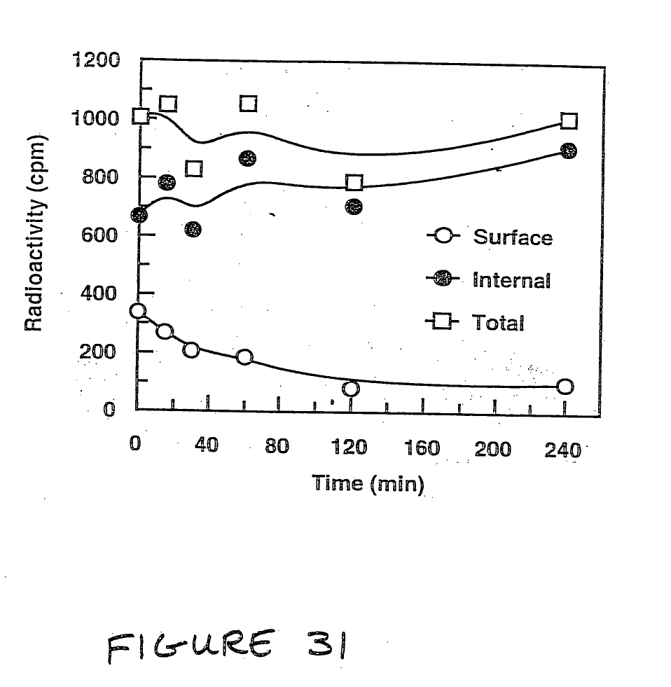

The In-DOTA-8-Aoc-BBN[7-14]NH2 analog underwent rapid receptor mediated endocytosis using an in vitro PC-3 cell assay system. Once internalized within PC-3 cells, the In-DOTA-8-Aoc-BBN[7-14]NH2 analog remained retained within the cells for a prolonged time period. [FIGS. 30 and 31]

In vivo analysis of the DOTA-BBN analogs in CF1 mice demonstrates that 111In-DOTA-5-Ava-BBN[7-14]NH2, 111In-DOTA-8-Aoc-BBN[7-14]NH2, 111In-DOTA-I 1-Aun-BBN[7-14]NH2 all target GRP receptor expression in vivo based on high uptake and accumulation of these compounds in the normal pancreas. Increasing hydrocarbon spacer length linking the DOTA metal chelation moiety to the GRP receptor binding moiety (BBN[7-14]NH2) results in compounds with increased hydrophobicity which subsequently shifts the clearance of these agents from the renal system (hydrophilic agents) to the hepatobiliary system (hydrophobic agents). [Tables 6 and 7] Specific in vivo GRP receptor binding was demonstrated by performing competitive blocking assays in CF1 normal mice, where >98% of the normal receptor mediated uptake of 111In-DOTA-8-Aoc-BBN[7-14]NH2 in normal pancreatic tissue was blocked by co-administration of an excess of bombesin. [Tables 8 and 9]

Prolonged PC-3 human prostate tumor uptake was demonstrated for 111In-DOTA-8-Aoc-BBN[7-14]NH2 and 90Y-DOTA-8-Aoc-BBN[7-14]NH2 using a xenograft mouse model. [Tables 10-13]

Prolonged MDA-MB-23 1 human breast tumor uptake was demonstrated for 111In-DOTA-8-Aoc-BBN[7-14]NH2 using a xenograft mouse model. [Table 14]

These in vivo pharmacokinetic studies in CF1 mice have demonstrated that the radiometallated bombesin analogues (111In and 90Y) clear from the blood pool, into the renal-urinary excretion pathway.

Competitive Binding Assay Results

The 111In and 90Y complexes of one lead candidate, DOTA-8-Aoc-BBN[7-14]NH2, have been synthesized and evaluated in vitro and in vivo. In vitro competitive binding assays, employing PC-3 human prostate tumor cells, demonstrated an average IC50 value of 1.69 nM for the In-DOTA-8-Aoc-BBN[7-14]NH2 complex.

In Vivo Pharmacokinetic Results in PC-3 Tumor Bearing Mice

In vivo pharmacokinetic studies of 111In-DOTA-8-Aoc-BBN[7-14]NH2 in PC-3 prostate tumor bearing mice conducted at 1, 4, 24, 48, and 72 hours p.i. revealed efficient clearance from the blood pool (0.92±0.58% ID, 1 hour p.i.) with excretion through the renal and hepatobiliary pathways (87% ID and 8.5% ID, at 24 hours p.i., respectively). Similar pharmacokinetic properties were observed with 90Y-DOTA-8-Aoc-BBN[7-14]NH2. Tumor targeting of PC-3 xenografted SCID mice resulted in tumor uptake and retention values of 3.63±1.11% ID/g, 1.78±1.09% ID/g, and 1.56±0.45% ID/g obtained at 1, 4, and 24 hours p.i. respectively, for the 111In-DOTA-8-Aoc-BBN-[7-1 4]NH2 complex. 90Y-DOTA-8-Aoc-BBN[7-1 4]NH2 exhibited nearly identical PC-3 tumor uptake and retention values of 2.95±0.99% ID/g, 1.98±0.66% ID/g, and 1.08±0.37% ID/g at 1, 4, and 24 hr p.i., respectively. Initial therapeutic assessment of the 90Y complex in PC-3 xenografted mice demonstrated that radiation doses of up to 20 mCi/kg were well tolerated with overall survival exhibiting a dose dependent response.

These pre-clinical observations show that peptide conjugates of this type exhibit properties suitable as clinical therapeutic/diagnostic pharmaceuticals.,

### Example 10

### Binding of DOTA-BBN Conjugates in Human Breast Cancer Cell Lines

Expression of Gastrin Releasing Peptide receptors (GRP-Rs) in a variety of cancers including breast, prostate, small cell lung, and pancreatic is well known. Recently, the first positive clinical images of GRP-R expression in human metastatic breast cancer patients were obtained [C. Van de Wiele et al., Eur. J. Nucl. Med. (2000) 27:1694-1699] with the compound, 99 mTc-N3S-5-Ava-BBN(7-14)NH2, initially developed in our laboratory. The continued efforts in the development of GRP targeted radiopharmaceuticals has led to the synthesis of a series of DOTA incorporated peptides for the complexation of 111In/90Y.

Methods: Six synthetic peptides were constructed in an X—Y-Z fashion where X=the DOTA chelation system, Y=the linking arm, and Z=the BBN(7-14)NH2 sequence. The six peptides differed in the selection of linking arms, comprising either amino acid tethers; -G-G-G-, -G-S-G-, or S-G-S—, or alkyl carbon chain tethers; 5-Ava, 8-Aoc, or 11-Aun. The In complexes of all peptides were prepared, purified by RPHPLC, and characterized by ES-MS as described in example 9.

Results: Pharmacokinetic studies conducted in CF1 mice revealed that 111In-DOTA-8-Aoc-BBN(7-14)NH2 exhibited optimum clearance kinetics while maintaining selective and high in vivo GRP receptor targeting. 111In-DOTA-8-Aoc-BBN(7-14)NH2 exhibited an IC50 value of 1.23±0.25 nM for the GRP receptor expressed by the T47D human breast cancer cell line. Pharmacokinetic studies of 111In-DOTA-8-Aoc-BBN(7-14)NH2 conducted in MDA-MB-231 human breast cancer cell line xenografted SCID mice demonstrated specific tumor targeting with 0.83±0.23% ID/g obtained at 1 hour post injection. Residualization of the radiolabel within the tumor was observed with 46%, and 28%, of the initial uptake retained at 4, and 24 hours, respectively.

Conclusion: These results show that GRP-R specific radiopharmaceuticals incorporating the DOTA chelation system are beneficial for the development of diagnostic/therapeutic matched pair agents to target breast cancer.

### Example 11

### Lutetium DOTA-BBN Compounds

A conjugate, 177Lu-DOTA-8-Aoc-BBN[7-14]NH2, was routinely prepared in high yield (≧95%) by addition of 177LuCl3 to an aqueous solution (Ammonium Acetate) of DOTA-8-Aoc-BBN[7-14]NH2 (3.4×10-8 mols) [pH=5.5, Temp.=800C, RT=1 hour]. RCP determination demonstrated the stability of the conjugate over a wide range of pH values over a time course of 24 hours. The HPLC chromatogram of 177Lu-DOTA-8-Aoc-BBN[7-14]NH2 showed a retention time of 19.0 minutes. Under identical chromatographic conditions, DOTA-8-Aoc-BBN[7-14]NH2 has a retention time of 20.5 minutes, allowing for peak purification of the radiolabeled conjugate. Collection of and counting of the 177Lu-DOTA-8-Aoc-BBN[7-14]NH2 eluant peak in a NaI well counter further demonstrated the stability of the new complex as >95% of the activity loaded onto the column was recovered as a singular species.

The biodistribution studies of 177Lu-DOTA-8-Aoc-BBN[7-14]NH2 were determined in tumor bearing (PC-3), SCID mice (TABLE 19). This 177Lu-conjugate cleared efficiently from the bloodstream within 1 hour post-injection. For example, 0.62±0.44% ID remained in whole blood at 1 hour p.i. The majority of the activity was excreted via the renal-urinary excretion pathway (i.e., 67.41±2.45% at 1 hour p.i. and 85.9±1.4% at 24 hour p.i.), with, the remainder of the radioactivity being excreted through the hepatobiliary pathway. Receptor-mediated, tumor targeting of the PC-3 xenografted SCID mice resulted in tumor uptake and retention values of 4.22±1.09% ID/g, 3.03±0.91% ID/g, and 1.54±1.14% ID/g at 1, 4, and 24 hours, respectively.

### Experimental

To 50 μl (3.4×10⁻⁸ mols) of DOTA-8-Aoc-BBN[7-14]NH2 in 50 μL of 0.2M Ammonium Acetate was added 150 μL of 0.4M Ammonium Acetate. To this solution was added 50 μL of 177LuCl3 (2 mCi in 0.05N HCl, Missouri University Research Reactor). The solution was allowed to incubate at 800C for 1 hour, after which 50 kg of 0.002M EDTA was added in order to scavenge uncomplexed Lutetium. Quality control of the final product was determined by reversed-phase HPLC. Peak purification of the labeled species was performed by collecting the sample from the HPLC eluant, into a solution of 1 mg/mL bovine serum albumin/0.1M Na2HPO4. All further analyses were carried out using the HPLC-purified products.

HPLC analysis of each of the new compounds was performed using an analytical C-18 reversed phase column (Phenomenex, 250×4.6 mm, 5 μm). The mobile phase consisted of a linear gradient system, with solvent A corresponding to 100% water with 0.1% trifluoroacetic acid and solvent B corresponding to 100% acetonitrile with 0.1% trifluoroacetic acid. The mobile phase started with solvent compositions of 80% A:20% B. At time=30 minutes, the solvent compositions were 70% A:30% B. Solvent compositions of the mobile phase remained as such (70% A:30% B) for a period of two minutes before being changed to 100% B. At time=34 minutes, the solvent composition was again changed to 80% A:20% B for column re-equilibration. The flow rate of the mobile phase was 1.5 mL/min. The chart speed of the integrator was 0.5 cm/min. The results of these analyses are shown in Table 20.

In vivo analysis of the DOTA-BBN analogs in CF1 mice demonstrates that 149 Pm-DOTA-5-Ava-BBN[7-14]NH2 and 149 Pm-DOTA-8-Aoc-BBN[7-14]NH2 target GRP receptor expression in vivo based on high uptake and accumulation of these compounds in the normal pancreas, which contain high levels of the GRP receptor [Tables 15 and 16]

In vivo analysis of the DOTA-BBN analogs in CF1 mice demonstrates that 177Lu-DOTA-5-Ava-BBN[7-1 4]NH2, 177Lu-DOTA-8-Aoc-BBN[7-1 4]NH2, and 177Lu-DOTA-11-Aun-BBN[7-14]NH2 all target GRP receptor expression in vivo based on high uptake and accumulation of these compounds in the normal pancreas. [Tables 17 and 18]

The biodistribution studies of 177Lu-DOTA-8-Aoc-BBN[7-14]NH2 were determined in SCID mice bearing human prostate cancer, PC-3 tumors. The mice were injected with 4 μCi of the complex in 100 μL of isotonic saline via the tail vein. The mice were euthanized by cervical dislocation. Tissues and organs were excised from the animals following at 1 hour, 4 hour, and 24 hours post-injection (p.i.). Subsequently, the tissues and organs were weighed and counted in a NaI well counter and the percent injected dose (% ID) and % ID/g of each organ or tissue calculated. The % ID in whole blood was estimated assuming a whole-blood volume of 6.5% the total body weight.

Prolonged PC-3 human prostate tumor uptake was demonstrated for 177Lu-DOTA-8-Aoc-BBN[7-14] using a xenograft mouse model of human prostate cancer. [Table 19 and 20]

### CONCLUSION

This pre-clinical evaluation of 177Lu-DOTA-8-Aoc-BBN[7-14]NH2 and 149 Pm-DOTA-8-Aoc-BBN[7-14]NH2 suggests the potential for peptide conjugates of this type to be used as site-directed, therapeutic radiopharmaceuticals.

TABLE B
Biodistribution of 99mTc-BBN-122 in normal CF-1 mice
at 0.5, 1, 4 and 24 hr post-IV injection. Results
expressed as % ID/organ

| | % Injected Dose/Organ^{a} | | | |
|---|---|---|---|---|
| Organ^{c} | 30 min | 1 hr | 4 hr | 24 hr |
| Blood^{d} | 3.52 ± 2.16 | 1.08 ± 0.34 | 0.59 ± 0.24 | 0.12 ± 0.01 |
| Liver | 4.53 ± 0.93 | 4.77 ± 1.40 | 1.49 ± 0.32 | 0.32 ± 0.06 |
| Stomach | 2.31 ± 0.45 | 1.61 ± 0.81 | 1.75 ± 0.20 | 0.30 ± 0.06 |
| Lg. Intestine^{b} | 2.84 ± 0.32 | 24.17 ± 7.91 | 23.85 ± 7.02 | 0.61 ± 0.14 |
| Sm. Intestine^{b} | 43.87 ± 1.51 | 23.91 ± 9.08 | 5.87 ± 7.09 | 0.42 ± 0.06 |
| Kidneys^{b} | 1.49 ± 0.19 | 1.15 ± 0.10 | 0.55 ± 0.06 | 0.20 ± 0.01 |
| Urine^{b} | 26.78 ± 1.05 | 33.79 ± 1.76 | ˜35 | ˜35 |
| Muscle | 0.02 ± 0.01 | 0.01 ± 0.00 | 0.01 ± 0.01 | 0.01 ± 0.01 |
| Pancreas | 5.30 ± 0.63 | 3.20 ± 0.83 | 1.21 ± 0.13 | 0.42 ± 0.17 |

^{a}Each value in the table represents the mean and SD from 5 animals in each group
^{b}At 4 and 24 hr, feces containing 99Tc had been excreted from each animal and the % ID in the urine was estimated to be approximately 60% of the ID.
^{c}All other organs excised (incl. Brain, heart, lung and spleen) shown <0.10% at t ≧ 1 hr.
^{d}% ID in the blood estimated assuming the whole blood volume is 6:5% of the body weight.

TABLE C
Biodistribution of 99mTc-BBN-122 in normal CF-1 mice
at 0.5, 1, 4 and 24 hr post I.V. injection. Results expressed
as % ID/gm.

| | % Injected Dose/gma | | | |
|---|---|---|---|---|
| Organ | 30 min | 1 hr | 4 hr | 24 hr |
| Blood^{b} | 2.00 ± 1.28 | 0.63 ± 0.19 | 0.34 ± 0.11 | 0.08 ± 0.00 |
| Liver | 2.70 ± 0.41 | 3.14 ± 0.81 | 0.96 ± 0.20 | 0.22 ± 0.05 |
| Kidneys | 3.99 ± 0.76 | 3.10 ± 0.31 | 1.58 ± 0.15 | 0.64 ± 0.07 |
| Muscle | 0.23 ± 0.08 | 0.13 ± 0.02 | 0.05 ± 0.01 | 0.01 ± 0.01 |
| Pancreas | 16.89 ± 0.95 | 12.63 ± 1.87 | 5.05 ± 0.42 | 1.79 ± 0.71 |

P/B1 and P/M Update Ratios

| | | | | |
|---|---|---|---|---|
| Pancreas/ | 8.42 | 19.76 | 14.78 | 20.99 |
| Blood | | | | |
| Pancreas/ | 73.16 | 93.42 | 92.25 | 142.76 |
| Muscle | | | | |

aEach value in the table represents the mean and SD from 5 animals in each group.
^{b}% ID in the blood estimated assuming the whole blood volume is 6:5% of the body weight.

TABLE D
Biodistribution of ^{99m}Tc-BBN-122 in PC-3 tumor
bearing SCID mice at 1, 4 and 24 hr post-I.V. injection.
Results expressed as % ID/organ.

| Tumor Line: | | | |
|---|---|---|---|
| PC-3 | % ID per Organ^{a} | | |
| Organ^{c} | 1 hr | 4 hr | 24 hr |
| Blood^{b} | 1.16 ± 0.27 | 0.47 ± 0.06 | 0.26 ± 0.05 |
| Liver | 1.74 ± 0.64 | 0.72 ± 0.10 | 0.29 ± 0.05 |
| Stomach | 0.43 ± 0.18 | 0.29 ± 0.22 | 0.08 ± 0.02 |
| Lg. Intestine | 9.18 ± 19.42 | 42.55 ± 8.74 | 0.64 ± 0.17 |
| Sm. Intestine | 46.55 ± 16.16 | 2.13 ± 0.76 | 0.31 ± 0.04 |
| Kidneys | 1.16 ± 0.20 | 0.60 ± 0.06 | 0.16 ± 0.01 |
| Urine^{d} | 32.05 ± 12.78 | ˜35 | ˜35 |
| Muscle | 0.01 ± 0.00 | 0.00 ± 0.00 | 0.00 ± 0.00 |
| Pancreas | 1.69 ± 0.61 | 1.05 ± 0.13 | 0.34 ± 0.08 |
| Tumor | 1.00 ± 0.78 | 0.49 ± 0.08 | 0.49 ± 0.25 |

^{a}Each value in the table represents the mean and SD from 5 animals in each group.
^{b}At 4 and 24 hr, feces containing ^{99m}Tc had been excreted from each animal and the % ID in the urine was estimated to be approximately 60% of the ID.
^{c}All other organs excised (incl. brain, heart, lung and spleen) showed <0.10% at t ≧ 1 hr.
^{d}% ID in the blood estimated assuming the whole blood volume is 6:5% of the body weight.

TABLE E
Biodistribution of ^{99m}Tc-BBN-122 in PC-3 tumor
bearing SCID mice at 1, 4 and 24 hr post-I.V. injection.
Results expressed as % ID/Gm.

| Tumor Line: | | | |
|---|---|---|---|
| PC-3 | % ID per gm^{a} | | |
| Organ | 1 hr | 4 hr | 24 hr |
| Blood^{b} | 0.97 ± 0.26 | 0.31 ± 0.03 | 0.18 ± 0.04 |
| Liver | 2.07 ± 0.88 | 0.64 ± 0.05 | 0.26 ± 0.04 |
| Kidneys | 4.80 ± 1.33 | 2.23 ± 0.35 | 0.60 ± 0.04 |
| Muscle | 0.18 ± 0.12 | 0.06 ± 0.03 | 0.05 ± 0.04 |
| Pancreas | 10.34 ± 3.38 | 5.08 ± 1.12 | 1.47 ± 0.23 |
| Tumor | 2.07 ± 0.50 | 1.75 ± 0.61 | 1.28 ± 0.22 |

T/Bl, T/M, P/Bl and P/M Uptake Ratios

| | | | |
|---|---|---|---|
| Tumor/Blood | 2.13 | 5.52 | 6.79 |
| Tumor/Muscle | 11.44 | 25.38 | 21.62 |
| Pancreas/Blood | 10.64 | 15.96 | 7.81 |
| Pancreas/Muscle | 57.14 | 73.40 | 24.87 |

^{a}Each value in the table represents the mean and SD from 5 animals in each group.
^{b}% ID in the blood estimated assuming the whole blood volume is 6:5% of the body weight.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims the invention may be practiced otherwise than as specifically describe.

Throughout this application, various publications are referenced by citation and number. Full citations for the publication are listed below. the disclosure of these publications in their entireties are hereby incorporated by reference into this application in order to more fully describe the state of the art to which this invention pertains.

TABLE 1
Binding Affinity of Rh-BBN-37 for GRP Receptors
Expressed on Neoplasms

| Type of Cancer | Cell Line | IC₅₀ (Mean Value) |
|---|---|---|
| Pancreatic CA | CF PAC1 | 3.2 × 10⁻⁹ |
| Prostate CA | PC-3 | 7.0 × 10⁻⁹ |

TABLE 2
(% Dose)

| | Complex | | |
|---|---|---|---|
| | ¹⁰⁵Rh-Peptide22 | ¹⁰⁵Rh-Peptide22 | ¹⁰⁵Rh-Peptide22 |
| Organ | 30 min | 1 hr | 2 hr |
| (% Dose) | n = 9 | n = 9 | n = 9 |
| Brain | 0.08 ± 0.02 | 0.04 ± 0.01 | 0.06 ± 0.09 |
| Blood | 4.48 ± 1.24 | 1.86 ± 0.38 | 0.99 ± 0.34 |
| Heart | 0.13 ± 0.03 | 0.08 ± 0.03 | 0.04 ± 0.04 |
| Lung | 0.25 ± 0.08 | 0.20 ± 0.09 | 0.15 ± 0.09 |
| Liver | 7.97 ± 2.85 | 8.51 ± 2.33 | 8.57 ± 2.04 |
| Spleen | 0.07 ± 0.03 | 0.09 ± 0.08 | 0.05 ± 0.01 |
| Stomach | 1.11 ± 0.76 | 0.59 ± 0.21 | 0.30 ± 0.16 |
| Large Intestine | 0.73 ± 0.16 | 3.21 ± 3.38 | 8.91 ± 3.79 |
| Small Intestine | 6.29 ± 1.87 | 6.98 ± 1.87 | 3.48 ± 1.78 |
| Kidneys | 4.25 ± 1.33 | 3.25 ± 0.60 | 2.44 ± 0.64 |
| Bladder | 44.66 ± 7.29 | 62.88 ± 3.84 | 68.41 ± 6.63 |
| Muscle | 0.06 ± 0.03 | 0.03 ± 0.03 | 0.01 ± 0.01 |
| Pancreas | 0.95 ± 0.46 | 1.15 ± 0.49 | 1.01 ± 0.14 |
| Carcass | 32.90 ± 6.61 | 12.62 ± 4.77 | 6.37 ± 1.17 |

(% Dose/Gm)

| | Complex | | |
|---|---|---|---|
| | ¹⁰⁵Rh-Peptide22 | ¹⁰⁵Rh-Peptide22 | ¹⁰⁵Rh-Peptide22 |
| Organ | 30 min | 1 hr | 2 hr |
| (% D/GM) | n = 9 | n = 9 | n = 9 |
| Brain | 0.21 ± 0.07 | 0.14 ± 0.08 | 0.16 ± 0.28 |
| Blood | 2.22 ± 0.40 | 1.02 ± 0.22 | 0.51 ± 0.11 |
| Heart | 0.92 ± 0.25 | 0.64 ± 0.20 | 0.38 ± 0.33 |
| Lung | 1.44 ± 0.33 | 1.24 ± 0.54 | 0.92 ± 0.69 |
| Liver | 4.33 ± 1.52 | 5.18 ± 1.52 | 5.17 ± 1.12 |
| Spleen | 0.86 ± 0.38 | 1.10 ± 0.65 | 0.84 ± 0.53 |
| Stomach | 2.46 ± 1.65 | 1.53 ± 0.74 | 0.71 ± 0.33 |
| Large Intestine | 0.78 ± 0.19 | 4.42 ± 4.62 | 10.10 ± 4.58 |
| Small Intestine | 4.73 ± 1.47 | 5.84 ± 1.81 | 2.86 ± 1.47 |
| Kidneys | 7.57 ± 1.49 | 6.70 ± 0.75 | 4.60 ± 0.83 |
| Muscle | 0.53 ± 0.32 | 0.61 ± 0.97 | 0.24 ± 0.24 |
| Pancreas | 3.12 ± 0.99 | 4.31 ± 1.98 | 3.88 ± 1.25 |

TABLE 3
(% Dose)

| | Complex | | |
|---|---|---|---|
| | ¹⁰⁵Rh-Pept37 | ¹⁰⁵Rh-Pept37 | ¹⁰⁵Rh-Pept37 |
| | 30 min | 1 hr | 2 hr |
| Organ (% Dose) | n = 5 | n = 9 | n = 7 |
| Brain | 0.03 ± 0.01 | 0.07 ± 0.11 | 0.03 ± 0.03 |
| Blood | 3.09 ± 0.54 | 1.46 ± 0.62 | 0.66 ± 0.26 |
| Heart | 0.12 ± 0.03 | 0.05 ± 0.03 | 0.04 ± 0.02 |
| Lung | 0.26 ± 0.09 | 0.12 ± 0.07 | 0.08 ± 0.11 |
| Liver | 13.04 ± 1.93 | 13.00 ± 3.59 | 10.12 ± 1.86 |
| Spleen | 0.21 ± 0.13 | 0.16 ± 0.08 | 0.10 ± 0.04 |
| Stomach | 0.80 ± 0.34 | 0.65 ± 0.52 | 0.83 ± 0.96 |
| Large Intestine | 2.05 ± 0.69 | 2.96 ± 1.67 | 8.07 ± 2.25 |
| Small Intestine | 8.44 ± 1.89 | 11.38 ± 3.02 | 5.04 ± 2.27 |
| Kidneys | 7.82 ± 2.52 | 6.04 ± 1.68 | 4.57 ± 1.29 |
| Bladder | 39.65 ± 7.21 | 51.82 ± 7.53 | 62.32 ± 5.78 |
| Muscle | 0.06 ± 0.03 | 0.02 ± 0.01 | 0.02 ± 0.02 |
| Pancreas | 2.73 ± 1.14 | 3.63 ± 1.22 | 2.25 ± 1.02 |
| Carcass | 24.35 ± 7.69 | 9.81 ± 2.91 | 6.37 ± 1.73 |

(% Dose/Gm)

| | Complex | | |
|---|---|---|---|
| | ¹⁰⁵Rh-Pept37 | ¹⁰⁵Rh-Pept37 | ¹⁰⁵Rh-Pept37 |
| | 30 min | 1 hr | 2 hr |
| Organ (% D/GM) | n = 5 | n = 9 | n = 7 |
| Brain | 0.10 ± 0.05 | 0.26 ± 0.41 | 0.10 ± 0.09 |
| Blood | 1.60 ± 0.30 | 0.72 ± 0.31 | 0.34 ± 0.15 |
| Heart | 0.92 ± 0.26 | 0.38 ± 0.21 | 0.28 ± 0.17 |
| Lung | 1.52 ± 0.48 | 0.76 ± 0.47 | 0.46 ± 0.50 |
| Liver | 7.31 ± 1.15 | 7.65 ± 1.29 | 6.30 ± 1.73 |
| Spleen | 2.18 ± 1.17 | 1.59 ± 0.71 | 1.05 ± 0.44 |
| Stomach | 1.53 ± 0.67 | 1.63 ± 1.17 | 2.18 ± 2.35 |
| Large Intestine | 2.46 ± 0.70 | 3.80 ± 2.42 | 11.84 ± 4.39 |
| Small Intestine | 5.69 ± 1.26 | 7.85 ± 1.87 | 3.81 ± 2.01 |
| Kidneys | 14.28 ± 2.84 | 11.21 ± 3.68 | 8.39 ± 2.36 |
| Muscle | 0.73 ± 0.39 | 0.20 ± 0.14 | 0.39 ± 0.38 |
| Pancreas | 14.02 ± 3.23 | 15.54 ± 6.21 | 9.91 ± 5.35 |

TABLE 4
ES-MS and HPLC data of DOTA-BBN[7-14]NH₂ and In-DOTA-
BBN[7-14]NH₂ analogues.

| | ES-MS | | | HPLC |
|---|---|---|---|---|
| BBN Analogue | Mol. Formula | Calculated | Observed | tᵣ (min)^{a} |
| 0 | C₅₉H₉₁N₁₇O₁₆S | 1326.5 | 1326.6 | 13.2 |
| 3 | C₆₂H₉₆N₁₈O₁₇S | 1397.6 | 1397.4 | 13.4 |
| 5 | C₆₄H₁₀₀N₁₈O₁₇S | 1425.7 | 1425.8 | 14.0 |
| 8 | C₆₇H₁₀₆N₁₈O₁₇S | 1467.8 | 1467.8 | 19.1 |
| 11 | C₇₀H₁₁₂N₁₈O₁₇S | 1509.8 | 1509.8 | 17.1^{b} |
| In-0 | C₅₉H₈₈N₁₇O₁₆SIn | 1438.3 | 1438.2 | 12.9 |
| In-3 | C₆₂H₉₃N₁₈O₁₇SIn | 1509.4 | 1509.6 | 12.7 |
| In-5 | C₆₄H₉₇N₁₈O₁₇SIn | 1536.5 | 1537.7 | 13.6 |
| In-8 | C₆₇H₁₀₃N₁₈O₁₇SIn | 1579.6 | 1579.7 | 19.0 |
| In-11 | C₇₀H₁₀₉N₁₈O₁₇SIn | 1621.6 | 1621.7 | 16.8^{b} |

TABLE 5
IC₅₀ (nM) values (n = 3 or 4 separate experiments performed
in duplicate) of In-DOTA-BBN[7-14]NH₂ analogues vs.
¹²⁵I-Tyr⁴-BBN in human prostate PC-3 cells and human
breast carcinoma T47D cells.

| | PC-3 | T47D |
|---|---|---|
| BBN Analogue | IC₅₀ (nM) | IC₅₀ (nM) |
| 0 | 110.6 ± 32.3 | 322 ± 54.5 |
| β-Ala | 2.1 ± 0.3 | 4.7 ± 0.7 |
| 5-Ava | 1.7 ± 0.4 | 2.3 ± 1.01 |
| 8-Aoc | 0.6 ± 0.1 | 1.3 ± 0.21 |
| 11-Aun | 64.0 ± 11.2 | 516 ± 32.2 |

TABLE 6
¹¹¹In-DOTA-SPACER-BBN[7-14]NH₂ biodistribution (Avg % ID/gm, n = 5) in
CF1 normal mice after 1 hour post-injection.

| Spacer | | | | | |
|---|---|---|---|---|---|
| Tissue | 0 | β-Ala | 5-Ava | 8-Aoc | 11-Aun |
| Blood | 0.10 ± 0.03 | 0.11 ± 0.06 | 0.20 ± 0.07 | 0.32 ± 0.09 | 0.34 ± 0.08 |
| Heart | 0.05 ± 0.02 | 0.06 ± 0.04 | 0.10 ± 0.04 | 0.05 ± 0.02 | 0.13 ± 0.04 |
| Lung | 0.13 ± 0.03 | 0.11 ± 0.08 | 0.20 ± 0.06 | 0.31 ± 0.07 | 0.26 ± 0.05 |
| Liver | 0.09 ± 0.01 | 0.11 ± 0.02 | 0.16 ± 0.02 | 0.65 ± 0.07 | 1.22 ± 0.25 |
| Spleen | 0.08 ± 0.02 | 0.37 ± 0.06 | 0.87 ± 0.28 | 1.51 ± 0.41 | 1.15 ± 0.38 |
| Stomach | 0.06 ± 0.03 | 0.30 ± 0.07 | 0.71 ± 0.24 | 1.02 ± 0.26 | 1.05 ± 0.25 |
| L. Intestine | 0.09 ± 0.03 | 1.10 ± 0.78 | 3.07 ± 0.86 | 2.66 ± 1.07 | 4.34 ± 1.34 |
| S. Intestine | 0.44 ± 0.64 | 1.01 ± 0.37 | 3.49 ± 0.87 | 4.43 ± 0.90 | 11.12 ± 2.07 |
| Kidney | 1.24 ± 0.14 | 1.40 ± 0.27 | 1.84 ± 0.44 | 2.37 ± 0.31 | 2.06 ± 0.31 |
| Muscle | 0.03 ± 0.02 | 0.03 ± 0.02 | 0.05 ± 0.02 | 0.12 ± 0.05 | 0.09 ± 0.03 |
| Pancreas | 0.20 ± 0.04 | 4.92 ± 0.37 | 15.78 ± 2.54 | 26.97 ± 3.97 | 26.00 ± 3.46 |

TABLE 7
¹¹¹In-DOTA-SPACER-BBN[7-14]NH₂ biodistribution (Avg % ID, n = 5)
in CF1 normal mice after 1 hour post-injection.

| Spacer | | | | | |
|---|---|---|---|---|---|
| Tissue | 0 | β-Ala | 5-Ava | 8-Aoc | 11-Aun |
| Blood | 0.22 ± 0.07 | 0.23 ± 0.10 | 0.45 ± 0.14 | 0.66 ± 0.13 | 0.79 ± 0.20 |
| Heart | 0.01 ± 0.00 | 0.01 ± 0.01 | 0.02 ± 0.01 | 0.01 ± 0.00 | 0.02 ± 0.01 |
| Lung | 0.03 ± 0.00 | 0.03 ± 0.02 | 0.04 ± 0.01 | 0.07 ± 0.02 | 0.08 ± 0.01 |
| Liver | 0.17 ± 0.02 | 0.17 ± 0.03 | 0.26 ± 0.03 | 1.02 ± 0.08 | 2.44 ± 0.50 |
| Spleen | 0.01 ± 0.00 | 0.05 ± 0.00 | 0.11 ± 0.04 | 0.17 ± 0.04 | 0.19 ± 0.04 |
| Stomach | 0.03 ± 0.01 | 0.13 ± 0.02 | 0.37 ± 0.16 | 0.50 ± 0.06 | 0.53 ± 0.11 |
| L. Intestine | 0.10 ± 0.02 | 0.90 ± 0.57 | 2.74 ± 0.80 | 3.02 ± 0.33 | 5.54 ± 2.42 |
| S. Intestine | 0.25 ± 0.04 | 1.57 ± 0.65 | 0.11 ± 0.04 | 6.58 ± 1.10 | 17.84 ± 1.40 |
| Kidney | 0.57 ± 0.02 | 0.62 ± 0.10 | 0.75 ± 0.14 | 1.04 ± 0.12 | 1.07 ± 0.17 |
| Urine | 96.95 ± 0.37 | 92.41 ± 0.90 | 81.29 ± 1.32 | 71.61 ± 1.82 | 53.26 ± 0.90 |
| Muscle | 0.01 ± 0.00 | 0.01 ± 0.01 | 0.01 ± 0.01 | 0.02 ± 0.01 | 0.02 ± 0.01 |
| Pancreas | 0.07 ± 0.01 | 1.84 ± 0.35 | 5.57 ± 0.99 | 10.81 ± 0.78 | 11.56 ± 1.14 |
| Carcass | 1.78 ± 0.37 | 2.23 ± 0.36 | 3.15 ± 0.70 | 5.01 ± 0.47 | 7.35 ± 1.57 |

TABLE 8
¹¹¹In-DOTA-BBN[7-14]NH₂ analogues
biodistribution (Avg % ID/gm, n = 5) in CF1
normal mice after 1 hour post-injection.

| Analogue | | 8-Aoc |
|---|---|---|
| Tissue | 8-Aoc | Blocking |
| Blood | 0.32 ± 0.09 | 0.49 ± 0.15 |
| Heart | 0.05 ± 0.02 | 0.16 ± 0.06 |
| Lung | 0.31 ± 0.07 | 0.74 ± 0.17 |
| Liver | 9.65 ± 0.07 | 0.54 ± 0.13 |
| Spleen | 1.51 ± 0.41 | 0.15 ± 0.16 |
| Stomach | 1.02 ± 0.26 | 0.32 ± 0.34 |
| L. Intestine | 2.66 ± 1.07 | 0.16 ± 0.06 |
| S. Intestine | 4.43 ± 0.90 | 0.95 ± 0.18 |
| Kidney | 2.37 ± 0.31 | 2.19 ± 0.47 |
| Muscle | 0.12 ± 0.05 | 0.11 ± 0.07 |
| Pancreas | 26.97 ± 3.97 | 0.43 ± 0.10 |

TABLE 9
¹¹¹In-DOTA-BBN[7-14]NH₂ analogues
biodistribution (Avg % ID, n = 5) in CF1
normal mice after 1 hour post-injection.

| Analogue | | 8-Aoc |
|---|---|---|
| Tissue | 8-Aoc | Blocking |
| Blood | 0.66 ± 0.13 | 0.98 ± 0.23 |
| Heart | 0.01 ± 0.00 | 0.03 ± 0.01 |
| Lung | 0.07 ± 0.02 | 0.17 ± 0.05 |
| Liver | 1.02 ± 0.08 | 0.87 ± 0.10 |
| Spleen | 0.17 ± 0.04 | 0.02 ± 0.03 |
| Stomach | 0.50 ± 0.06 | 0.16 ± 0.12 |
| L. Intestine | 3.02 ± 0.33 | 0.15 ± 0.05 |
| S. Intestine | 6.58 ± 1.10 | 1.65 ± 0.19 |
| Kidney | 1.04 ± 0.12 | 0.92 ± 0.13 |
| Urine | 71.61 ± 1.82 | 88.19 ± 1.79 |
| Muscle | 0.02 ± 0.01 | 0.02 ± 0.01 |
| Pancreas | 10.81 ± 0.78 | 0.19 ± 0.03 |
| Carcass | 5.01 ± 0.47 | 7.42 ± 1.35 |

TABLE 10
¹¹¹In-DOTA-8-Aoc-BBN[7-14]NH₂ biodistribution
(Avg % ID/gm, n = 5) in PC-3 tumor bearing mice.

| | Time | | | | | | |
|---|---|---|---|---|---|---|---|
| Tissue | 15 min | 30 min | 1 hr | 4 hrs | 24 hrs | 48 hrs | 72 hrs |
| Blood | 5.585 ± 2.43 | 1.46 ± 0.44 | 0.60 ± 0.39 | 0.27 ± 0.02 | 0.10 ± 0.03 | 0.07 ± 0.03 | 0.01 ± 0.02 |
| Heart | 2.20 ± 1.05 | 0.62 ± 0.33 | 0.25 ± 0.18 | 0.13 ± 0.06 | 0.05 ± 0.09 | 0.05 ± 0.05 | 0.01 ± 0.01 |
| Lung | 3.35 ± 1.22 | 0.94 ± 0.28 | 0.50 ± 0.39 | 0.25 ± 0.08 | 0.09 ± 0.07 | 0.06 ± 0.02 | 0.02 ± 0.02 |
| Liver | 2.03 ± 0.85 | 0.70 ± 0.21 | 1.34 ± 0.25 | 1.44 ± 0.57 | 0.37 ± 0.12 | 0.13 ± 0.04 | 0.07 ± 0.02 |
| Spleen | 2.21 ± 0.80 | 0.83 ± 0.26 | 1.39 ± 1.17 | 1.59 ± 0.27 | 0.46 ± 0.20 | 0.22 ± 0.22 | 0.08 ± 0.09 |
| Stomach | 3.30 ± 1.99 | 1.82 ± 0.44 | 1.99 ± 0.24 | 0.96 ± 0.57 | 0.30 ± 0.05 | 0.12 ± 0.03 | 0.05 ± 0.02 |
| L. Intestine | 8.58 ± 3.04 | 4.33 ± 0.44 | 4.29 ± 2.55 | 10.27 ± 2.70 | 2.35 ± 0.43 | 0.81 ± 0.20 | 0.45 ± 0.04 |
| S. Intestine | 7.82 ± 2.26 | 5.16 ± 1.06 | 6.80 ± 1.81 | 2.24 ± 0.35 | 0.89 ± 0.16 | 0.25 ± 0.06 | 0.12 ± 0.02 |
| Kidney | 29.03 ± 14.40 | 8.70 ± 2.80 | 5.66 ± 1.33 | 3.18 ± 0.43 | 1.18 ± 0.14 | 0.48 ± 0.09 | 0.20 ± 0.02 |
| Muscle | 1.30 ± 0.60 | 0.32 ± 0.12 | 0.08 ± 0.07 | 0.04 ± 0.02 | 0.05 ± 0.05 | 0.02 ± 0.04 | 0.01 ± 0.02 |
| Pancreas | 54.33 ± 9.70 | 27.87 ± 3.44 | 18.80 ± 10.97 | 16.55 ± 4.43 | 6.78 ± 1.15 | 0.77 ± 0.44 | 0.23 ± 0.08 |
| Tumor | 7.59 ± 2.11 | 4.58 ± 0.53 | 3.63 ± 1.11 | 1.78 ± 1.09 | 1.56 ± 0.45 | 0.68 ± 0.24 | 0.34 ± 0.10 |

TABLE 11
¹¹In-DOTA-8-Aoc-BBN[7-14]NH₂ biodistribution
(Avg % ID, n = 5) in PC-3 tumor bearing mice.

| | Time | | | | | | |
|---|---|---|---|---|---|---|---|
| Tissue | 15 min | 30 min | 1 hr | 4 hrs | 24 hrs | 48 hrs | 72 hrs |
| Blood | 7.92 ± 2.03 | 2.47 ± 0.74 | 0.92 ± 0.58 | 0.40 ± 0.11 | 0.15 ± 0.04 | 0.12 ± 0.05 | 0.02 ± 0.03 |
| Heart | 0.20 ± 0.07 | 0.06 ± 0.02 | 0.03 ± 0.02 | 0.01 ± 0.01 | 0.00 ± 0.01 | 0.01 ± 0.01 | 0.00 ± 0.00 |
| Lung | 0.62 ± 0.25 | 0.20 ± 0.06 | 0.09 ± 0.07 | 0.05 ± 0.02 | 0.02 ± 0.01 | 0.01 ± 0.00 | 0.00 ± 0.00 |
| Liver | 1.85 ± 0.41 | 0.85 ± 0.21 | 1.41 ± 0.32 | 1.57 ± 0.72 | 0.40 ± 0.17 | 0.15 ± 0.04 | 0.08 ± 0.01 |
| Spleen | 0.10 ± 0.02 | 0.08 ± 0.03 | 0.09 ± 0.07 | 0.10 ± 0.02 | 0.03 ± 0.02 | 0.01 ± 0.01 | 0.01 ± 0.01 |
| Stomach | 0.98 ± 0.18 | 0.65 ± 0.04 | 0.52 ± 0.11 | 0.34 ± 0.23 | 0.09 ± 0.02 | 0.06 ± 0.02 | 0.02 ± 0.01 |
| L. Intestine | 5.84 ± 0.90 | 4.53 ± 0.45 | 2.18 ± 0.86 | 6.04 ± 2.05 | 1.46 ± 0.42 | 0.73 ± 0.21 | 0.41 ± 0.05 |
| S. Intestine | 6.98 ± 0.35 | 6.24 ± 0.46 | 7.45 ± 1.62 | 2.43 ± 0.56 | 0.98 ± 0.26 | 0.32 ± 0.07 | 0.15 ± 0.02 |
| Kidney | 7.18 ± 3.16 | 2.45 ± 0.78 | 1.81 ± 0.37 | 0.97 ± 0.08 | 0.36 ± 0.08 | 0.16 ± 0.04 | 0.06 ± 0.01 |
| Urine | 27.91 ± 10.33 | 62.61 ± 5.02 | 68.56 ± 6.96 | 81.83 ± 3.82 | 87.15 ± 4.31 | 91.75 ± 4.13 | 92.53 ± 1.09 |
| Muscle | 0.16 ± 0.06 | 0.05 ± 0.02 | 0.01 ± 0.01 | 0.0 ± 0.00 | 0.01 ± 0.01 | 0.00 ± 0.00 | 0.00 ± 0.00 |
| Feces | — | — | — | — | 6.10 ± 2.60 | 5.89 ± 3.81 | 6.23 ± 1.00 |
| Pancreas | 10.26 ± 1.44 | 7.37 ± 1.20 | 3.49 ± 2.15 | 3.28 ± 0.80 | 1.19 ± 0.49 | 0.17 ± 0.09 | 0.06 ± 0.02 |
| Carcass | 33.06 ± 5.51 | 12.31 ± 3.27 | 13.55 ± 6.05 | 2.92 ± 0.64 | 1.69 ± 0.56 | 0.57 ± 0.02 | 0.38 ± 0.06 |
| Tumor | 1.92 ± 1.22 | 0.99 ± 0.62 | 0.36 ± 0.32 | 0.18 ± 0.17 | 0.25 ± 0.13 | 0.08 ± 0.05 | 0.03 ± 0.01 |

TABLE 12
⁹⁰Y-DOTA-8-Aoc-BBN[7-14]NH₂ biodistribution
(Avg % ID/gm) in PC-3 tumor bearing mice.

| | Time | | | | |
|---|---|---|---|---|---|
| | 1 hr | 4 hrs | 24 hrs | 48 hrs | 72 hrs |
| Tissue | (n = 9) | (n = 9) | (n = 6) | (n = 6) | (n = 6) |
| Blood | 0.34 ± 0.12 | 0.05 ± 0.07 | 0.07 ± 0.08 | 0.06 ± 0.08 | 0.06 ± 0.09 |
| Heart | 0.10 ± 0.11 | 0.10 ± 0.14 | 0.00 ± 0.00 | 0.14 ± 0.19 | 0.36 ± 0.45 |
| Lung | 0.22 ± 0.12 | 0.07 ± 0.07 | 0.01 ± 0.02 | 0.03 ± 0.03 | 0.02 ± 0.03 |
| Liver | 0.39 ± 0.29 | 0.18 ± 0.12 | 0.08 ± 0.03 | 0.03 ± 0.03 | 0.08 ± 0.11 |
| Spleen | 1.09 ± 0.67 | 0.35 ± 0.42 | 0.11 ± 0.13 | 0.09 ± 0.24 | 0.28 ± 0.26 |
| Stomach | 1.34 ± 0.64 | 0.55 ± 0.16 | 0.09 ± 0.07 | 0.04 ± 0.07 | 0.07 ± 0.03 |
| L. Intestine | 3.35 ± 1.12 | 5.17 ± 1.85 | 1.27 ± 0.92 | 0.77 ± 0.15 | 0.47 ± 0.24 |
| S. Intestine | 3.64 ± 0.82 | 1.66 ± 0.91 | 0.35 ± 0.16 | 0.13 ± 0.04 | 0.08 ± 0.05 |
| Kidney | 3.77 ± 1.41 | 1.68 ± 0.76 | 0.51 ± 0.25 | 0.29 ± 0.12 | 0.43 ± 0.29 |
| Muscle | 0.15 ± 0.19 | 0.07 ± 0.13 | 0.02 ± 0.03 | 0.07 ± 0.13 | 0.08 ± 0.19 |
| Pancreas | 24.73 ± 4.97 | 14.02 ± 4.89 | 1.80 ± 0.57 | 0.59 ± 0.14 | 0.27 ± 0.17 |
| Tumor | 2.95 ± 0.99 | 1.98 ± 0.66 | 1.08 ± 0.37 | 0.58 ± 0.30 | 0.46 ± 0.48 |

TABLE 13
⁹⁰Y-DOTA-8-Aoc-BEN[7-14]NH₂ biodistribution
(Avg % ID) in PC-3 tumor bearing mice.

| | Time | | | | |
|---|---|---|---|---|---|
| | 1 hr | 4 hrs | 24 hrs | 48 hrs | 72 hrs |
| Tissue | (n = 9) | (n = 9) | (n = 6) | (n = 6) | (n = 6) |
| Blood | 0.56 ± 0.20 | 0.09 ± 0.13 | 0.12 ± 0.14 | 0.10 ± 0.14 | 0.11 ± 0.15 |
| Heart | 0.01 ± 0.01 | 0.01 ± 0.01 | 0.00 ± 0.00 | 0.02 ± 0.02 | 0.04 ± 0.04 |
| Lung | 0.06 ± 0.03 | 0.01 ± 0.01 | 0.00 ± 0.00 | 0.01 ± 0.01 | 0.00 ± 0.01 |
| Liver | 0.44 ± 0.34 | 0.19 ± 0.13 | 0.10 ± 0.03 | 0.04 ± 0.04 | 0.08 ± 0.11 |
| Spleen | 0.07 ± 0.04 | 0.02 ± 0.03 | 0.01 ± 0.01 | 0.01 ± 0.02 | 0.02 ± 0.01 |
| Stomach | 0.41 ± 0.12 | 0.18 ± 0.06 | 0.04 ± 0.04 | 0.01 ± 0.02 | 0.04 ± 0.02 |
| L. Intestine | 2.44 ± 0.66 | 3.33 ± 0.71 | 1.11 ± 0.75 | 0.47 ± 0.12 | 0.35 ± 0.20 |
| S. Intestine | 4.65 ± 0.98 | 2.06 ± 1.27 | 0.51 ± 0.20 | 0.16 ± 0.06 | 0.11 ± 0.07 |
| Kidney | 1.22 ± 0.46 | 0.54 ± 0.25 | 0.17 ± 0.09 | 0.10 ± 0.04 | 0.13 ± 0.07 |
| Urine | 57.73 ± 14.52 | 67.02 ± 16.74 | 67.62 ± 17.26 | 76.74 ± 21.06 | 82.97 ± 25.39 |
| Muscle | 0.02 ± 0.02 | 0.01 ± 0.01 | 0.00 ± 0.01 | 0.01 ± 0.02 | 0.01 ± 0.02 |
| Feces | — | — | 10.71 ± 8.29 | 6.78 ± 3.88 | 13.89 ± 4.48 |
| Pancreas | 5.70 ± 1.60 | 3.42 ± 0.82 | 0.44 ± 0.08 | 0.15 ± 0.04 | 0.08 ± 0.05 |
| Carcass | 0.62 ± 0.44 | 0.14 ± 0.11 | 0.04 ± 0.04 | 0.11 ± 0.17 | 0.10 ± 0.11 |
| Tumor | 0.40 ± 0.22 | 0.34 ± 0.22 | 0.16 ± 0.09 | 0.11 ± 0.07 | 0.06 ± 0.06 |

TABLE 14
In Vivo Biodistribution Analyses (% ID/g
(SD), n = 5) of ¹¹¹In-DOTA-8-Aoc-BBN[7-14]NH₂ in
Tumor-Bearing Mice Models (MDA-MB-231).

| Tissue/Organ | 1 hour | 4 hours | 24 hours |
|---|---|---|---|
| Blood | 0.35 ± 0.08 | 0.08 ± 0.10 | 0.02 ± 0.03 |
| Heart | 0.15 ± 0.11 | 0.03 ± 0.05 | 0.08 ± 0.06 |
| Lung | 0.31 ± 0.09 | 0.06 ± 0.06 | 0.05 ± 0.05 |
| Liver | 0.31 ± 0.04 | 0.15 ± 0.09 | 0.07 ± 0.02 |
| Spleen | 0.57 ± 0.10 | 0.48 ± 0.25 | 0.21 ± 0.07 |
| Stomach | 1.49 ± 0.68 | 0.27 ± 0.08 | 0.33 ± 0.10 |
| L. Intestine | 5.14 ± 0.42 | 5.58 ± 1.26 | 2.76 ± 0.49 |
| S. Intestine | 5.15 ± 0.19 | 1.52 ± 0.19 | 0.90 ± 0.14 |
| Kidney | 3.29 ± 0.56 | 1.76 ± 0.15 | 0.98 ± 0.28 |
| Pancreas | 23.4 ± 4.99 | 17.9 ± 5.00 | 5.06 ± 0.77 |
| Muscle | 0.08 ± 0.05 | 0.06 ± 0.13 | 0.03 ± 0.05 |
| Tumor 1 | 0.91 ± 0.16 | 0.36 ± 0.13 | 0.22 ± 0.07 |
| Tumor 2 | 0.74 ± 0.27 | 0.40 ± 0.23 | 0.24 ± 0.15 |
| Urine (% ID) | 72.1 ± 3.55 | 84.3 ± 2.09 | 83.8 ± 1.41 |

TABLE 15
¹⁴⁹Pm-DOTA-SPACER-BBN[7-14]NH₂ biodistribution
(Avg % ID/gm, n = 5) in CF1 normal mice after 1 hour post-injection.

| | Spacer | | | |
|---|---|---|---|---|
| Tissue | 0 | β-Ala | 5-Ava | 8-Aoc |
| Blood | 0.00 ± 0.00 | 0.21 ± 0.22 | 0.27 ± 0.06 | 0.12 ± 0.13 |
| Heart | 0.00 ± 0.00 | 0.17 ± 0.24 | 0.42 ± 0.59 | 0.03 ± 0.06 |
| Lung | 0.00 ± 0.00 | 0.34 ± 0.30 | 0.78 ± 1.08 | 0.09 ± 0.14 |
| Liver | 0.12 ± 0.10 | 0.15 ± 0.05 | 0.23 ± 0.13 | 0.19 ± 0.12 |
| Spleen | 0.00 ± 0.00 | 0.16 ± 0.31 | 2.37 ± 1.36 | 1.61 ± 0.36 |
| Stomach | 0.04 ± 0.08 | 0.19 ± 0.11 | 1.90 ± 1.60 | 1.16 ± 0.59 |
| L. Intestine | 0.01 ± 0.03 | 0.42 ± 0.08 | 3.53 ± 1.10 | 4.14 ± 2.14 |
| S. Intestine | 0.27 ± 0.17 | 0.63 ± 0.20 | 5.15 ± 1.20 | 12.56 ± 16.70 |
| Kidney | 1.04 ± 0.90 | 2.05 ± 1.63 | 2.81 ± 0.66 | 3.74 ± 1.02 |
| Muscle | 0.00 ± 0.00 | 0.04 ± 0.10 | 0.24 ± 0.25 | 0.09 ± 0.21 |
| Pancreas | 0.00 ± 0.00 | 2.40 ± 1.33 | 22.1 ± 5.40 | 28.29 ± 13.26 |

TABLE 16
¹⁴⁹Pm-DOTA-SPACER-BBN[7-14]NH₂ biodistribution (Avg % ID, n = 5)
in CF1 normal mice after 1 hour post-injection.

| | Spacer | | | |
|---|---|---|---|---|
| Tissue | 0 | β-Ala | 5-Ava | 8-Aoc |
| Blood | 0.00 ± 0.00 | 0.30 ± 0.32 | 0.47 ± 0.11 | 0.23 ± 0.26 |
| Heart | 0.00 ± 0.00 | 0.02 ± 0.02 | 0.06 ± 0.09 | 0.00 ± 0.01 |
| Lung | 0.00 ± 0.00 | 0.06 ± 0.04 | 0.17 ± 0.21 | 0.02 ± 0.04 |
| Liver | 0.16 ± 0.15 | 0.23 ± 0.07 | 0.37 ± 0.20 | 0.35 ± 0.20 |
| Spleen | 0.00 ± 0.00 | 0.02 ± 0.04 | 0.27 ± 0.13 | 0.24 ± 0.06 |
| Stomach | 0.02 ± 0.05 | 0.10 ± 0.03 | 0.77 ± 0.74 | 0.66 ± 0.35 |
| L. Intestine | 0.01 ± 0.02 | 0.31 ± 0.06 | 3.18 ± 1.18 | 4.43 ± 2.37 |
| S. Intestine | 0.38 ± 0.25 | 0.95 ± 0.19 | 7.70 ± 0.66 | 7.84 ± 2.15 |
| Kidney | 0.34 ± 0.28 | 0.61 ± 0.41 | 1.11 ± 0.29 | 1.55 ± 0.47 |
| Urine | 97.10 ± 2.91 | 95.54 ± 1.15 | 75.82 ± 2.02 | 67.20 ± 5.53 |
| Muscle | 0.00 ± 0.00 | 0.00 ± 0.01 | 0.03 ± 0.04 | 0.01 ± 0.02 |
| Pancreas | 0.07 ± 0.01 | 0.46 ± 0.23 | 4.25 ± 0.43 | 7.34 ± 3.51 |
| Carcass | 1.98 ± 2.27 | 1.64 ± 0.38 | 6.16 ± 0.75 | 10.30 ± 1.84 |

TABLE 17
¹⁷⁷Lu-DOTA-SPACER-BBN[7-14]NH₂ biodistribution
(Avg % ID/gm, n = 5) in CF1 normal mice after 1 hour post-injection.

| Spacer | | | | | |
|---|---|---|---|---|---|
| Tissue | 0 | β-Ala | 5-Ava | 8-Aoc | 11-Aun |
| Blood | 0.58 ± 0.96 | 0.16 ± 0.17 | 0.22 ± 0.19 | 0.14 ± 0.10 | 0.78 ± 1.10 |
| Heart | 0.04 ± 0.09 | 0.43 ± 0.70 | 0.34 ± 0.35 | 0.19 ± 0.36 | 1.56 ± 2.40 |
| Lung | 0.19 ± 0.26 | 0.23 ± 0.33 | 0.47 ± 0.84 | 0.20 ± 0.21 | 0.73 ± 0.81 |
| Liver | 0.09 ± 0.06 | 0.15 ± 0.06 | 0.09 ± 0.04 | 0.23 ± 0.05 | 1.65 ± 0.29 |
| Spleen | 0.04 ± 0.09 | 0.31 ± 0.31 | 1.26 ± 0.69 | 1.23 ± 0.59 | 1.78 ± 1.87 |
| Stomach | 0.10 ± 0.21 | 0.34 ± 0.18 | 1.48 ± 2.25 | 1.41 ± 0.44 | 1.82 ± 1.12 |
| L. Intestine | 0.07 ± 0.09 | 0.45 ± 0.19 | 3.78 ± 1.23 | 6.17 ± 0.79 | 6.31 ± 0.86 |
| S. Intestine | 0.75 ± 0.60 | 0.49 ± 0.10 | 2.55 ± 1.31 | 6.47 ± 1.24 | 12.58 ± 1.73 |
| Kidney | 1.21 ± 0.31 | 1.88 ± 0.37 | 2.03 ± 1.02 | 4.97 ± 0.71 | 4.97 ± 0.61 |
| Muscle | 0.09 ± 0.15 | 0.94 ± 1.54 | 0.67 ± 0.90 | 0.17 ± 0.39 | 0.75 ± 1.12 |
| Pancreas | 0.18 ± 0.28 | 1.44 ± 0.26 | 16.41 ± 1.38 | 30.83 ± 1.89 | 35.48 ± 2.39 |

TABLE 18
¹⁷⁷Lu-DOTA-SPACER-BBN[7-14]NH₂ biodistribution
(Avg % ID, n = 5) in CF1 normal mice after 1 hour post-injection.

| Spacer | | | | | |
|---|---|---|---|---|---|
| Tissue | 0 | β-Ala | 5-Ava | 8-Aoc | 11-Aun |
| Blood | 0.39 ± 0.34 | 0.24 ± 0.25 | 0.35 ± 0.31 | 0.20 ± 0.15 | 0.47 ± 0.54 |
| Heart | 0.01 ± 0.02 | 0.05 ± 0.08 | 0.04 ± 0.05 | 0.02 ± 0.04 | 0.19 ± 0.29 |
| Lung | 0.04 ± 0.06 | 0.04 ± 0.06 | 0.08 ± 0.15 | 0.03 ± 0.04 | 0.17 ± 0.22 |
| Liver | 0.19 ± 0.10 | 0.21 ± 0.09 | 0.14 ± 0.06 | 0.31 ± 0.05 | 2.26 ± 0.46 |
| Spleen | 0.01 ± 0.01 | 0.05 ± 0.04 | 0.18 ± 0.12 | 0.16 ± 0.05 | 0.24 ± 0.24 |
| Stomach | 0.05 ± 0.11 | 0.13 ± 0.09 | 0.73 ± 1.33 | 0.51 ± 0.15 | 0.64 ± 0.35 |
| L. Intestine | 0.09 ± 0.12 | 0.36 ± 0.17 | 3.52 ± 1.37 | 4.63 ± 0.57 | 5.03 ± 0.46 |
| S. Intestine | 1.27 ± 1.03 | 0.64 ± 0.20 | 3.80 ± 1.87 | 9.55 ± 2.37 | 17.10 ± 3.60 |
| Kidney | 0.58 ± 0.10 | 0.63 ± 0.14 | 0.69 ± 0.33 | 1.62 ± 0.14 | 1.76 ± 0.25 |
| Urine | 93.26 ± 3.61 | 94.66 ± 1.88 | 84.08 ± 2.13 | 71.16 ± 1.05 | 58.76 ± 3.44 |
| Muscle | 0.02 ± 0.03 | 0.11 ± 0.18 | 0.09 ± 0.12 | 0.02 ± 0.05 | 0.11 ± 0.18 |
| Pancreas | 0.06 ± 0.10 | 0.32 ± 0.07 | 3.78 ± 1.09 | 7.01 ± 1.42 | 6.89 ± 1.20 |
| Carcass | 4.34 ± 2.64 | 2.73 ± 1.08 | 2.77 ± 0.75 | 4.95 ± 1.41 | 6.69 ± 2.48 |

TABLE 19
¹⁷⁷Lu-DOTA-8-Aoc-BBN[7-14]NH₂ biodistribution
(Avg % ID/gm, n = 5) in PC-3 tumor bearing mice.

| | Time | | |
|---|---|---|---|
| | 1 hr | 4 hrs | 24 hrs |
| Tissue | (n = 5) | (n = 5) | (n = 5) |
| Blood | 0.38 ± 0.22 | 0.08 ± 0.07 | 0.01 ± 0.01 |
| Heart | 0.15 ± 0.22 | 0.07 ± 0.13 | 0.06 ± 0.09 |
| Lung | 0.18 ± 0.09 | 0.11 ± 0.15 | 0.14 ± 0.26 |
| Liver | 0.30 ± 0.05 | 0.13 ± 0.02 | 0.03 ± 0.02 |
| Spleen | 0.33 ± 0.51 | 0.60 ± 0.36 | 0.08 ± 0.10 |
| Stomach | 1.38 ± 0.52 | 0.34 ± 0.34 | 0.19 ± 0.13 |
| L. Intestine | 3.29 ± 0.61 | 7.29 ± 3.73 | 1.90 ± 0.53 |
| S. Intestine | 5.60 ± 0.46 | 1.93 ± 0.96 | 0.48 ± 0.14 |
| Kidney | 4.70 ± 0.95 | 2.18 ± 0.31 | 0.60 ± 0.20 |
| Muscle | 0.11 ± 0.13 | 0.15 ± 0.21 | 0.10 ± 0.17 |
| Pancreas | 38.53 ± 3.61 | 22.18 ± 4.66 | 4.97 ± 2.28 |
| Tumor | 4.22 ± 1.09 | 3.03 ± 0.91 | 1.54 ± 1.14 |

TABLE 20
¹⁷⁷Lu-DOTA-8-Aoc-BBN[7-14]NH₂ biodistribution (Avg % ID,
n = 5) in PC-3 tumor bearing mice.

| | Time | | |
|---|---|---|---|
| | 1 hr | 4 hrs | 24 hrs |
| Tissue | (n = 5) | (n = 5) | (n = 5) |
| Blood | 0.62 + 0.44 | 0.12 + 0.11 | 0.01 + 0.02 |
| Heart | 0.01 + 0.02 | 0.01 + 0.02 | 0.01 + 0.01 |
| Lung | 0.04 + 0.02 | 0.05 + 0.09 | 0.03 + 0.05 |
| Liver | 0.38 + 0.09 | 0.15 + 0.03 | 0.04 + 0.03 |
| Spleen | 0.03 + 0.04 | 0.05 + 0.02 | 0.01 + 0.01 |
| Stomach | 0.61 + 0.09 | 0.22 + 0.06 | 0.09 + 0.06 |
| L. Intestine | 3.64 + 0.72 | 7.28 + 4.23 | 1.75 + 0.23 |
| S. Intestine | 8.20 + 1.72 | 2.51 + 0.75 | 0.67 + 0.12 |
| Kidney | 1.35 + 0.41 | 0.61 + 0.08 | 0.17 + 0.06 |
| Urine | 67.41 + 2.45 | 79.76 + 6.48 | 85.85 + 1.39 |
| Muscle | 0.01 + 0.02 | 0.02 + 0.03 | 0.02 + 0.03 |
| Pancreas | 9.70 + 1.12 | 5.23 + 1.68 | 1.31 + 0.45 |
| Tumor | 1.15 + 0.72 | 0.78 + 0.27 | 0.29 + 0.18 |
| Carcass | 6.18 + 1.01 | 2.52 + 1.18 | 2.08 + 3.14 |

### REFERENCES CITED

Albert et al., (1991) Labeled Polypeptide Derivatives, Int'l Patent No. WO91/01144.
Bjisterbosch, M. K., et al., (1995) Quarterly J. Nucl. Med. 39:4-19.
Bushbaum, (1995) Pharmacokinetics of Antibodies and Their Radiolabels. In: Cancer Therapy with Radiolabeled Antibodies, (ed) D. M. Goldenberg, CRC Press, Boca Raton, Chaper 10, 115-140 FL.
Cai et al., (1992) Peptides, 13:267.
Cai et al., (1994) Proc. Natl. Acad. Sci., 91:12664.
Coy et al., (1988) J. Biolog. Chem., 263(11), 5066.
Cutler, C., Hu, F., Hoffman, T. J., Volkert, W. A., and Jurisson, S. S., “DOTA Bombesin Complexes with Sm-153 and NCA PM-149”, The International Chemical Congress of Pacific Basin Societies, Pacifichem 2000, Honolulu, Hi., December, 2000.
Davis et al. (1992) Peptides, 13:401.
de Jong et al., (1997) Eur. J. Nucl. Med., 24:368.
Duncan et al., (1997) Cancer Res. 57:659.
Eckelman (1995) Eur. J. Nucl. Med., 22:249.
Eckelman et al., (1993) The design of site-directed radiopharmaceuticals for use in drug discovery. In: Nuclear Imaging in Drug Discovery, Development and Approval (eds) H. D. Burns et al., Birkhauser Publ. Inc., Boston, Mass.
Fischman et al., (1993) J. Nucl. Med., 33:2253.
Fritzberg et al., (1992) J. Nucl. Med., 33:394.
Frizberg et al. (1995) Radiolabeling of antibodies for targeted diagnostics. In: Targeted Delivery of Imaging Agents (ed) V. P. Torchilin, CRC Press, Boca Raton, Fla., pp. 84-101.
Gali, H., Hoffman, T. J., Owen, N. K., Sieckman, G. L., and Volkert, W. A., “In Vitro and In Vivo Evaluation of 111In_Labeled DOTA_{—}8_Aoc-BBN[7_{—}14]NH2 Conjugate for Specific Targeting of Tumors Expressing Gastrin Releasing Peptide (GRP) Receptors”, 47th Annual Meeting—Society of Nuclear Medicine, St. Louis, Mo., J. Nucl. Med., 41(5), 119P, #471, 2000.
Gali, H., Hoffman, T. J., Sieckman, G. L., Katti, K. V., and Volkert, W. A., “Synthesis, Characterization and Labeling with 99 mTc/188Re of Peptide Conjugates Containing a Dithio-Bisphosphine Chelating Agent” Bioconjugate Chemistry (Accepted), 2001.
Gali, H., Hoffman, T. J., Sieckman, G. L., Katti, K. V., and Volkert, W. A. “Synthesis, Characterization, and Labeling with 99 mTc/188Re of Peptide Conjugates Containing a Dithio-bisphosphine Chelating Agent”, American Chemical Society Annual Meeting, San Francisco, Calif., April, 2000.
Gali, H., Smith, C. J., Hoffman, T. J., Sieckman, G. L., Hayes, D. L., Owen, N. K., and Volkert, W. A., “Influence of the Radiometal on the In Vivo Pharmacokinetic Properties of a Radiometal-labeled DOTA-Conjugated Peptide”, 222nd American Chemical Society National Meeting, Chicago, Ill., August, 2001 (Accepted)
Hermanson (1996) In: Bioconjugate Techniques, Academic Press, pp. 3-136.
Hoffken, (ed) (1994) Peptides in Oncology II, Springer-Verlag, Berlin-Heidelberg.
Hoffman, et al., (1997) Quarterly J. Nucl. Med. 41(2) Supp. #1, 5.
Hoffman, T. J. and Volkert, W. A. “Design of Radioloabeled Bombesin Analogs” Receptors 2000; DOE Sponsored Workshop (La Jolla, Calif.) Apr. 17-18, 2000. Hoffman, T. J., Gali, H., Sieckman, G. L., Forte, L. R., Chin, D. T., Owen, N. K., Wooldridge, J. E., and Volkert, W. A., “Development and Characterization of a Receptor-Avid 111In-Labeled Peptide for Site—Specific Targeting of Colon Cancer”, 92nd Annual Meeting of the American Association for Cancer Research, New Orleans, La. Proceedings of the American Association for Cancer Research, Vol. 42, 139, #746, March 2001.
Hoffman, T. J., Li, N., Sieckman, G., and Volkert, W. A., “Uptake and Retention of a Rh-105 Labeled Bombesin Analogue in GRP Receptor Expressing Neoplasms: An In-Vitro Study”, 44th Annual Meeting—Society of Nuclear Medicine, June, 1997; J. Nucl. Med., 38(5), 188P, 1997.
Hoffman, T. J., Li, N., Sieckman, G., Higginbotham, C. A., and Volkert, W. A., “Evaluation of Radiolabeled (1-125 vs. Rh-105) Bombesin Analogue Internalization in Normal and Tumor Cell Lines”, 10th International Symposium on Radiopharmacology, May, 1997; Quarterly J. Nucl. Med., 41(2) Suppl#1, 5, 1997.
Hoffman, T. J., Li, N., Volkert, W. A., Sieckman, G., Higginbotham, C. A., and Ochrymowycz, L. A., “Synthesis and Characterization of Rh-105 Labeled Bombesin Analogues: Enhancement of GRP Receptor Binding Affinity Utilizing Aliphatic Carbon Chain Linkers”, J. Label. Comp'd Radiopharm., 1997.
Hoffman, T. J., Li, N., Volkert, W. A., Sieckman, G., Higginbotham, C. A., and Ochrymowycz, L. A., “Synthesis and Characterization of Rh-105 Labeled Bombesin Analogues: Enhancement of GRP Receptor Binding Affinity Utilizing Aliphatic Carbon Chain Linkers”, 12th International Symposium on Radiopharmaceutical Chemistry, June, 1997.
Hoffman, T. J., Li, N., Higginbotham, C. A., Sieckman, G., Volkert, W. A., “Specific Uptake and Retention of Rh-105 Labeled Bombesin Analogues in GRP-Receptor Expressing Cells”, European Society of Nuclear Medicine, August, 1997; Eur. J. Nucl. Med., 24(8), 901, 1997.
Hoffman, T. J., Li, N., Sieckman, G. L., Higginbotham, C. Ochrymowycz, L. A., Volkert, W. A. “Rh-105 Bombesin Analogs: Selective In Vivo Targeting of Prostate Cancer with a Therapeutic Radionuclide”, 45th Annual Meeting—Society of Nuclear Medicine, June 1998; J. Nucl. Med., 39(5), 222P, 1998.
Hoffman, T. J., Quinn, T. P., and Volkert, W. A., “Radiometallated Receptor-Avid Peptide Conjugates for Specific In Vivo Targeting of Cancer”, Nuc. Med. & Biol. (Accepted), 2001.
Hoffman, T. J., Sieckman, G., Volkert, W. A., “Targeting Small Cell Lung Cancer Using Iodinated Peptide Analogs”, J. Label. Comp'd Radiopharm., 37:321-323, 1995.
Hoffman, T. J., Sieckman, G., Volkert, W. A., “Targeting Small Cell Lung Cancer Using Iodinated Peptide Analogs”, 11th International Symposium on Radiopharmaceutical Chemistry, August, 1995; J. Label. Comp'd Radiopharm., 37:321-323, 1995.
Hoffman, T. J., Sieckman, G. L., Volkert, W. A., “Iodinated Bombesin Analogs: Effect of N-Terminal Chain Iodine Attachment on BBN/GRP Receptor Binding”, 43rd Annual Meeting—Society of Nuclear Medicine, June, 1996; J. Nucl. Med., 37(5), p185P, 1996.
Hoffman, T. J., Simpson, S. D., Smith, C. J., Sieckman, G. L., Higginbotham, C., Volkert, W. and Thornback, J. R. “Accumulation and Retention of 99 mTc-RP527 by GRP Receptor Expressing Tumors in SCID Mice”, 46th Annual Meeting—Society of Nuclear Medicine, Los Angeles, Calif., J. Nucl. Med., 40(5), 104P, 1999.
Hoffman, T. J., Simpson, S. D., Smith, C. J., Sieckman, G. L., Higginbotham, C., Eshima, D., Volkert, W. and Thornback, J. R. “Accumulation and Retention of 99 mTc-RP591 by GRP Receptor Expressing Tumors in SCID Mice”, Congress of the European Association of Nuclear Medicine, Barcelona, Spain, Eur. J. Nucl. Med., 26(9), 1157, #PS-420, September, 1999.
Hoffman, T. J., Smith, C. J., Gali, H., Owen, N. K., Sieckman, and Volkert, W. A., “In Vitro and In Vivo Evaluation of 111In/90Y Radiolabeled Peptides for Specific Targeting of Tumors Expressing Gastrin Releasing Peptide (GRP) Receptors”, 92nd Annual Meeting of the American Association for Cancer Research, New Orleans, La. Proceedings of the American Association for Cancer Research, Vol. 42, 773, #4148, March 2001.
Hoffman, T. J., Smith, C. J., Gali, H., Owen, N. K., Sieckman, Hayes, D. L., and Volkert, W. A., “Development of a Diagnostic Radiopharmaceutical for Visualization of Primary and Metastatic Breast Cancer”, 48th Annual Meeting—Society of Nuclear Medicine, Toronto, Ontario, Canada, June 2001. (Accepted)
Hoffman, T. J., Smith, C. J., Gali, H., Owen, N. K., Sieckman, Hayes, D. L., Foster, B., and Volkert, W. A., “111In/90Y Radiolabeled Peptides for Targeting Prostate Cancer; A Matched Pair Gastrin Releasing Peptide (GRP) Receptor Localizing Radiopharmaceutical”, 48th Annual Meeting-Society of Nuclear Medicine, Toronto, Ontario, Canada, June 2001. (Accepted)
Hoffman, T. J., Smith, C. J., Sieckman, G. L., Owen, N. K., and Volkert, W. A., “Design, Synthesis, and Biological Evaluation of Novel Gastrin Releasing Peptide Receptor Targeting Radiopharmaceuticals” American Chemical Society Annual Meeting, August 2000, Washington, D.C.
Hoffman, T. J., Smith, C. J., Simpson, S. D., Sieckman, G. L., Higginbotham, C., Jimenez, H., Eshima, D., Thornback, J. R., and Volkert, W. A. “Targeting Gastrin Releasing Peptide Receptor (GRP-R) Expression in Prostate and Pancreatic Cancer Using Radiolabeled GRP Agonist Peptide Vectors”, American Association for Cancer Research Annual Meeting, San Francisco, Calif., Proceedings of the American Association for Cancer Research, Vol. 41, 529, #3374, April, 2000.
Hoffman, T. J., Smith, C. J., Simpson, S. D., Sieckman, G. L., Higginbothan, C., Jimenez, H., Eshima, D., Thornback, J. R., and Volkert, W. A., “Optimizing Pharmacokinetics of Tc-99m-GRP Receptor Targeting Peptides Using Multi-Amino Acid Linking Groups”, 47th Annual Meeting—Society of Nuclear Medicine, St. Louis, Mo., J. Nucl. Med., 41(5), 228P, #1013, 2000.
Jensen et al., (1993) Rec. Result. Cancer Res., 129:87.
Karra, S. R., Schibli, R., Gali, H., Katti, K. V., Hoffman, T. J., Higginbotham, C., Sieckman, G., Volkert, W. A., “99 mTc-Labeling and In Vivo Studies of a Bombesin Analogue with a Novel Water-soluble Dithia-Diphosphine Based Bifunctional Chelating Agent”, Bioconjugate Chemistry, 10:254-260, 1999.
Katti, K. V, Gali, H., Schibli, R., Hoffman, T. J., and Volkert, W. A. “99 mTc/Re Coordination Chemistry and Biomolecule Conjugation Strategy of a Novel Water Soluble Phosphine-Based Bifunctional Chelating Agent”, Fifth International Symposium of Technetium in Chemistry and Nuclear Medicine, Bressanone, Italy, September, 1998.
Katti, K. V., Gali, H., Schibli, R., Hoffman, T. J., and Volkert, W. A. “99 mTc/Re Coordination Chemistry and Biomolecule Conjugation Strategy of a Novel Water Soluble Phosphine-Based Bifunctional Chelating Agent”, In Technetium, Rhenium and Other Metals in Chemistry and Nuclear Medicine (5), Ed. By M. Nicolini and U. Mazzi, Servizi Grafici Editoriali, Padova, pp. 93-100, 1999.
Kothari, K. K., Katti, K. V., Prabhu, K. R., Gali, H., Pillarsetty, N. K., Hoffman, T. J., Owen, N. K., and Volkert, W. A., “Development of a Diamido-Diphosphine (N2P2)-BFCA for Labeling Cancer Seeking Peptides via the 99 mTc(1)(CO)3(H2O)3 Intermediate”, 47th Annual Meeting—Society of Nuclear Medicine, St. Louis, Mo., J. Nucl. Med., 41(5), 244P, #1079, 2000.
Krenning et al., (1994) Semin. Oncology, 5-14.
Leban et al. (1994) J. Med. Chem., 37:439.
Li et al., (1996a) J. Nucl. Med., 37:61P.
Li et al., (1996b) Radiochim Acta, 75:83.
Li, W. P., Ma, D. S., Higginbotham, C., Hoffman, T. J., Ketring, A. R., and Jurisson, S. S., “Development of an In Vitro Model for Assessing the In Vivo Stability of Lanthanide Chelates”, Nuc. Med. & Biol. 28:145-154, 2001.
Lister-James et al. (1997) Quart. J. Nucl. Med., 41:111.
Lowbertz et al., (1994) Semin. Oncol., 1-5.
Mattes, (1995) Pharmacokinetics of antibodies and their radiolabels. In: Cancer Therapy with Radiolabeled Antibodies (ed) D. M. Goldenberg, CRC Press, Boca Raton, Fla.
Moody et al., (1995) Life Sciences, 56(7), 521.
Moody et al., (1996) Peptides, 17(8), 1337.
Ning, Li, Hoffman, T. J., Sieckman, G. L., Ochrymowycz, L. A., Higginbotham, C., Struttman, M., Volkert, W. A., and Ketring, A. R., “In-vitro and In-vivo Characterization of a Rh-105-tetrathiamacrocycle Conjugate of a Bombesin Analogue”, 43rd Annual Meeting—Society of Nuclear Medicine, June, 1996; J. Nucl. Med., 37(5), p61P, 1996.
Parker (1990) Chem. Soc. Rev., 19:271.
Pollak et al., (1996) Peptide Derived Radionuclide Chealtors, Int'l Patent No. WO96/03427.
Qin et al., (1994) J. Canc. Res. Clin. Oncol., 120:519.
Qin, Y. et al., (1994) Cancer Research 54: 1035-1041.
Reile, H. et al., (1994) Prostate 25: 29-38.
Schibli, R., Karra, S., Katti, K. V., Gali, H., Higginbotham, C., Sieckman, G., Hoffman, T. J., Volkert, W. A., “A Tc-99m-Dithia-Di(Bis-Hydroxy-methylene)Phosphine Conjugate of Bombesin: In Vitro and In Vivo Studies” 45th Annual Meeting—Society of Nuclear Medicine, June, 1998; J. Nucl. Med., 39(5), 225P, 1998.
Schibli, R., Karra, S. R., Gali, H., Katti, K. V., Higginbotham, C., Smith, C. J., Hoffman, T. J., and Volkert, W. A. “Conjugation of Small Biomolecules and Peptides with Water-Soluble Dithio-Bis-Hydroxymethylphosphine Ligands”, Annual Meeting of the American Chemical Society, April, 1998.
Schubiger et al., (1996) Bioconj. Chem.
Seifert et al. (1998) Appl. Radiat. Isot., 49:5.
Smith et al., (1997) Nucl. Med. Biol., 24:685.
Smith, C. J., Hoffman, T. J., Gali, H., Hayes, D. L., Owen, N. K., Sieckman, G. L., and Volkert, W. A., “Radiochemical Investigations of 177Lu-DOTA-8-Aoc-BBN(7-14)NH2: A New Gastrin Releasing Peptide Receptor (GRPr) Targeting Radiopharmaceutical”, J. Labeled Compounds and Radiopharmaceuticals (Accepted), 2001
Smith, C. J., Hoffman, T. J., Gali, H., Hayes, D. L., Owen, N. K., Sieckman, G. L., and Volkert, W. A., “Radiochemical Investigations of 177Lu-DOTA-8-Aoc-BBN(7-14)NH2: A New Gastrin Releasing Peptide Receptor (GRPr) Targeting Radiopharmaceutical”, 14th International Symposium on Radiopharmaceutical Chemistry, Interlaken Switzerland, June, 2001, J. Labeled Compounds and Radiopharmaceuticals (Accepted)
Smythe, E. et al., (1991) Eur. J. Biochem. 202: 689-699.
Troutner (1987) Nucl. Med. Biol., 14:171.
Vallabhajosula et al., (1996) J. Nucl. Med., 37:1016.
Volkert, W. A., and Hoffman, T. J. “Design and Development of Receptor-avid Peptide Conjugates for In Vivo Targeting of Cancer”, In Biomedical Imaging: Reporters, Dyes, and Instrumentation, Ed. by D. J. Bornhop, C. H. Contag, and E. M. Sevick_Muraca, Proceedings of SPIE Vol. 3600, 86-98, 1999.
Volkert, W. A., Gali, H-P, Hoffman, T. J., Owen, N. K., Sieckman, G. L., and Smith, C. J., “111In/90Y Labeled GRP Analogs: A Structure-Activity Relationship”, The International Chemical Congress of Pacific Basin Societies, Pacifichem 2000, Honolulu, Hi., December, 2000.
Volkert, W. A., Hoffman, T. J., Li, N., Sieckman, G., Higginbotham, C., “Therapeutic Potential for Small Radiometallated Site-Specific Drugs”, Radiation Research Society—Annual Meeting, May, 1997.
Wilbur (1992) Bioconj. Chem., 3:433.
Wong, E. et al., (1997) Inorg. Chem. 36: 5799-5808.
Zhu, W-Y. et al., (1991) Am. J. Physiol. 261: G57-64.

What is claimed is:

1. A compound comprising a chelating group attached to a gastrin releasing peptide (GRP) receptor agonist, wherein said compound binds a gastrin releasing peptide receptor on a cell surface and is internalized within the cell and said compound has a structure of the formula X—Y—B wherein X is a metal chelating group optionally bound to a metal, Y is a spacer group or covalent bond and B is a gastrin releasing peptide receptor agonist.

2. The compound of claim 1, wherein Y is selected from the group consisting of at least one amino acid residue, a hydrocarbon chain and a combination thereof.

3. The compound of claim 2, wherein Y is a combination of L-glutamine and a hydrocarbon chain.

4. The compound of claim 3, wherein Y is a combination of L-glutamine and a C1 to C10 hydrocarbon chain.

5. The compound of claim 2, wherein Y is selected from the group consisting of glycine, β-alanine, gamma-aminobutanoic acid, 5-aminovaleric acid (5-Ava), 6-aminohexanoic acid, 7-aminoheptanoic acid, 8-aminooctanoic acid (8-Aoc), 9-aminononanoic acid, 10-aminodecanoic acid and 11-aminoundecanoic acid (11-Aun).

6. The compound of claim 2, wherein Y is Gly-Ser-Gly.

7. The compound of claim 1, wherein X is selected from the group consisting of DOTA, DTPA, S4, N₃S, N₂S₂, NS₃ and derivatives thereof.

8. The compound of claim 7, wherein X is DOTA or a derivative thereof.

9. The compound of claim 7, wherein X is N₃S or a derivative thereof.

10. A complex comprising a metal and a compound having a structure of the formula X—Y—B, wherein X is a metal chelating group, Y is a spacer group or covalent bond and B is a gastrin releasing peptide (GRP) receptor agonist, and the metal is selected from the group consisting of transition metals, lanthanides, auger-electron emitting isotopes, and α-, β- or γ-emitting isotopes, wherein said complex binds a gastrin releasing peptide receptor on a cell surface and said complex is internalized within the cell.

11. The complex of claim 10, wherein the metal is selected from the group consisting of: ¹⁰⁵Rh—, ^{99m}Tc—, ^{186/188}Re—, ¹⁵³Sm—, ¹⁶⁶Ho—, ¹¹¹In—, ⁹⁰Y—, ¹⁷⁷Lu—, ¹⁴⁹Pm—, ¹⁶⁶Dy—, ¹⁷⁵Yb—, ¹⁹⁹Au— and ^{117m}Sn—.

12. The complex of claim 10, wherein Y is selected from the group consisting of at least one amino acid residue, a hydrocarbon chain and a combination thereof.

13. The complex of claim 12, wherein Y is a combination of L-glutamine and a hydrocarbon chain.

14. The complex of claim 12, wherein Y is a combination of L-glutamine and a C1 to C10 hydrocarbon chain.

15. The complex of claim 12, wherein Y is selected from the group consisting of glycine, β-alanine, gamma-aminobutanoic acid, 5-aminovaleric acid (5-Ava), 6-aminohexanoic acid, 7-aminoheptanoic acid, 8-aminooctanoic acid (8-Aoc), 9-aminononanoic acid, 10-aminodecanoic acid and 11-aminoundecanoic acid (11-Aun).

16. The complex of claim 15, wherein Y is 8-aminooctanoic acid.

17. The complex of claim 12, wherein Y is gly-ser-gly.

18. The complex of claim 10, wherein X is selected from the group consisting of DOTA, DTPA, S₄, N₃S, N₂S₂, NS₃ and derivatives thereof.

19. The complex of claim 18, wherein X is DOTA or a derivative thereof.

20. The complex of claim 18, wherein X is N₃S or a derivative thereof.

21. A method of treating patients using radioisotope therapy by administering an effective amount of a pharmaceutical composition comprising a compound, said compound comprising a metal complexed with a chelating group attached to a gastrin releasing peptide (GRP) receptor agonist, wherein said compound binds a gastrin releasing peptide receptor on a cell surface and is internalized within the cell and said compound has a structure of the formula X—Y—B wherein X is a metal chelating group, Y is a spacer group or covalent bond and B is a gastrin releasing peptide receptor agonist.

22. The method of claim 21, wherein the metal is selected from the group consisting of transition metals, lanthanides, auger-electron emitting isotopes, and α-, β- or γ-emitting isotopes.

23. The method of claim 22, wherein the metal is selected from the group consisting of: ¹⁰⁵Rh—, ^{186/188}Re—, ¹⁵³Sm—, ¹⁶⁶Ho—, ¹¹¹In—, ⁹⁰Y—, ¹⁷⁷Lu—, ¹⁴⁹Pm—, ¹⁶⁶Dy—, ¹⁷⁵Yb—, ⁹⁹Au— and ¹¹⁷Sn—.

24. The method of claim 21, wherein Y is selected from the group consisting of at least one amino acid residue, a hydrocarbon chain and a combination thereof.

25. The method of claim 24, wherein Y is a combination of L-glutamine and a hydrocarbon chain.

26. The method of claim 24, wherein Y is selected from the group consisting of glycine, β-alanine, gamma-aminobutanoic acid, 5-aminovaleric acid (5-Ava), 6-aminohexanoic acid, 7-aminoheptanoic acid, 8-aminooctanoic acid (8-Aoc), 9-aminononanoic acid, 10-aminodecanoic acid and 11-aminoundecanoic acid (11-Aun).

27. The method of claim 26, wherein Y is 8-aminooctanoic acid.

28. The method of claim 24, wherein Y is Gly-Ser-Gly.

29. The method of claim 21, wherein X is selected from the group consisting of DOTA, DTPA, S₄, N₃S, N₂S₂, NS₃ and derivatives thereof.

30. The method of claim 29, wherein X is DOTA or a derivative thereof.

31. The method of claim 29, wherein X is N₃S or a derivative thereof.

32. A method of imaging a patient by administering to a subject a diagnostically effective amount of a compound comprising a metal complexed with a chelating group attached to a gastrin releasing peptide (GRP) receptor agonist, wherein said compound binds a gastrin releasing peptide receptor on a cell surface and is internalized within the cell and said compound has a structure of the formula X—Y—B wherein X is a metal chelating group, Y is a spacer group or covalent bond and B is a gastrin releasing peptide receptor agonist.

33. The method of claim 32, wherein the metal is selected from the group consisting of transition metals, lanthanides, auger-electron emitting isotopes, and α-, β-, or γ-emitting isotopes.

34. The method of claim 33, wherein the metal is selected from the group consisting of: ¹⁰⁵Rh—, ^{99m}Tc—, ^{186/188}Re—, ¹⁵³Sm—, ¹⁶⁶Ho—, ¹¹¹In—, ¹⁷⁷Lu—, ¹⁴⁹Pm—, ¹⁶⁶Dy—, ¹⁷⁵Yb—, ¹⁹⁹Au— and ^{117m}Sn—.

35. The method of claim 32, wherein Y is selected is selected from the group consisting of at least one amino acid residue, a hydrocarbon chain and a combination thereof.

36. The method of claim 35, wherein Y is a combination of L-glutamine and a hydrocarbon chain.

37. The method of claim 35, wherein Y is selected from the group consisting of glycine, β-alanine, gamma-aminobutanoic acid, 5-aminovaleric acid (5-Ava), 6-aminohexanoic acid, 7-aminoheptanoic acid, 8-aminooctanoic acid (8-Aoc), 9-aminononanoic acid, 10-aminodecanoic acid and 11-aminoundecanoic acid (11-Aun).

38. The method of claim 37, wherein Y is 8-aminooctanoic acid.

39. The complex of claim 35, wherein Y is gly-ser-gly.

40. The method of claim 32, wherein X is selected from the group consisting of DOTA, DTPA, S₄, N₃S, N₂S₂, NS₃ and derivatives thereof.

41. The method of claim 40, wherein X is DOTA or a derivative thereof.

42. The method of claim 40, wherein X is N₃S or a derivative thereof.

43. A method of preparing a therapeutic or diagnostic compound that binds a gastrin releasing peptide receptor on a cell surface and is internalized within the cell, said method comprising reacting a metal with a compound having a structure of the formula X—Y—B wherein X is a metal chelating group, Y is a spacer group or covalent bond and B is a gastrin releasing peptide receptor agonist, thereby forming said therapeutic or diagnostic compound.

44. The method of claim 43, wherein the metal is selected from the group consisting of transition metals, lanthanides, auger-electron emitting isotopes, and α-, β- or γ-emitting isotopes.

45. The method of claim 44, wherein the metal is selected from the group consisting of: ¹⁰⁵Rh—, ^{99m}Tc—, ^{186/188}Re—, ¹⁵³Sm—, ¹⁶⁶Ho—, ¹¹¹In—, ⁹⁰Y—, ¹⁷⁷Lu—, ¹⁴⁹Pm—, ⁶⁶Dy—, ¹⁷⁵Yb—, ¹⁹⁹Au— and ^{117m}Sn—.

46. The method of claim 43, wherein Y is selected is selected from the group consisting of at least one amino acid residue, a hydrocarbon chain and a combination thereof.

47. The method of claim 46, wherein Y is a combination of L-glutamine and a hydrocarbon chain.

48. The method of claim 46, wherein Y is selected from the group consisting of glycine, β-alanine, gamma-aminobutanoic acid, 5-aminovaleric acid (5-Ava), 6-aminohexanoic acid, 7-aminoheptanoic acid, 8-aminooctanoic acid (8-Aoc), 9-aminononanoic acid, 10-aminodecanoic acid and 11-aminoundecanoic acid (11-Aun).

49. The method of claim 48, wherein Y is 8-aminooctanoic acid.

50. The method of claim 46, wherein Y is Gly-Ser-Gly.

51. The method of claim 43, wherein X is selected from the group consisting of DOTA, DTPA, S4, N₃S, N₂S₂, NS₃ and derivatives thereof.

52. The method of claim 51, wherein X is DOTA or a derivative thereof.

53. The method of claim 51, wherein X is N₃S or a derivative thereof.

54. A method of performing radioimmuno guided surgery (RIGS) on a patient in need thereof, comprising administering to said patient a complex, said complex comprising a metal and a compound having a structure of the formula X—Y—B, wherein X is a metal chelating group, Y is a spacer group or covalent bond and B is a gastrin releasing peptide (GRP) receptor agonist, and the metal is selected from the group consisting of transition metals, lanthanides, auger-electron emitting isotopes, and α-, β- or γ-emitting isotopes,
wherein said complex binds a gastrin releasing peptide receptor on a cell surface, said complex is internalized within the cell and said cell bound to or containing said complex is detected during said surgery.

55. The method of claim 54, further including removing the detected cell during the surgery.

56. The method of claim 54, wherein the cell is a neoplastic cell.

57. The method of claim 54, wherein the cell bound to or containing the complex is detected by hand held radiation instrumentation.

58. The method of claim 54, wherein the metal is selected from the group consisting of transition metals, lanthanides, auger-electron emitting isotopes, and α-, β- or γ-emitting isotopes.

59. The method of claim 58, wherein the metal is selected from the group consisting of: ¹⁰⁵Rh—, ^{99m}Tc—, ^{186/188}Re—, ¹⁵³Sm—, ¹⁶⁶Ho—, ¹¹¹In—, ⁹⁰Y—, ¹⁷⁷Lu—, ¹⁴⁹Pm—, ¹⁶⁶Dy—, ¹⁷⁵Yb—, ¹⁹⁹Au— and ^{117m}Sn—.

60. The method of claim 54, wherein Y is selected from the group consisting of at least one amino acid residue, a hydrocarbon chain and a combination thereof.

61. The method of claim 60, wherein Y is a combination of L-glutamine and a hydrocarbon chain.

62. The method of claim 60, wherein Y is selected from the group consisting of glycine, β-alanine, gamma-aminobutanoic acid, 5-aminovaleric acid (5-Ava), 6-aminohexanoic acid, 7-aminoheptanoic acid, 8-aminooctanoic acid (8-Aoc), 9-aminononanoic acid, 10-aminodecanoic acid and 11-aminoundecanoic acid (11-Aun).

63. The method of claim 62, wherein Y is 8-aminooctanoic acid.

64. The method of claim 60, wherein Y is Gly-Ser-Gly.

65. The method of claim 54, wherein X is selected from the group consisting of DOTA, DTPA, S₄, N₃S, N₂S₂, NS₃ and derivatives thereof.

66. The method of claim 65, wherein X is DOTA or a derivative thereof.

67. The method of claim 65, wherein X is N₃S or a derivative thereof.
